(19) United States
(12) Patent Application Publication
(10) Pub. No.: US 2005/0149002 A1
(43) Pub. Date: Jul. 7, 2005

(54) Markers for visualizing interventional medical devices

(76) Inventors: Xingwu Wang, Wellsville, NY (US); Frank G. Shellock, Los Angeles, CA (US)

Correspondence Address: HOWARD J. GREENWALD P.C., 349 W. COMMERCIAL STREET SUITE 2490, EAST ROCHESTER, NY, 14445-2408, US

(21) Appl. No.: 10/999,185

(22) Filed: Nov. 29, 2004

Related U.S. Application Data
(63) Continuation-in-part of application No. 10/974,412, filed on Oct. 27, 2004.
Application No. 10/974,412 is a continuation-in-part of application No. 10/950,148, filed on Sep. 24, 2004.
Application No. 10/950,148 is a continuation-in-part of application No. 10/923,579, filed on Aug. 20, 2004.
Application No. 10/923,579 is a continuation-in-part of application No. 10/914,691, filed on Aug. 9, 2004.
Application No. 10/923,579 is a continuation-in-part of application No. 10/887,521, filed on Jul. 7, 2004.
Application No. 10/923,579 is a continuation-in-part of application No. 10/867,517, filed on Jun. 14, 2004.
Application No. 10/923,579 is a continuation-in-part of application No. 10/810,916, filed on Mar. 26, 2004, now Pat. No. 6,846,985.
Application No. 10/923,579 is a continuation-in-part of application No. 10/808,618, filed on Mar. 24, 2004.
Application No. 10/923,579 is a continuation-in-part of application No. 10/786,198, filed on Feb. 25, 2004.
Application No. 10/923,579 is a continuation-in-part of application No. 10/780,045, filed on Feb. 17, 2004.
Application No. 10/923,579 is a continuation-in-part of application No. 10/747,472, filed on Dec. 29, 2003.
Application No. 10/923,579 is a continuation-in-part of application No. 10/744,543, filed on Dec. 22, 2003.
Application No. 10/923,579 is a continuation-in-part of application No. 10/442,420, filed on May 21, 2003.
Application No. 10/923,579 is a continuation-in-part of application No. 10/409,505, filed on Apr. 8, 2003, now Pat. No. 6,815,609.

(51) Int. Cl. A61B 17/00

(52) U.S. Cl. 606/1

(57) ABSTRACT

A marking material that, when disposed upon medical devices used during interventional medical procedures with imaging modalities such as X-ray Fluoroscopy and Magnetic Resonance Imaging, renders such medical devices visible with minimal imaging artifacts. The material comprises a particulate material with generally higher atomic weight disposed within a matrix material with generally lower atomic weight. In one embodiment the particulate material is magnetic. In another embodiment the particulate material is non-magnetic.

### CROSS-REFERENCE TO RELATED PATENT APPLICATIONS

This application is a continuation-in-part of applicants' U.S. patent application Ser. No. 10/974,412, file on Oct. 27, 2004, which in turn was a continuation-in-part of applicants' U.S. patent application Ser. No. 10/950,148, filed on Sep. 24, 2004, which in turn was a continuation-in-part of applicants' patent application Ser. No. 10/923,579, filed on Aug. 20, 2004, which in turn was a continuation-in-part of each of applicants' copending patent application Ser. No. 10/914,691 (filed on Aug. 8, 2004), Ser. No. 10/887,521 (filed on Jul. 7, 2004), Ser. No. 10,867,517 (filed on Jun. 14, 2004), Ser. No. 10/810,916 (filed on Mar. 26, 2004), Ser. No. 10/808,618 (filed on Mar. 24, 2004), Ser. No. 10/786,198 (filed on Feb. 25, 2004), Ser. No. 10/780,045 (filed on Feb. 17, 2004), Ser. No. 10/747,472 (filed on Dec. 29, 2003), Ser. No. 10/744,543 (fled on Dec. 22, 2003), Ser. No. 10/442,420 (filed on May 21, 2003), and Ser. No. 10/409,505 (flied on Apr. 8, 2003). The entire disclosure of each of these patent applications is hereby incorporated by reference into this specification.

### FIELD OF THE INVENTION

This invention relates in general to markers for rendering visible devices used in interventional medical procedures and, more particularly to such markers for devices use in procedures carried out using the imaging modalities of X-ray based fluoroscopy and Magnetic Resonance Imaging.

### BACKGROUND OF THE INVENTION

U.S. Pat. No. 5,782,764, the entire disclosure of which is hereby incorporated by reference into this specification, discusses interventional medical procedures and states: “Medical therapy performed on a patient while the patient is being imaged using a diagnostic imaging modality is generally referred to as interventional radiology or interventional imaging. In interventional radiological procedures, the patient and at least one instrument necessary to perform a therapeutic or diagnostic function, are positioned in the imaging region of an imaging system. Examples of instruments used in interventional radiology are scalpels, forceps, retractors, biopsy needles, catheters, and the like. Implanted devices used for therapy such as sutures, pacemakers, stents, shunts, orthopedic devices, dental devices and the like are also examples of such instruments. It is necessary that the presence of these devices does not lead to a degradation or distortion in the images obtained which would lead to a loss in diagnostic yield. Further, it is often desired that the interventional imaging system portray one or more of the instruments while also imaging a selected portion of the patient. For example, it may be desirable to visualize a biopsy needle or catheter inserted into the tissue of the patient. In addition, it is also desirable to have permanently implanted medical devices such as blood filters, stents, or other such implants which do not distort or degrade the images obtained with the imaging system. Diagnostic yield may be maximized by the use of implanted devices which may be visualized in the image, as a structure distinct from the surrounding tissue, without affecting the image quality of the surrounding tissue structures.”

X-ray fluoroscopy is the most commonly used imaging modality for interventional procedures. U.S. Pat. No. 6,574,497, the entire disclosure of which is hereby incorporated by reference into this specification, discusses X-ray fluoroscopy and its advantages and disadvantages, stating that “Currently, x-ray fluoroscopy is the preferred imaging modality for cardiovascular interventional procedures. No other method, at this time, has the temporal or spatial resolution of fluoroscopy. As good as fluoroscopy is, however, it does have drawbacks. Catheterization is required in order to directly inject the high concentration of iodinated contrast agent required. Systemic administration of the contrast agent would require too high a dose of agent. Additionally, iodinated contrast agents are nephrotoxic with a real incidence of acute renal failure, particularly in patients with compromised renal function. Allergic reactivity also serves as a contraindication for certain patients. Visualization and tracking of devices under fluoroscopy is accomplished either by the device's inherent adsorption of x-rays, or by the placement of radiopaque markers. Fluoroscopy generates a compressed, two dimensional image of what are three dimensional structures. This requires multiple views to appraise complex vasculature. Moreover, fluoroscopy uses ionizing x-ray radiation with its attendant hazards. This is an issue for the patient during protracted or repeated interventions. It is a daily issue for the interventionalist who must also cope with the burden of personal dose monitoring and wearing lead shielding.”

U.S. Pat. No. 6,574,497 further states: “One imaging modality, which has the potential to supplant fluoroscopy, or perhaps replace it in the long term, is magnetic resonance imaging (MRI). MRI does not use ionizing radiation and does not require catheterization to image vasculature. MRI contrast agents, which are often necessary for best resolution, are much less nephrotoxic than iodinated fluoroscopy agents and are effective when administered intravenously.”

U.S. Pat. No. 6,574,497 further states: “As noted above, MRI angiography is an active area of research. Device tracking and visualization under MRI is necessary for MRI guided interventions. Plastic devices show up poorly under MRI. The reason is that even though the majority of polymers contain hydrogen nuclei, the resonance signals from protons in polymers are broad and chemically shifted from protons in water from which the majority of the MRI signal is derived. Polymeric catheters, for example, show up as regions of little or no signal under MRI (signal voids). Hence, there is a need for markers to track and visualize interventional devices.

MRI markers are divided into two categories, active and passive. Active markers, as the name implies, participate in the radio frequency signal transmission or reception of the scanner. This includes markers that emit an RF signal, markers that receive an RF signal and convey it to the scanner via a connection, and markers that generate their own magnetic or electrical field by application of electrical currents. The term active implies some sort of electrical circuit is involved. Conversely, passive markers use no wires or circuitry and work by several mechanisms. One scheme is to distort the magnetic field of the scanner. Another is by enhancing or modifying the signal from protons in the vicinity. Still another is by containing nuclei with their own distinct signal that is different from water or fat. Passive markers have the advantage that they are simpler and, generally, have fewer parts. They require no connection to the scanner or additional circuitry. There also may be the perception amongst physicians that active currents and voltages in or on interventional devices create additional safety issues to be managed. Lastly, passive markers are conceptually similar to the radiopaque markers in fluoroscopy, even if they work in a very different way.

There are two main types of passive markers being proposed. One is based on magnetic susceptibility. This usually includes paramagnetic or ferromagnetic particles, bands, or other components placed in or on the device. These materials perturb the magnetic field in the vicinity of the device. This alters the resonance condition of protons in the vicinity. The net result is a signal void that appears black in MRI images.

The second scheme uses the currently approved gadolinium contrast agents; however, the contrast agents are placed inside the device. For example, gadolinium contrast solution is used to fill the lumen of a catheter or inflate an angioplasty balloon. In T.sub.1 weighted images, aqueous solutions of gadolinium show a signal enhancement due to the T.sub.1 shortening effect of the gadolinium. Gadolinium also shortens T.sub.2 and gives some enhancement in those images as well. In contrast to the susceptibility artifact which is dark, an aqueous gadolinium solution marker shows up bright.

Another mechanism is possible if the medical device contains nuclei other than protons. In this case, it is possible to track the device due to the distinctive signal of this other nuclei, especially its frequency. Protons, hydrogen nuclei, have the advantage that they are abundant and have very good MRI sensitivity. They also have only two allowed spin states (nuclear spin=½). Nuclei with a spin greater than ½ have a quadrapole dipole moment, which broadens their NMR resonance signal. Fluorine-19 has reasonable sensitivity compared to .sup.1 H and a resonant frequency that can be accommodated by the RF equipment in current scanners. Fluorine-19 also has a spin quantum number of ½, like hydrogen nuclei, giving it a sharp NMR signal.

What has been needed, and heretofore unavailable, in the art of interventional magnetic resonance angiography are medical devices (such as guidewires, catheters and implantable prostheses, e.g., stents) which contain passive markers for visualization under MRI. Such medical devices should provide a visible indication of the device during MRI angiography, without reliance upon susceptibility artifacts and signal voids.”

In light of the above, it is the object of this invention to provide markers for interventional medical devices that may be used with X-ray fluoroscopy and Magnetic Resonance Imaging and produce minimal image artifacts in either of those imaging modalities.

### SUMMARY OF THE INVENTION

In accordance with one embodiment of this invention, there is provided a material that, when disposed upon medical devices used during interventional medical procedures with imaging modalities such as X-ray Fluoroscopy and Magnetic Resonance Imaging, renders such medical devices visible with minimal imaging artifacts. The material comprises a particulate material with generally higher atomic weight disposed within a matrix material with generally lower atomic weight. In one embodiment the particulate material is magnetic. In another embodiment the particulate material is non-magnetic. Detailed descriptions of the particulate material and the matrix material in accordance with various embodiments of the invention will be disclosed below.

### BREIF DESCRIPTION OF THE DRAWINGS

Applicants' inventions will be described by reference to the specification and the drawings, in which like numerals refer to like elements, and wherein:

FIG. 1 is a schematic illustration, not drawn to scale, of a coated substrate assembly 10 comprised of a substrate 12 and, disposed thereon, a coating 14 comprised of a multiplicity of nanomagnetic particles 16;

FIGS. 2 and 3 schematically illustrate the porosity of the side of coating 14, and the top of the coating 14, depicted in FIG. 1;

FIG. 4 is a schematic illustration of a coated stent assembly 100;

FIG. 4A is a schematic sectional view of a coated substrate comprised of a via;

FIG. 4B is a schematic of an arrangement of coating layers that create capacitance in parallel;

FIG. 4C is a schematic of an arrangement of coating layers that creates capacitance in series;

FIG. 4D is a schematic of an arrangement of coating layers that creates inductance in series;

FIG. 4E is a schematic of an arrangement of coating layers that creates inductance in parallel;

FIG. 5 is a partial schematic view of a coated stent assembly 200;

FIG. 6 is a schematic of one preferred sputtering process;

FIG. 7 is a partial schematic of one preferred particle collection process;

FIG. 8 is a schematic of a plasma deposition process;

FIG. 9 is a schematic of one preferred forming process;

FIGS. 10, 11, 12, 13, and 14 are schematic illustrations of preferred particles of the invention;

FIG. 15 is a phase diagram showing various compositions that may contain moieties E, F, and G;

FIG. 16 is a cross-sectional view of a preferred stent of this invention;

FIG. 17 is a cross-sectional view of a coated strut 1020 of the stent of FIG. 16;

FIG. 18 shows the effect on the coated strut 1020 when a patient is exposed to an electromagnetic field 1090;

FIG. 19 is a cross-sectional view of another coated strut 1021;

FIG. 20 shows the effect on the coated strut 1021 when a patient is exposed to an electromagnetic field 1090;

FIG. 21 is a cross-sectional view of another coated strut 1023;

FIG. 22 shows the effect on the coated strut 1023 when a patient is exposed to an electromagnetic field 1090;

FIG. 23 is a cross-sectional view of a coated strut 1027;

FIG. 24 is a schematic of one preferred stent assembly of this invention;

FIG. 25 is a graph of the input electromagnetic wave, and the output electromagnetic wave, depicted in the stent assembly of FIG. 24;

FIG. 26 is a sectional view of strut of one preferred stent of the invention;

FIG. 27 is a schematic sectional view of one preferred coated substrate;

FIG. is 28 is an equivalent circuit representing the electrical phenomena that occur when the substrate of FIG. 27 is subjected to an MRI field;

FIG. 29 is a schematic illustration of the various sections of a nanomagnetic coating and how its dielectric properties vary from section to section;

FIG. 30 is a B/H graph of a particular nanomagnetic coating;

FIG. 31 is a schematic of an apparatus for testing the magnetic properties of a sample;

FIG. 32 is a schematic illustration of a coated substrate wherein one or more of the coatings on the substrate are discontinuous and are separated by one or more vias;

FIG. 33 is a schematic of a device for testing the degree to which the Faraday Cage effect blocks the transmission of radio-frequency energy in a coated stent;

FIG. 34 is a schematic illustration of a material according to one embodiment of the invention.

### DETAILED DESCRIPTION OF THE INVENTION

Nanomagnetic Embodiment of the Invention

In one embodiment of this invention, the marker material is comprised of a nanomagnetic material such as has been described by applicants in several of their prior United States patents. Reference may be had, e.g., to U.S. Pat. No. 6,506,972 (magnetically shielded conductor), U.S. Pat. No. 6,673,999 (magnetically shielded assembly), U.S. Pat. No. 6,700,472 (magnetic thin film inductors), U.S. Pat. No. 6,713,671 (magnetically shielded assembly), and U.S. Pat. No. 6,765,144 (magnetic resonance imaging coated assembly). The entire disclosure of each of these United States patents, especially as it relates to nanomagnetic material, is hereby incorporated by reference into this specification.

In one embodiment the magnetic permeability of the particulate material is greater than the magnetic permeability of the matrix material, being at least 1.000005 to 20,000 times as great. As used in this specification, the term “magnetic permeability” refers to “ . . . a property of materials modifying the action of magnetic poles placed therein and modifying the magnetic induction resulting when the material is subjected to a magnetic field of magnetizing force. The permeability of a substance may be defined as the ratio of the magnetic induction in the substance to the magnetizing field to which it is subjected. The permeability of a vacuum is unity.” See, e.g., page F-102 of—Robert E. Weast et al.'s “Handbook of Chemistry and Physics,” 63^{rd} Edition (CRC Press, Inc., Boca Raton, Fla., 1982-1983 edition). Reference may also be had, e.g., to U.S. Pat. No. 4,007,066 (material having a high magnetic permeability), U.S. Pat. No. 4,340,770 (enhancement of the magnetic permeability in glass metal shielding), U.S. Pat. No. 4,482,397 (method for improving the magnetic permeability of grain oriented silicon steel), U.S. Pat. No. 4,702,935 (high magnetic permeability alloy film), U.S. Pat. No. 4,725,490 (high magnetic permeability composites containing fibers with ferrite fill) U.S. Pat. No. 5,073,211 (method for manufacturing steel article having high magnetic permeability and low coercive force), U.S. Pat. No. 5,099,518 (electrical conductor of high magnetic permeability material), U.S. Pat. No. 5,645,774 (method for establishing a target magnetic permeability in a ferrite), U.S. Pat. No. 5,691,645 (process for determining intrinsic magnetic permeability of elongated ferromagnetic elements), U.S. Pat. No. 5,691,645 (process for determining intrinsic magnetic permeability of elongated ferromagnetic elements), U.S. Pat. No. 6,020,741 (wellbore imaging using magnetic permeability measurements), U.S. Pat. No. 6,176,944 (method for making low magnetic permeability cobalt sputter targets), U.S. Pat. No. 6,190,516 (high magnetic flux sputter targets with varied magnetic permeability in selected regions), U.S. Pat. No. 6,233,126 (thin film magnetic head having low magnetic permeability layer), U.S. Pat. No. 6,472,836 (magnetic permeability position detector), and the like. The entire disclosure of each of these United States patents is hereby incorporated by reference into this specification. Reference may also be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel.

Nanomagnetic Particles in the Nanomagnetic Material of this Embodiment

In this embodiment of the invention, the particulate material comprises nanomagnetic particles that may be in the form of a film, a powder, a solution, etc.

The nanomagnetic material of this embodiment of the invention is generally comprised of at least about 0.05 weight percent of such nanomagnetic particles and, preferably, at least about 5 weight percent of such nanomagnetic particles. In one embodiment, such nanomagnetic material is comprised of at least about 50 weight percent of such magnetic particles. In another embodiment, such nanomagnetic material consists essentially of such nanomagnetic particles.

When the collection of nanomagnetic particles consists essentially of nanomagnetic particles, the term “compact” may be used to refer to such collection of nanomagnetic particles.

Particle Size of the Nanomagnetic Particles of this Embodiment

In general, the nanomagnetic particles of this invention are smaller than about 100 nanometers. In one embodiment, these nano-sized particles have a particle size distribution such that at least about 90 weight percent of the particles have a maximum dimension in the range of from about 1 to about 100 nanometers.

In one embodiment, the average size of the nanomagnetic particles is preferably less than about 50 nanometers. In one embodiment, the nanomagnetic particles have an average size of less than about 20 nanometers. In another embodiment, the nanomagnetic particles have an average size of less than about 15 nanometers. In yet another embodiment, such average size is less than about 11 nanometers; in one aspect of this embodiment, such average size is from about 3 to about 10 nanometers. In yet another embodiment, such average size is less than about 3 nanometers.

Coherence Length of the Nanomagnetic Particles of this Embodiment

As is used in this specification, the term “coherence length” refers to the distance between adjacent nanomagnetic moieties, and it has the meaning set forth in applicants' published international patent document W003061755A2, the entire disclosure of which is hereby incorporated by reference into this specification. As is disclosed in such published international patent document, “Referring to FIG. 38, and in the preferred embodiment depicted therein, it will be seen that A moieties 5002, 5004, and 5006 are separated from each other either at the atomic level and/or at the nanometer level. The A moieties may be, e.g., A atoms, clusters of A atoms, A compounds, A solid solutions, etc; regardless of the form of the A moiety, it has the magnetic properties described hereinabove . . . Thus, referring . . . to FIG. 38, the normalized magnetic interaction between adjacent A moieties 5002 and 5004, and also between 5004 and 5006, is preferably described by the formula M=exp (−x/L), wherein M is the normalized magnetic interaction, exp is the base of the natural logarithm (and is approximately equal to 2.71828), x is the distance between adjacent A moieties, and L is the coherence length . . . . In one embodiment, and referring again to FIG. 38, x is preferably measured from the center 5001 of A moiety 5002 to the center 5002 of A moiety 5004; and x is preferably equal to from about 0.00001×L to about 100×L . . . . In one embodiment, the ratio of x/L is at least 0.5 and, preferably, at least 1.”

With regard to the term “coherence length,” reference also may be had to U.S. Pat. No. 4,411,959 (which discloses that “ . . . the spherical particle diameter, phi, preferably is to exceed the Ginzburg-Landau coherence lengths, .xi.GL, to avoid any significant degradation of Tc. The spacing between adjacent particles is to be much less than .xi.GL to ensure strong coupling while the diameter of voids between dense-packed spheres should be comparable to .xi.GL in order to ensure maximum flux pinning . . . ”), U.S. Pat. No. 5,098,178 (which discloses that “In addition, the anisotropic shrinkage of the Sol-Gel during polymerization is utilized to increase the concentration of the superconducting inclusions 22 so that the average particle distance . . . between the superconducting inclusions 22 approaches the coherence length as much as possible. An average particle distance comparable to the coherence length between the superconducting inclusions 22 is necessary in order to achieve significant enhancement through the proximity effect and high critical currents for the matrix 10.”), U.S. Pat. No. 5,998,336 (“The ceramic particles 2 have physical dimensions larger than the superconducting coherence length of the ceramic. Typically, the coherence length of high T_{c} ceramic materials is 1.5 nm.”), U.S. Pat. No. 6,420,318 (“The particles 22 preferably have dimensions larger than the superconducting coherence length of the superconducting material.”), and the like. The entire disclosure of each of these United States patents is hereby incorporated by reference into this specification. The coherence length (L) between adjacent magnetic particles is, on average, preferably from about 10 to about 200 nanometers and, more preferably, from about 50 to about 150 nanometers. In one preferred embodiment, the coherence length (L) between adjacent nanomagnetic particles is from about 75 to about 125 nanometers.

In one embodiment, x is preferably equal to from about 0.00001 times L to about 100 times L. In one embodiment, the ratio of x/L is at least 0.5 and, preferably, at least 1.5.

Ratio of the Coherence Length Between Nanomagnetic Particles to their Particle Size

In one preferred embodiment, the ratio of the coherence length between adjacent nanomagnetic particles to their particle size is at least 2 and, preferably, at least 3. In one aspect of this embodiment, such ratio is at least 4. In another aspect of this embodiment, such ratio is at least 5.

The Saturation Magnetization of the Nanomagnetic Particles of this Embodiment

The nanomagnetic particles of this invention preferably have a saturation magnetization (“magnetic moment”) of from about 2 to about 3,000 electromagnetic units (emu) per cubic centimeter of material. As is known to those skilled in the art, saturation magnetization is the maximum possible magnetization of a material. Reference may be had, e.g., to U.S. Pat. No. 3,901,741 (saturation magnetization of cobalt, samarium, and gadolinium alloys), U.S. Pat. No. 4,134,779 (iron-boron solid solution alloys having high saturation magnetization), U.S. Pat. No. 4,390,853 (microwave transmission devices having high saturation magnetization and low magnetostriction), U.S. Pat. No. 4,532,979 (iron-boron solid solution alloys having high saturation magnetization and low magnetostriction), U.S. Pat. No. 4,631,613 (thin film head having improved saturation magnetization), U.S. Pat. No. 4,705,613, 4,782,416 (magnetic head having two legs of predetermined saturation magnetization for a recording medium to be magnetized vertically), U.S. Pat. No. 4,894,360 (method of using a ferromagnetic material having a high permeability and saturation magnetization at low temperatures), U.S. Pat. No. 5,543,070 (magnetic recording powder having low curie temperature and high saturation magnetization), U.S. Pat. No. 5,761,011 (magnetic head having a magnetic shield film with a lower saturation magnetization than a magnetic response film of an MR element), U.S. Pat. No. 5,922,442 (magnetic recording medium having a cobalt/chromium alloy interlayer of a low saturation magnetization), U.S. Pat. No. 6,492,035 (magneto-optical recording medium with intermediate layer having a controlled saturation magnetization), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

Saturation magnetization may be measured by conventional means. Reference may be had, e.g., to U.S. Pat. No. 5,068,519 (magnetic document validator employing remanence and saturation measurements), U.S. Pat. Nos. 5,581,251, 6,666,930, 6,506,264 (ferromagnetic powder), U.S. Pat. Nos. 4,631,202, 4,610,911, 5,532,095, and the like. The entire disclosure of each of these United States patents is hereby incorporated by reference into this specification.

In one embodiment, the saturation magnetization of the nanomagnetic particles of this invention is preferably measured by a SQUID (superconducting quantum interference device). Reference may be had, e.g., to U.S. Pat. No. 5,423,223 (fatigue detection in steel using squid mangetometry), U.S. Pat. No. 6,496,713 (ferromagnetic foreign body detection with background canceling), U.S. Pat. Nos. 6,418,335, 6,208,884 (noninvasive room temperature instrument to measure magnetic susceptibility variations in body tissue), U.S. Pat. No. 5,842,986 (ferromagnetic foreign body screening method), U.S. Pat. Nos. 5,471,139, 5,408,178, and the like. The entire disclosure of each of these United States patents is hereby incorporated by reference into this specification.

In one preferred embodiment, the saturation magnetization of the nanomagnetic particle of this invention is at least 100 electromagnetic units (emu) per cubic centimeter and, more preferably, at least about 200 electromagnetic units (emu) per cubic centimeter. In one aspect of this embodiment, the saturation magnetization of such nanomagnetic particles is at least about 1,000 electromagnetic units per cubic centimeter.

In another embodiment, the nanomagnetic material of this invention is present in the form a film with a saturation magnetization of at least about 2,000 electromagnetic units per cubic centimeter and, more preferably, at least about 2,500 electromagnetic units per cubic centimeter. In this embodiment, the nanomagnetic material in the film preferably has the formula A₁A₂(B)ₓC₁ (C₂)_{y}, wherein y is 1, the C moieties are oxygen and nitrogen, respectively, and the A moieties and the B moiety are as described elsewhere in this specification.

In one embodiment, the saturation magnetization of the nanomagnetic material is greater than about 1.5 Tesla. In another embodiment, the saturation magnetization of the nanomagnetic material is greater than about 3.0 Tesla.

Without wishing to be bound to any particular theory, applicants believe that the saturation magnetization of their nanomagnetic particles may be varied by varying the concentration of the “magnetic” moiety A in such particles, and/or the concentrations of moieties B and/or C.

In one embodiment, in order to achieve the desired degree of saturation magnetization, the nanomagnetic particles used typically comprise one or more of iron, cobalt, nickel, gadolinium, and samarium atoms. Thus, e.g., typical nanomagnetic materials include alloys of iron and nickel (permalloy), cobalt, niobium, and zirconium (CNZ), iron, boron, and nitrogen, cobalt, iron, boron, and silica, iron, cobalt, boron, and fluoride, and the like. These and other materials are described in a book by J. Douglas Adam et al. entitled “Handbook of Thin Film Devices” (Academic Press, San Diego, Calif., 2000). Chapter 5 of this book, beginning at page 185, describes “magnetic films for planar inductive components and devices;” and Tables 5.1 and 5.2 in this chapter describe many magnetic materials.

The Coercive Force of the Nanomagnetic Particles of this Embodiment

In one preferred embodiment, the nanomagnetic particles of this invention have a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 3,982,276, 4,003,813 (method of making a magnetic oxide film with a high coercive force), U.S. Pat. No. 4,045,738 (variable reluctance speed sensor using a shielded high coercive force rare earth magnet), U.S. Pat. Nos. 4,061,824, 4,115,159 (method of increasing the coercive force of pulverized rare earth-cobalt alloys) U.S. Pat. No. 4,277,552 (toner containing high coercive force magnetic powder), U.S. Pat. No. 4,396,441 (permanent magnet having ultra-high coercive force), U.S. Pat. No. 4,465,526 (high coercive force permanent magnet), U.S. Pat. No. 4,481,045 (high-coercive-force permanent magnet), U.S. Pat. No. 4,485,163 (triiron tetroxide having specified coercive force), U.S. Pat. No. 4,675,170 (preparation of finely divided acicular hexagonal ferrites having a high coercive force), U.S. Pat. Nos. 4,741,953, 4,816,933 (magnetic recording medium of particular coercive force), U.S. Pat. No. 4,863,530 (Fc—Pt—Nb magnet with ultra-high coercive force), U.S. Pat. Nos. 4,939,210, 5,073,211 (method for manufacturing steel article having high magnetic permeability and low coercive force), U.S. Pat. No. 5,211,770 (magnetic recording powder having a high coercive force at room temperatures and a low curie point), U.S. Pat. No. 5,329,413 (magnetoresistive sensor magnetically coupled with high-coercive force film at two end regions), U.S. Pat. No. 5,596,555 (magnetooptical recording medium having magnetic layers that satisfy predetermined coercive force relationships), U.S. Pat. No. 5,686,137 (method of providing hexagonal ferrite magnetic powder with enhanced coercive force stability), U.S. Pat. No. 5,742,458 (giant magnetoresistive material film which includes a free layer, a pinned layer, and a coercive force increasing layer), U.S. Pat. Nos. 5,967,223, 6,189,791 (magnetic card reader and method for determining the coercive force of a magnetic card therein), U.S. Pat. Nos. 6,257,512, 6,295,186, 6,637,653 (method of measuring coercive force of a magnetic card), U.S. Pat. No. 6,449,122 (thin-film magnetic head including soft magnetic film exhibiting high saturation magnetic flux density and low coercive force), U.S. Pat. No. 6,496,338 (spin-valve magnetoresistive sensor including a first antiferromagnetic layer for increasing a coercive force), U.S. Pat. No. 6,667,119 (magnetic recording medium comprising magnetic layers, the coercive force thereof specifically related to saturation magnetic flux density), U.S. Pat. No. 6,687,009 (magnetic head with conductors formed on endlayers of a multilayer film having magnetic layer coercive force difference), and the like. The entire disclosure of each of these United States patents is hereby incorporated by reference into this specification.

In one embodiment, the nanomagnetic particles have a coercive force of from about 0.01 to about 3,000 Oersteds. In yet another embodiment, the nanomagnetic particles have a coercive force of from about 0.1 to about 10.

The Phase Transition Temperature of the Nanomagnetic Particles of this Embodiment

In one embodiment of this invention, the nanomagnetic particles have a phase transition temperature is from about 40 degrees Celsius to about 200 degrees Celsius. As used herein, the term phase transition temperature refers to temperature in which the magnetic order of a magnetic particle transitions from one magnetic order to another. Thus, for example, when a magnetic particle transitions from the ferromagnetic order to the paramagnetic order, the phase transition temperature is the Curie temperature. Thus, e.g., when the magnetic particle transitions from the anti-ferromagnetic order to the paramagnetic order, the phase transition temperature is known as the Neel temperature.

For a discussion of phase transition temperature, reference may be had, e.g., to U.S. Pat. No. 4,804,274 (method and apparatus for determining phase transition temperature using laser attenuation), U.S. Pat. No. 5,758,968 (optically based method and apparatus for detecting a phase transition temperature of a material of interest), U.S. Pat. Nos. 5,844,643, 5,933,565 (optically based method and apparatus for detecting a phase transition temperature of a material of interest), U.S. Pat. No. 6,517,235 (using refractory metal silicidation phase transition temperature points to control and/or calibrate RTP low temperature operation), and the like. The entire disclosure of each of these United States patents is hereby incorporated by reference into this specification.

For a discussion of Curie temperature, reference may be had, e.g., to U.S. Pat. No. 3,736,500 (liquid identification using magnetic particles having a preselected Curie temperature), U.S. Pat. No. 4,229,234 (passivated, particulate high Curie temperature magnetic alloys), U.S. Pat. Nos. 4,771,238, 4,778,867 (ferroelectric copolymers of vinylidene fluoride and trifluoroethyelene), U.S. Pat. No. 5,108,191 (method and apparatus for determining Curie temperatures of ferromagnetic materials), U.S. Pat. No. 5,229,219 (magnetic recording medium having a Curie temperature up to 180 degrees C.), U.S. Pat. No. 5,325,343 (magneto-optical recording medium having two RE-TM layers with the same Curie temperature), U.S. Pat. No. 5,420,728 (recording medium with several recording layers having different Curie temperatures), U.S. Pat. No. 5,487,046 (magneto-optical recording medium having two magnetic layers with the same Curie temperature), U.S. Pat. No. 5,543,070 (magnetic recording powder having low Curie temperature and high saturation magnetization), U.S. Pat. Nos. 5,563,852, 601,742 (heating device for an internal combustion engine with PTC elements having different Curie temperatures), U.S. Pat. No. 5,679,474 (overwritable optomagnetic recording medium having a layer with a Curie temperature that varies in the thickness direction), U.S. Pat. No. 5,764,601 (magneto-optical recording medium with a readout layer of varying composition and Curie temperature), U.S. Pat. Nos. 5,949,743, 6,125,083 (magneto-optical recording medium containing a middle layer with a lower Curie temperature than the other layers), U.S. Pat. No. 6,731,111 (magnetic ink containing magnetic powders with different Curie temperatures), and the like. The entire disclosure of each of these United States patents is hereby incorporated by reference into this specification.

As used herein, the term “Curie temperature” refers to the temperature marking the transition between ferromagnetism and paramagnetism, or between the ferroelectric phase and paraelectric phase. This term is also sometimes referred to as the “Curie point.”

As used herein, the term “Neel temperature” refers to a temperature, characteristic of certain metals, alloys, and salts, below which spontaneous magnetic ordering takes place so that they become antiferromagnetic, and above which they are paramagnetic; this is also known as the Neel point. Reference may be had, e.g., to U.S. Pat. Nos. 3,845,306; 3,883,892; 3,946,372; 3,971,843; 4,103,315; 4,396,886; 5,264,980; 5,492,720; 5,756,191; 6,083,632; 6,181,533, 3,883,892, 3,845,306; 6,020,060; 6,083,632, 4,396,886, 4,438,462; 4,621,030; 5,923,504; 6,020,060; 6,146,752; 6,483,674; 6,631,057; 6,534,204; 6,534,205; 6,754,720; and the like. The entire disclosure of each of these United States patents is hereby incorporated by reference into this specification.

Neel temperature is also discussed at page F-92 of the “Handbook of Chemistry and Physics,” 63^{rd} Edition (CRC Press, Inc., Boca Raton, Fla., 1982-1983). As is disclosed on such page, ferromagnetic materials are “those in which the magnetic moments of atoms or ions tend to assume an ordered but nonparallel arrangement in zero applied field, below a characteristic temperature called the Neel point. In the usual case, within a magnetic domain, a substantial net magnetization results form the antiparallel alignment of neighboring nonequivalent sublattices. The macroscopic behavior is similar to that in ferromagnetism. Above the Neel point, these materials become paramagnetic.”

Without wishing to be bound to any particular theory, applicants believe that the phase temperature of their nanomagnetic particles can be varied by varying the ratio of the A, B, and C moieties described hereinabove as well as the particle sizes of the nanoparticles.

In one embodiment of this invention, the phase transition temperature of the nanomagnetic particles is less than about 50 degrees Celsius and, preferably, less than about 46 degrees Celsius. In one aspect of this embodiment, such phase transition temperature is less than about 45 degrees Celsius.

The Squareness of the Nanomagnetic Particles of this Embodiment of the Invention

As is known to those skilled in the art, the squareness of a magnetic material is the ratio of the residual magnetic flux and the saturation magnetic flux density. Reference may be had, e.g., to U.S. Pat. Nos. 6,627,313, 6,517,934, 6,458,452, 6,391,450, 6,350,505, 6,248,437, 6,194,058, 6,042,937, 5,998,048, 5,645,652, and the like. The entire disclosure of such United States patents is hereby incorporated by reference into this specification. Reference may also be had to page 1802 of the McGraw-Hill Dictionary of Scientific and Technical Terms, Fourth Edition (McGraw-Hill Book Company, New York, N.Y., 1989). At such page 1802, the “squareness ratio” is defined as “The magnetic induction at zero magnetizing force divided by the maximum magnetic indication, in a symmetric cyclic magnetization of a material.”

In one embodiment, the squareness of applicants' nanomagnetic particles is from about 0.05 to about 1.0. In one aspect of this embodiment, such squareness is from about 0.1 to about 0.9. In another aspect of this embodiment, the squareness is from about 0.2 to about 0.8. In applications where a large residual magnetic moment is desired, the squareness is preferably at least about 0.8.

The Diverse Atomic Nature of the Nanomagnetic Particles of this Embodiment

In one embodiment, the nanomagnetic particles are depicted by the formula A₁A₂(B)ₓC₁ (C₂)_{y}, wherein each of A₁ and A₂ are separate magnetic A moieties, as described below; B is as defined elsewhere in this specification; x is an integer from 0 to 1; each of C₁ and C₂ is as descried elsewhere in this specification; and y is an integer from 0 to 1.

The composition of these preferred nanomagnetic particles may be depicted by a phase diagram such as, e.g., the phase diagram depicted in FIGS. 37 et seq. of U.S. Pat. No. 6,765,144, the entire disclosure of which is hereby incorporated by reference into this specification. As is disclosed in such United States patent, “Referring to FIG. 37, and in the preferred embodiment depicted therein, a phase diagram 5000 is presented. As is illustrated by this phase diagram 5000, the nanomagnetic material used in the composition of this invention preferably is comprised of one or more of moieties A, B, and C . . . . The moiety A depicted in phase diagram 5000 is comprised of a magnetic element selected from the group consisting of a transition series metal, a rare earth series metal, or actinide metal, a mixture thereof, and/or an alloy thereof . . . . As is known to those skilled in the art, the transition series metals include chromium, manganese, iron, cobalt, nickel. One may use alloys or iron, cobalt and nickel such as, e.g., iron-aluminum, iron-carbon, iron-chromium, iron-cobalt, iron-nickel, iron nitride (Fe3N), iron phosphide, iron-silicon, iron-vanadium, nickel-cobalt, nickel-copper, and the like. One may use alloys of manganese such as, e.g., manganese-aluminum, manganese-bismuth, MnAs, MnSb, MnTe, manganese-copper, manganese-gold, manganese-nickel, manganese-sulfur and related compounds, manganese-antimony, manganese-tin, manganese-zinc, Heusler alloy, and the like. One may use compounds and alloys of the iron group, including oxides of the iron group, halides of the iron group, borides of the transition elements, sulfides of the iron group, platinum and palladium with the iron group, chromium compounds, and the like.”

U.S. Pat. No. 6,765,144 also discloses that: “One may use a rare earth and/or actinide metal such as, e.g., Ce, Pr, Nd, Pm, Sm, Eu, Gd, Tb, Dy, Ho, Er, Tm, Yb, Lu, La, mixtures thereof, and alloys thereof. One may also use one or more of the actinides such as, e.g., Th, Pa, U, Np, Pu, Am, Cm, Bk, Cf. Es, Fm, Md, No, Lr, Ac, and the like . . . . These moieties, compounds thereof, and alloys thereof are well known and are described, e.g., in the aforementioned text of R. S. Tebble et al. entitled “Magnetic Materials . . . . In one preferred embodiment, moiety A is selected from the group consisting of iron, nickel, cobalt, alloys thereof, and mixtures thereof. In this embodiment, the moiety A is magnetic, i.e., it has a relative magnetic permeability of from about 1 to about 500,000 . . . .”

U.S. Pat. No. 6,765,144 also discloses that “The moiety A also preferably has a saturation magnetization of from about 1 to about 36,000 Gauss, and a coercive force of from about 0.01 to about 5,000 Oersteds . . . . The moiety A may be present in the nanomagnetic material either in its elemental form, as an alloy, in a solid solution, or as a compound . . . It is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.).” In another embodiment, from about 5 to about 15 weight percent of the A moiety, preferably in the form of iron, is present in the nanomagnetic material.

U.S. Pat. No. 6,765,144 also discloses that “In addition to moiety A, it is preferred to have moiety B be present in the nanomagnetic material. In this embodiment, moieties A and B are admixed with each other. The mixture may be a physical mixture, it may be a solid solution, it may be comprised of an alloy of the A/B moieties, etc.

In one embodiment, the magnetic material A is dispersed within nonmagnetic material B. This embodiment is depicted schematically in FIG. 38.”

U.S. Pat. No. 6,765,144 also discloses that “Referring to FIG. 38, and in the preferred embodiment depicted therein, it will be seen that A moieties 5002, 5004, and 5006 are separated from each other either at the atomic level and/or at the nanometer level. The A moieties may be, e.g., A atoms, clusters of A atoms, A compounds, A solid solutions, etc; regardless of the form of the A moiety, it has the magnetic properties described hereinabove . . . . In the embodiment depicted in FIG. 38, each A moiety produces an independent magnetic moment. The coherence length (L) between adjacent A moieties is, on average, from about 0.1 to about 100 nanometers and, more preferably, from about 1 to about 50 nanometers . . . the normalized magnetic interaction between adjacent A moieties 5002 and 5004, and also between 5004 and 5006, is preferably described by the formula M=exp(−x/L), wherein M is the normalized magnetic interaction, exp is the base of the natural logarithm (and is approximately equal to 2.71828), x is the distance between adjacent A moieties, and L is the coherence length.”

U.S. Pat. No. 6,765,144 also discloses that “In one embodiment, and referring again to FIG. 38, x is preferably measured from the center 5001 of A moiety 5002 to the center 5003 of A moiety 5004; and x is preferably equal to from about 0.00001×L to about 100×L . . . . In one embodiment, the ratio of x/L is at least 0.5 and, preferably, at least 1.5.”

U.S. Pat. No. 6,765,144 also discloses that “Referring again to FIG. 37, the nanomagnetic material may be comprised of 100 percent of moiety A, provided that such moiety A has the required normalized magnetic interaction (M). Alternatively, the nanomagnetic material may be comprised of both moiety A and moiety B . . . . When moiety B is present in the nanomagnetic material, in whatever form or forms it is present, it is preferred that it be present at a mole ratio (by total moles of A and B) of from about 1 to about 99 percent and, preferably, from about 10 to about 90 percent . . . . The B moiety, in whatever form it is present, is nonmagnetic, i.e., it has a relative magnetic permeability of 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like . . . . In one embodiment, and without wishing to be bound to any particular theory, it is believed that B moiety provides plasticity to the nanomagnetic material that it would not have but for the presence of B . . . .”

U.S. Pat. No. 6,765,144 also discloses that “The use of the B material allows one to produce a coated substrate with a springback angle of less than about 45 degrees. As is known to those skilled in the art all materials have a finite modulus of elasticity; thus, plastic deformations followed by some elastic recovery when the load is removed. In bending, this recovery is called springback. See, e.g., page 462 of S. Kalparjian's “Manufacturing Engineering and Technology,” Third Edition (Addison Wesley Publishing Company, New York, N.Y., 1995) . . . . FIG. 39 illustrates how springback is determined in accordance with this invention. Referring to FIG. 39, a coated substrate 5010 is subjected to a force in the direction of arrow 5012 that bends portion 5014 of the substrate to an angle 5016 of 45 degrees, preferably in a period of less than about 10 seconds. Thereafter, when the force is released, the bent portion 5014 springs back to position 5018. The springback angle 5020 is preferably less than 45 degrees and, preferably, is less than about 10 degrees.”

U.S. Pat. No. 6,765,144 also discloses that “Referring again to FIG. 37, and in one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, and the like . . . . It is preferred, when the C moiety is present, that it be present in a concentration of from about 1 to about 90 mole percent, based upon the total number of moles of the A moiety and/or the B moiety and C moiety in the composition.”

In one embodiment, the aforementioned moiety A is preferably comprised of a magnetic element selected from the group consisting of a transition series metal, a rare earth series metal, or actinide metal, a mixture thereof, and/or an alloy thereof. In one embodiment, the moiety A is iron. In another embodiment, moiety A is nickel. In yet another embodiment, moiety A is cobalt. In yet another embodiment, moiety A is gadolinium. In another embodiment, the A moiety is selected from the group consisting of samarium, holmium, neodymium, and one or more other member of the Lanthanide series of the periodic table of elements.

In one preferred embodiment, two or more A moieties are present, as atoms, in one aspect of this embodiment. In one aspect of this embodiment, the magnetic susceptibilities of the atoms so present are both positive.

In one embodiment, two or more A moieties are present, at least one of which is iron. In one aspect of this embodiment, both iron and cobalt atoms are present.

When both iron and cobalt are present, it is preferred that from about 10 to about 90 mole percent of iron be present by mole percent of total moles of iron and cobalt present in the ABC moiety. In another embodiment, from about 50 to about 90 mole percent of iron is present. In yet another embodiment, from about 60 to about 90 mole percent of iron is present. In yet another embodiment, from about 70 to about 90 mole percent of iron is present.

In one preferred embodiment, moiety A is selected from the group consisting of iron, nickel, cobalt, alloys thereof, and mixtures thereof.

The moiety A may be present in the nanomagnetic material either in its elemental form, as an alloy, in a solid solution, or as a compound.

In one embodiment, it is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.)

In one embodiment, the nanomagnetic material has the formula A₁A₂(B)ₓC₁(C₂)_{y}, wherein each of A₁ and A₂ are separate magnetic A moieties, as described above; B is as defined elsewhere in this specification; x is an integer from 0 to 1; each of C₁ and C₂ is as descried elsewhere in this specification; and y is an integer from 0 to 1.

In this embodiment, there are always two distinct A moieties, such as, e.g., nickel and iron, iron and cobalt, etc. The A moieties may be present in equimolar amounts; or they may be present in non-equimolar amount.

In one aspect of this embodiment, either or both of the A₁ and A₂ moieties are radioactive. Thus, e.g., either or both of the A₁ and A₂ moieties may be selected from the group consisting of radioactive cobalt, radioactive iron, radioactive nickel, and the like. These radioactive isotopes are well known. Reference may be had, e.g., to U.S. Pat. Nos. 3,894,584; 3,936,440 (method of labeling complex metal chelates with radioactive metal isotopes); U.S. Pat. Nos. 4,031,387; 4,282,092; 4,572,797;4,642,193; 4,659,512; 4,704,245; 4,758,874 (minimization of radioactive material deposition in water-cooled nuclear reactors); U.S. Pat. No. 4,950,449 (inhibition of radioactive cobalt deposition); U.S. Pat. No. 4,647,585 (method for separating cobalt, nickel, and the like from alloys), U.S. Pat. Nos. 4,759,900; 4,781,198 (biopsy tracer needle); U.S. Pat. Nos. 4,876,449; 5,035,858; 5,196,113; 5,205,167; 5,222,065; 5,241,060 (base moiety-labeled detectable nucleotide); U.S. Pat. No. 6,314,153; and the like. The entire disclosure of each of these United States patents is hereby incorporated by reference into this specification.

In one preferred embodiment, at least one of the A₁ and A₂ moieties is radioactive cobalt. This radioisotope is discussed, e.g., in U.S. Pat. No. 3,936,440, the entire disclosure of which is hereby incorporated by reference into this specification.

In one embodiment, at least one of the A₁ and A₂ is radioactive iron. This radioisotope is also well known as is evidenced, e.g., by U.S. Pat. No. 4,459,356, the entire disclosure of which is also hereby incorporated by reference into this specification. Thus, and as is disclosed in such patent, “In accordance with the present invention, a radioactive stain composition is developed as a result of introduction of a radionuclide (e.g., radioactive iron isotope 59 Fe, which is a strong gamma emitter having peaks of 1.1 and 1.3 MeV) into BPS to form ferrous BPS . . . . In order to prepare the radioactive stain composition, sodium bathophenanthroline sulfonate (BPS), ascorbic acid and Tris buffer salts were obtained from Sigma Chemical Co. (St. Louis, Mo.). Enzymes grade acrylamide, N,N′methylenebisacrylamide and N,N,N′,N′-tetramethylethylenediamine (TEMED) are products of and were obtained from Eastman Kodak Co. (Rochester, N.Y.). Sodium dodecylsulfate (SDS) was obtained from Pierce Chemicals (Rockford, Ill.). The radioactive isotope (59 FeCl3 in 0.05M HCl, specific activity 15.6 mC/mg) was purchased from New England Nuclear (Boston, Mass.), but was diluted to 10 ml with 0.5N HCl to yield an approximately 0.1 mM Fe(III) solution.”

In the nanomagnetic particles, there may be, but need not be, a B moiety (such as, e.g., aluminum). There preferably are at least two C moieties such as, e.g., oxygen and nitrogen. The A moieties, in combination, preferably comprise at least about 80 mole percent of such a composition; and they more preferably comprise at least 90 mole percent of such composition.

When two C moieties are present, and when the two C moieties are oxygen and nitrogen, they preferably are present in a mole ratio such that from about 10 to about 90 mole percent of oxygen is present, by total moles of oxygen and nitrogen. It is preferred that at least about 60 mole percent of oxygen be present. In one embodiment, at least about 70 mole percent of oxygen is so present. In yet another embodiment, at least 80 mole percent of oxygen is so present.

One may measure the “surface oxygen content” of the surface of the nanomagnetic material, measuring the first 8.5 nanometers of material. In one embodiment, when such surface is measured, it is preferred that at least 50 mole percent of oxygen, by total moles of oxygen and nitrogen, be present in such surface. It is preferred that at least about 60 mole percent of oxygen be present. In one embodiment, at least about 70 mole percent of oxygen is so present. In yet another embodiment, at least 80 mole percent of oxygen is so present.

Without wishing to be bound to any particular theory, applicants believe that the presence of two distinct A moieties in their composition, and two distinct C moieties (such as, e.g., oxygen and nitrogen), provide better magnetic properties for applicants' nanomagnetic materials.

The B moiety, in one embodiment, in whatever form it is present, is preferably nonmagnetic, i.e., it has a relative magnetic permeability of about 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like.

In one embodiment, the B moiety has a relative magnetic permeability that is about equal to 1 plus the magnetic susceptibility. The relative magnetic susceptibilities of silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, copper, cesium, cerium, hafnium, iodine, iridium, lanthanum, lithium, lutetium, manganese, molybdenum, potassium, sodium, strontium, praseodymium, rhenium, rhodium, rubidium, ruthenium, scandium, selenium, tantalum, technetium, tellurium, chromium, thallium, thorium, thulium, titanium, vanadium, zinc, yttrium, ytterbium, zirconium, and the like. Reference may be had, e.g., to pages E-118 through E 123 of the aforementioned CRC Handbook of Chemistry and Physics.

In one embodiment, the nanomagnetic particles may be represented by the formula AₓB_{y}C_{z} wherein x+y+z is equal to 1. In this embodiment the ratio of x/y is at least 0.1 and preferably at least 0.2; and the ratio of z/x is from 0.001 to about 0.5.

In one preferred embodiment, the B material is aluminum and the C material is nitrogen, whereby an AlN moiety is formed. Without wishing to be bound to any particular theory, applicants believe that aluminum nitride (and comparable materials) are both electrically insulating and thermally conductive, thus providing an excellent combination of properties for certain end uses.

In one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, elemental fluorine, elemental sulfur, elemental hydrogen, elemental helium, the elemental chlorine, elemental bromine, elemental iodine, elemental boron, elemental phosphorus, and the like. In one aspect of this embodiment, the C moiety is selected from the group consisting of elemental oxygen, elemental nitrogen, and mixtures thereof.

In one embodiment, the C moiety is chosen from the group of elements that, at room temperature, form gases by having two or more of the same elements combine. Such gases include, e.g., hydrogen, the halide gases (fluorine, chlorine, bromine, and iodine), inert gases (helium, neon, argon, krypton, xenon, etc.), etc.

In one embodiment, the C moiety is chosen from the group consisting of oxygen, nitrogen, and mixtures thereof. In one aspect of this embodiment, the C moiety is a mixture of oxygen and nitrogen, wherein the oxygen is present at a concentration from about 10 to about 90 mole percent, by total moles of oxygen and nitrogen.

It is preferred, when the C moiety (or moieties) is present, that it be present in a concentration of from about 1 to about 90 mole percent, based upon the total number of moles of the A moiety and/or the B moiety and the C moiety in the composition. In one embodiment, the C moiety is both oxygen and nitrogen.

The molar ratio of A/(A and B and C) generally is preferably from about 1 to about 99 molar percent and, preferably, from about 10 to about 90 molar percent. In one embodiment, such molar ratio is from about 30 to about 60 molar percent.

The molar ratio of B/(A plus B plus C) generally is from about 1 to about 99 mole percent and, preferably, from about 10 to about 40 mole percent.

The molar ratio of C/(A plus B plus C) generally is from about 1 to about 99 mole percent and, preferably, from about 10 to about 50 mole percent.

In one embodiment, the B moiety is added to the nanomagnetic A moiety, preferably with a B/A molar ratio of from about 5:95 to about 95:5. In one aspect of this embodiment, the resistivity of the mixture of the B moiety and the A moiety is from about 1 micro-ohm-cm to about 10,000 micro-ohm-cm.

In one particularly preferred embodiment, the A moiety is iron, the B moiety is aluminum, and the molar ratio of A/B is about 70:30; the resistivity of this mixture is about 8 micro-ohms-centimeters.

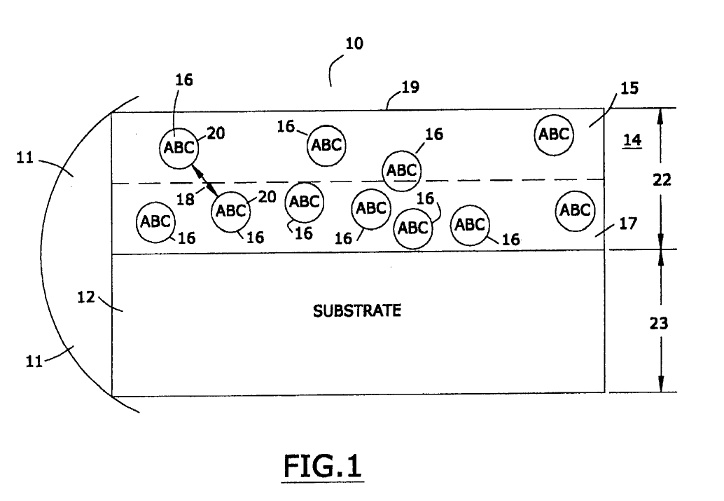

FIG. 1 is a schematic illustration, not drawn to scale, of a coated substrate assembly 10 comprised of a substrate 12 and, disposed thereon, a coating 14 comprised of a multiplicity of nanomagnetic particles 16. Similar coated substrate assemblies are illustrated and described in applicants' United States patents hereinbelow and elsewhere in this specification. Reference may be had, e.g., to U.S. Pat. No. 6,506,972 (magnetically shielded conductor), U.S. Pat. No. 6,700,472 (magnetic thin film inductors), U.S. Pat. No. 6,713,671 (magnetically shielded assembly), U.S. Pat. No. 6,765,144 (magnetic resonance imaging coated assembly), and the like. The entire disclosure of each of these United States patents is hereby incorporated by reference into this specification.

In the preferred embodiment illustrated in FIG. 1, it will be seen that the coating 14 is preferably comprised of a top half 15 and a bottom half 17, wherein a disproportionate amount (at least 60 weight percent) of the nanomagnetic particles 16 are preferably disposed in such bottom half 17. In one preferred embodiment, at least 70 percent of the nanomagnetic particles 16 are disposed in the bottom half 17.

In another embodiment, not shown, a disproportionate amount of the nanomagnetic particles are disposed in the top half 15 of the coating 14.

Without wishing to be bound to any particular theory, applicant's believe that having a nonhomogeneous distribution of the nanomagnetic particles in the coating 14 affords one the opportunity to change the path of energy passing through the coating 14.

Referring to FIG. 1, and to the preferred embodiment depicted therein, it will be seen that the nanomagnetic particles 16 are preferably comprised of the “ABC” atoms described elsewhere in this specification. With regard to such “ABC” particles, the term “coherence length” refers to the smallest distance 18 between the surfaces 20 of any particles 16 that are adjacent to each other. In one aspect of this embodiment, it is preferred that such coherence length, with regard to such ABC particles, be less than about 100 nanometers and, preferably, less than about 50 nanometers. In one embodiment, such coherence length is less than about 20 nanometers. It is preferred that, regardless of the coherence length used, it be at least 2 times as great as the maximum dimension of the particles 16.

The Mass Density of the Nanomagnetic Particles

In one embodiment, the nanomagnetic material preferably has a mass density of at least about 0.001 grams per cubic centimeter; in one aspect of this embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In another embodiment, the material has a mass density of at least about 3 grams per cubic centimeter. In another embodiment, the nanomagnetic material has a mass density of at least about 4 grams per cubic centimeter.

The Thickness of the Coating 14

Referring again to FIG. 1, and to the preferred embodiment depicted therein, the coating 14 may be comprised of one layer of material, two layers of material, or three or more layers of material. Regardless of the number of coating layers used, it is preferred that the total thickness 22 of the coating 14 be at least about 400 nanometers and, preferably, be from about 400 to about 4,000 nanometers. In one embodiment, thickness 22 is from about 600 to about 1,400 nanometers. In another embodiment, thickness 22 is from about 800 to about 1200 nanometers.

In the embodiment depicted, the substrate 12 has a thickness 23 that is substantially greater than the thickness 22. As will be apparent, the coated substrate 10 is not drawn to scale.

In one embodiment, the thickness 22 is preferably less than about 5 percent of thickness 23 and, more preferably, less than about 2 percent. In one embodiment, the thickness 22 is no greater than about 1.5 percent of the thickness 23.

The Flexibility of Coated Substrate 10

Referring to FIG. 1, and in one preferred embodiment thereof, substrate 12 is a conductor that preferably has a resistivity at 20 degrees Centigrade of from about 1 to about 100-microohom-centimeters. In this embodiment, disposed above the conductor 12 is a film 14 comprised of nanomagnetic particles 16 that preferably have a maximum dimension of from about 1 to about 100 nanometers. The film 14, in one embodiment, also preferably has a saturation magnetization of from about 200 to about 26,000 Gauss and a thickness of less than about 2 microns.

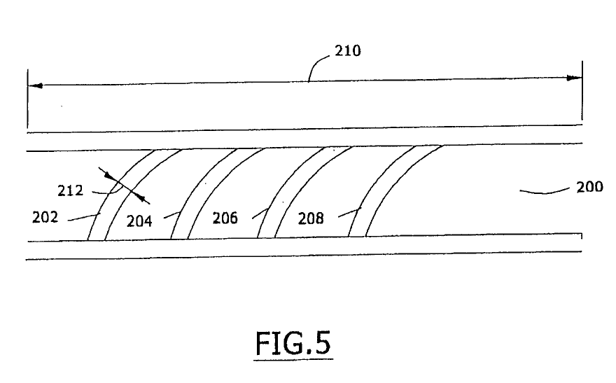

In one aspect of this embodiment, conductor assembly 10 is flexible, having a bend radius of less than 2 centimeters. Reference may be had, e.g., to U.S. Pat. No. 6,506,972, the entire disclosure of which is hereby incorporated by reference into this specification. A similar device is depicted in FIG. 5 of U.S. Pat. No. 6,713,671; the entire disclosure of such United States patent is hereby incorporated by reference into this specification.

As used in this specification, the term flexible refers to an assembly that can be bent to form a circle with a radius of less than 2 centimeters without breaking. Put another way, the bend radius of the coated assembly is preferably less than 2 centimeters. Reference may be had, e.g., to U.S. Pat. Nos. 4,705,353, 5,946,439, 5,315,365, 4,641,917, 5,913,005, and the like. The entire disclosure of each of these United States patents is hereby incorporated by reference into this specification.

Without wishing to be bound to any particular theory, applicants believe that the use of nanomagnetic particles in their coatings and their articles of manufacture allows one to produce a flexible device that otherwise could not be produced were not the materials so used nano-sized (less than 100 nanometers).

In another embodiment, not shown, the assembly 10 is not flexible.

The Morphological Density of the Coating 14

In one preferred embodiment, and referring to FIG. 1, the coating 14 has a morphological density of at least about 98 percent. In the embodiment depicted, the coating 14 has a thickness 22 of from about 400 to about 2,000 nanometers and, in one embodiment, has a thickness 22 of from about 600 to about 1200 nanometers.

As is known to those skilled in the art, the morphological density of a coating is a function of the ratio of the dense coating material on its surface to the pores on its surface; and it is usually measured by scanning electron microscopy. By way of illustration, e.g., published United States patent application U.S. 2003/0102222A1 contains a FIG. 3A that is a scanning electron microscope (SEM) image of a coating of “long” single-walled carbon nanotubes on a substrate. Referring to this SEM image, it will be seen that the white areas are the areas of the coating where pores occur.

The technique of making morphological density measurements also is described, e.g., in a M. S. thesis by Raymond Lewis entitled “Process study of the atmospheric RF plasma deposition system for oxide coatings” that was deposited in the Scholes Library of Alfred University, Alfred, New York in 1999 (call Number TP2 a75 1999 vol 1, no. 1.).

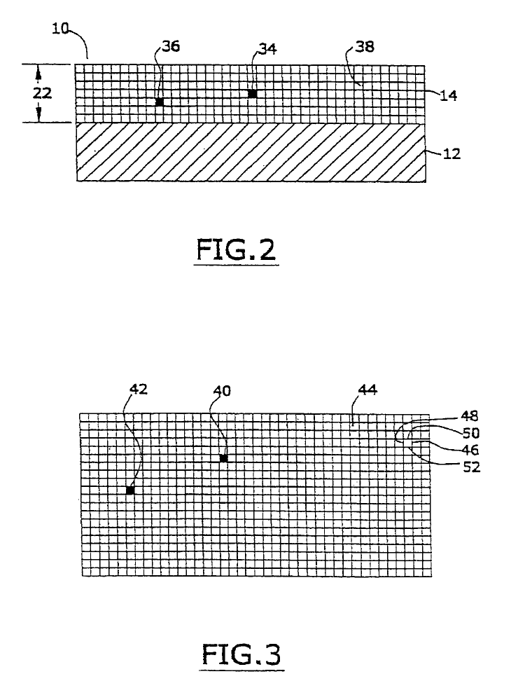

The scanning electron microscope (SEM) images obtained in making morphological density measurements can be divided into a matrix., as is illustrated in FIGS. 2 and 3 which schematically illustrate the porosity of the side of coating 14, and the top of the coating 14. The SEM image depicted shows two pores 34 and 36 in the cross-sectional area 38, and it also shows two pores 40 and 42 in the top 44. As will be apparent, the SEM image can be divided into a matrix whose adjacent lines 46/48, and adjacent lines 50/52 define a square portion with a surface area of 100 square nanometers (10 nanometers×10 nanometers). Each such square portion that contains a porous area is counted, as is each such square portion that contains a dense area. The ratio of dense areas/porous areas,×100, is preferably at least 98. Put another way, the morphological density of the coating 14 is at least 98 percent. In one embodiment, the morphological density of the coating 14 is at least about 99 percent. In another embodiment, the morphological density of the coating 14 is at least about 99.5 percent.

One may obtain such high morphological densities by atomic size deposition, i.e., the particles sizes deposited on the substrate are atomic scale. The atomic scale particles thus deposited often interact with each other to form nano-sized moieties that are less than 100 nanometers in size.

The Surface Roughness of the Coating 14

In one embodiment, the coating 14 (see FIG. 1) has an average surface roughness of less than about 100 nanometers and, more preferably, less than about 10 nanometers. As is known to those skilled in the art, the average surface roughness of a thin film is preferably measured by an atomic force microscope (AFM). Reference may be had, e.g., to U.S. Pat. No. 5,420,796 (method of inspecting planarity of wafer surface), U.S. Pat. Nos. 6,610,004, 6,140,014, 6,548,139, 6,383,404, 6,586,322, 5,832,834, and 6,342,277. The entire disclosure of each of these United States patents is hereby incorporated by reference into this specification.

Alternatively, or additionally, one may measure surface roughness by a laser interference technique. This technique is well known. Reference may be had, e.g., to U.S. Pat. No. 6,285,456 (dimension measurement using both coherent and white light interferometers), U.S. Pat. Nos. 6,136,410, 5,843,232 (measuring deposit thickness), U.S. Pat. No. 4,151,654 (device for measuring axially symmetric aspherics), and the like. The entire disclosures of these United States patents are hereby incorporated by reference into this specification.

Hydrophobic and Hydrophilic Coatings

By varying the surface roughness of the coating 14 (see FIG. 1), one may make the surface 19 of such coating either hydrophobic or hydrophilic.

As is known to those skilled in the art, a hydrophobic material is antagonistic to water and incapable of dissolving in water. Inasmuch as the average water droplet has a minimum cross-sectional dimension of at least about 3 nanometers, the water droplets will tend not to bond to a coated surface 19 which, has a surface roughness of, e.g., 1 nanometer.

One may vary the average surface roughness of coated surface 19 by varying the pressure used in the sputtering process described elsewhere in this specification. In general, the higher the gas pressure used, the rougher the surface.

If, on the other hand, one modifies the sputtering process to allow a surface roughness of at about, e.g., 20 nanometers, the water droplets then have an opportunity to bond to the surface 19 which, in this embodiment, will tend to be hydrophilic.

Durable Properties of the Coated Substrate 10

In one embodiment, the coated substrate of this invention has durable magnetic properties that do not vary upon extended exposure to a saline solution. If the magnetic moment of a coated substrate is measured at “time zero” (i.e., prior to the time it has been exposed to a saline solution), and then the coated substrate is then immersed in a saline solution comprised of 7.0 mole percent of sodium chloride and 93 mole percent of water, and if the substrate/saline solution is maintained at atmospheric pressure and at temperature of 98.6 degrees Fahrenheit for 6 months, the coated substrate, upon removal from the saline solution and drying, will be found to have a magnetic moment that is within plus or minus 5 percent of its magnetic moment at time zero.

In another embodiment, the coated substrate of this invention has durable mechanical properties when tested by the saline immersion test described above.

Thus, e.g., the substrate 12, prior to the time it is coated with coating 14, has a certain flexural strength, and a certain spring constant.

The flexural strength is the strength of a material in bending, i.e., its resistance to fracture. As is disclosed in ASTM C-790, the flexural strength is a property of a solid material that indicates its ability to withstand a flexural or transverse load. As is known to those skilled in the art, the spring constant is the constant of proportionality k which appears in Hooke's law for springs. Hooke's law states that: F=−kx, wherein F is the applied force and x is the displacement from equilibrium. The spring constant has units of force per unit length.

Means for measuring the spring constant of a material are well known to those skilled in the art. Reference may be had, e.g., to U.S. Pat. No. 6,360,589 (device and method for testing vehicle shock absorbers), U.S. Pat. No. 4,970,645 (suspension control method and apparatus for vehicle), U.S. Pat. Nos. 6,575,020, 4,157,060, 3,803,887, 4,429,574, 6,021,579, and the like. The entire disclosure of each of these United States patents is hereby incorporated by reference into this specification.

Referring again to FIG. 1, the flexural strength of the uncoated substrate 10 preferably differs from the flexural strength of the coated substrate 10 by no greater than about 5 percent. Similarly, the spring constant of the uncoated substrate 10 differs from the spring constant of the coated substrate 10 by no greater than about 5 percent.

In one embodiment, the coating 14 is biocompatible with biological organisms. As used herein, the term biocompatible refers to a coating whose chemical composition does not change substantially upon exposure to biological fluids. Thus, when the coating 14 is immersed in a 7.0 mole percent saline solution for 6 months maintained at a temperature of 98.6 degrees Fahrenheit, its chemical composition (as measured by, e.g., energy dispersive X-ray analysis [EDS, or EDAX]) is substantially identical to its chemical composition at “time zero.”

The Susceptibility of the Coated Substrate 10

In one preferred embodiment (see FIG. 1), the coated substrate 10 has a direct current (d.c.) magnetic susceptibility within a specified range. As is known to those skilled in the art, magnetic susceptibility is the ratio of the magnetization of a material to the magnetic field strength; it is a tensor when these two quantities are not parallel; otherwise it is a simple number. Reference may be had, e.g., to U.S. Pat. No. 3,614,618 (magnetic susceptibility tester), U.S. Pat. No. 3,644,823 (nulling coil for magnetic susceptibility logging), U.S. Pat. No. 3,758,848 (method and system with voltage cancellation for measuring the magnetic susceptibility of a subsurface earth formation), U.S. Pat. No. 3,879,658 (apparatus for measuring magnetic susceptibility), U.S. Pat. No. 3,980,076 (method for measuring externally of the human body magnetic susceptibility changes), U.S. Pat. No. 4,277,750 (induction probe for the measurement of magnetic susceptibility), U.S. Pat. No. 4,662,359 (use of magnetic susceptibility probes in the treatment of cancer), U.S. Pat. No. 4,985,165 (material having a predeterminable magnetic susceptibility), U.S. Pat. No. 5,300,886 (method to enhance the susceptibility of MRI for magnetic susceptibility effects), U.S. Pat. No. 6,208,884 (noninvasive room temperature instrument to measure magnetic susceptibility variations in body tissue), U.S. Pat. No. 6,477,398 (resonant magnetic susceptibility imaging), and the like. The entire disclosure of each of these United States patents is hereby incorporated by reference into this specification.

In one aspect of this embodiment, and referring again to FIG. 1, the substrate 12 is a stent that is comprised of wire mesh constructed in such a manner as to define a multiplicity of openings. The mesh material is preferably a metal or metal alloy, such as, e.g., stainless steel, Nitinol (an alloy of nickel and titanium), niobium, copper, etc.

Typically the materials used in stents tend to cause current flow when exposed to a radio frequency field. When the field is a nuclear magnetic resonance field, it generally has a direct current component, and a radio-frequency component. For MRI (magnetic resonance imaging) purposes, a gradient component is added for spatial resolution.

The material or materials used to make the stent itself have certain magnetic properties such as, e.g., magnetic susceptibility. Thus, e.g., niobium has a magnetic susceptibility of 1.95×10⁻⁶ centimeter-gram-second units. Nitonol has a magnetic susceptibility of from about 2.5 to about 3.8×10⁻⁶ centimeter-gram-second units. Copper has a magnetic susceptibility of from −5.46 to about −6.16×10⁻⁶ centimeter-gram-second units.

The total magnetic susceptibility of an object is equal to the mass of the object times its susceptibility. Thus, assuming an object has equal parts of niobium, Nitinol, and copper, its total susceptibility would be equal to (+1.95+3.15−5.46)×10⁻⁶ cgs, or about 0.36×10⁻⁶ cgs.

In a more general case, where the masses of niobium, Nitinol, and copper are not equal in the object, the susceptibility, in c.g.s. units, would be equal to 1.95 Mn+3.15 Mni−5.46Mc, wherein Mn is the mass of niobium, Mni is the mass of Nitinol, and Mc is the mass of copper.

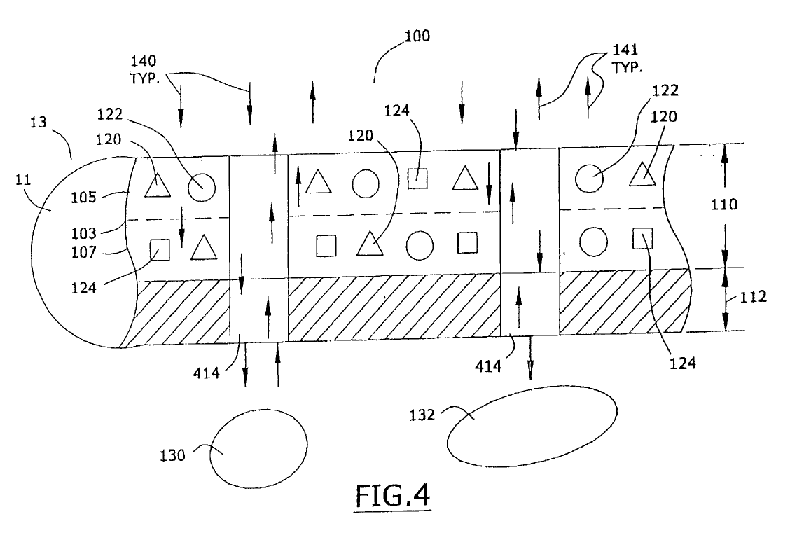

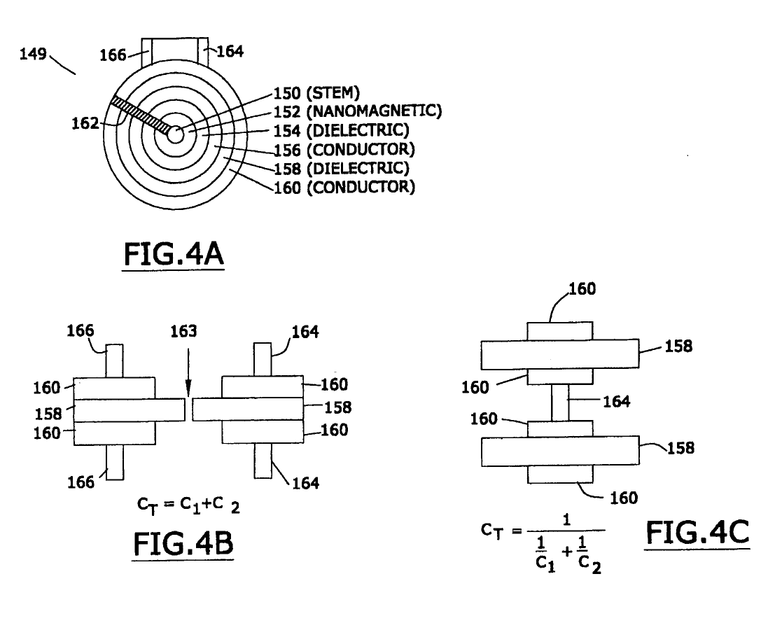

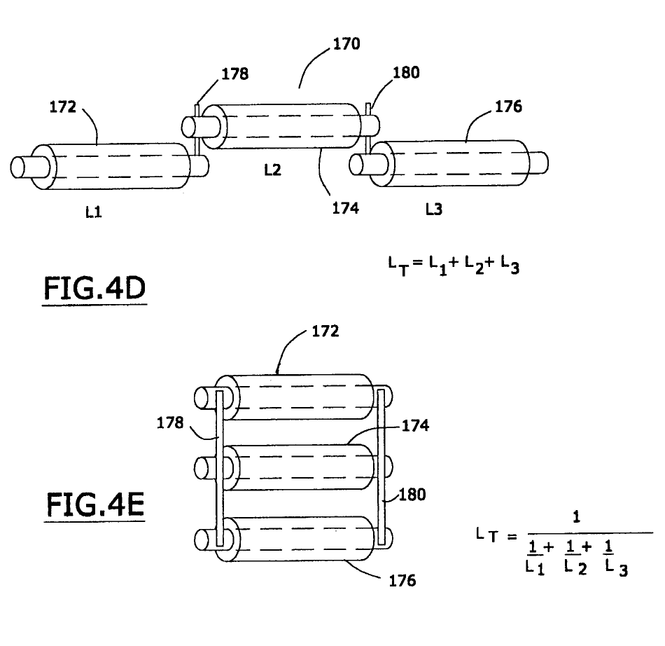

Referring again to FIG. 1, and in one preferred embodiment thereof, the coated substrate assembly 10 preferably materials that will provide the desired mechanical properties generally do not have desirable magnetic and/or electromagnetic properties. In an ideal situation, and referring to FIG. 4, the stent 100 will produce substantially no loop currents and substantially no surface eddy currents when exposed to magnetic resonance imaging (MRI) radiation and, in such situation, has an effective zero magnetic susceptibility. Put another way, ideally the direct current magnetic susceptibility of an ideal coated substrate that is exposed to MRI radiation should be about 0.

A d.c. (“direct current”) magnetic susceptibility of precisely zero is often difficult to obtain. In general, and referring again to FIG. 1, it is sufficient if the direct current susceptibility of the coated substrate 10 is plus or minus 1×10⁻³ centimeter-gram-seconds (cgs) and, more preferably, plus or minus 1×10⁻⁴ centimeter-gram-seconds. In one embodiment, the d.c. susceptibility of the coated substrate 10 is equal to plus or minus 1×10⁻⁵ centimeter-gram-seconds. In another embodiment, the d.c. susceptibility of the coated substrate 10 is equal to plus or minus 1×10⁻⁶ centimeter-gram-seconds.

In one embodiment, and referring again to FIG. 1, the coated substrate assembly 10 is in contact with biological tissue 11. In FIG. 1, only a portion of the biological tissue 11 actually contiguous with assembly 10 is shown for the sake of simplicity of representation. In such an embodiment, it is preferred that such biological tissue 11 be taken into account when determining the net susceptibility of the assembly, and that such net susceptibility of the assembly 10 in contact with bodily tissue 11 is plus or minus plus or minus 1×10⁻³ centimeter-gram-seconds (cgs), or plus or minus 1×10⁻⁴ centimeter-gram-seconds, or plus or minus 1×10⁻⁵ centimeter-gram-seconds, or plus or minus 1×10⁻⁶ centimeter-gram-seconds. In this embodiment, the materials comprising the nanomagnetic coating 14 on the substrate 12 are chosen to have susceptibility values that, in combination with the susceptibility values of the other components of the assembly, and of the bodily fluid, will yield the desired values.

The prior art has heretofore been unable to provide such an implantable stent 100 (see FIG. 4) that will have the desired degree of net magnetic susceptibility. Applicants' invention allows one to compensate for the deficiencies of the current stents, and/or of the current stents in contact with bodily fluid, by canceling the undesirable effects due to their magnetic susceptibilities, and/or by compensating for such undesirable effects.

When different objects are subjected to an electromagnetic field (such as a MRI field), they will exhibit different magnetic responses at different field strengths. Thus, e.g., copper, at a d.c. field strength of 1.5 Tesla, changes its magnetization as a function of the composite field strength (including the d.c. field strength, the r.f. field strength, and the gradient field strength) at a rate (defined by delta-magnetization/delta composite field strength) that is decreasing. With regard to the r.f. field and the gradient field, it should be understood that the order of magnitude of these fields is relatively small compared to the d.c. field, which is usually about 1.5 Tesla. The slope of the graph of magnetization versus field strength for copper is negative; this negative slope indicates that copper, in response to the applied fields, is opposing the applied fields. Because the applied fields (including r.f. fields, and the gradient fields), are required for effective MRI imaging, the response of the copper to the applied fields tends to block the desired imaging. The d.c. susceptibility of copper is equal to the mass of the copper present in the device 10 times its magnetic susceptibility.

By comparison to copper, the ideal magnetization response of a composite assembly (such as, e.g., assembly 100/11) will be a line whose slope is substantially zero. As used herein, the term “substantially zero” includes a slope will produce an effective magnetic susceptibility of from about 1×10⁻⁷ to about 1×10⁻⁸ centimeters-gram-second (cgs).

One means of correcting negative slope in the graph for copper is by coating the copper with a coating which produces a magnetization response with a positive slope so that the composite material produces the desired effective magnetic susceptibility of from about 1×10⁻⁷ to about 1×10⁻⁸ centimeters-gram-second (cgs) units. In order to do so, the following equation must be satisfied: (magnetic susceptibility of the uncoated device) (mass of uncoated device)+(magnetic susceptibility of copper)(mass of copper)=from about 1×10⁻⁷ to about 1×10⁻⁸ centimeters-gram-second (cgs).

In one embodiment, the desired correction for the slope of the copper graph may be obtained by coating the copper with a coating comprised of both nanomagnetic material and nanodielectric material.

In one aspect of this embodiment, the nanomagnetic material preferably has an average particle size of less than about 20 nanometers and a saturation magnetization of from 10,000 to about 26,000 Gauss. In another aspect of this embodiment, the nanomagnetic material used is iron. In another aspect of this embodiment, the nanomagnetic material used is FeAlN. In yet another aspect of this embodiment, the nanomagnetic material is FeAl. Other suitable materials will be apparent to those skilled in the art and include, e.g., nickel, cobalt, magnetic rare earth materials and alloys, thereof, and the like.

In this embodiment, the nanodielectric material used preferably has a resistivity at 20 degrees Centigrade of from about 1×10⁻⁵ ohm-centimeters to about 1×10¹³ ohm-centimeters.

Referring to FIG. 4, and in the preferred embodiment depicted therein, a coated stent assembly 100 that is comprised of a stent 104 on which is disposed a coating 103 is illustrated. The coating 103 is comprised of nanomagnetic material 120 that is preferably inhomogeneously dispersed within nanodielectric material 122, which acts as an insulating matrix. In general, the amount of nanodielectric material 122 in coating 103 exceeds the amount of nanomagnetic material 120 in such coating 103.

In one embodiment, the coating 103 is comprised of at least about 70 mole percent of such nanodielectric material (by total moles of nanomagnetic material and nanodielectric material). In another embodiment, the coating 103 is comprised of less than about 20 mole percent of the nanomagnetic material 120, by total moles of nanomagnetic material and nanodielectric material. In one embodiment, the nanodielectric material used is aluminum nitride.

Referring again to FIG. 4, one may optionally include nanoconductive material 124 in the coating 103. This nanoconductive material 124 generally has a resistivity at 20 degrees Centigrade of from about 1×10⁻⁶ ohm-centimeters to about 1×10⁻⁵ ohm-centimeters; and it generally has an average particle size of less than about 100 nanometers. In one aspect of this embodiment, the nanoconductive material used is aluminum.

Referring again to FIG. 4, and in the embodiment depicted, it will be seen that two layers 105/107 are preferably used to obtain the desired correction. In one embodiment, three or more such layers are used. Regardless of the number of such layers 105/107 used, it is preferred that the thickness 110 of coating 103 be from about 400 to about 4000 nanometers. In one aspect of this embodiment, at least about 60 weight percent of the nanomagnetic material 120 is disposed in layer 107.

In the embodiment depicted in FIG. 4, the direct current susceptibility of the assembly depicted is equal to the sum of the (mass)×(susceptibility) for each individual layer 105/107 and for the substrate 104.

As will be apparent, it may be difficult with only one layer of coating material to obtain the desired correction for the material comprising the stent assembly 100. With a multiplicity of layers comprising the coating 103, which may have the same and/or different thicknesses, and/or the same and/or different masses, and/or the same and/or different compositions, and/or the same and/or different magnetic susceptibilities, more flexibility is provided in obtaining the desired correction.

Without wishing to be bound to any particular theory, applicants believe that, in the assembly 100 depicted in FIG. 4, each of the different species 120/122/124 within the coatings 105/107 retains its individual magnetic characteristics. These species are preferably not alloyed with each other; when such species are alloyed with each other, each of the species does not retain its individual magnetic characteristics.

An alloy, as that term is used in this specification, is a substance having magnetic properties and consisting of two or more elements, which usually are metallic elements. The bonds in the alloy are usually metallic bonds, and thus the individual elements in the alloy do not retain their individual magnetic properties because of the substantial “crosstalk” between the elements via the metallic bonding process.

By comparison, e.g., materials that are covalently bond to each other are more likely to retain their individual magnetic characteristics; it is such materials whose behavior is illustrated in FIG. 4. Each of the “magnetically distinct” materials may be, e.g., a material in elemental form, a compound, an alloy, etc.

In one embodiment, and referring again to FIG. 4, one may mix “positively magnetized materials” with “negatively magnetized materials” to obtain the desired degree of net magnetization. As is known to those skilled in the art, the positively magnetized species include, e.g., those species that exhibit paramagnetism, superparamagnetism, ferromagnetism, and/or ferrimagnetism.

Paramagnetism is a property exhibited by substances which, when placed in a magnetic field, are magnetized parallel to the field to an extent proportional to the field (except at very low temperatures or in extremely large magnetic fields). Paramagnetic materials are well known to those skilled in the art. Reference may be had, e.g., to U.S. Pat. No. 5,578,922 (paramagnetic material in solution), U.S. Pat. No. 4,704,871 (magnetic refrigeration apparatus with belt of paramagnetic material), U.S. Pat. No. 4,243,939 (base paramagnetic material containing ferromagnetic impurity), U.S. Pat. No. 3,917,054 (articles of paramagnetic material), U.S. Pat. No. 3,796,4999 (paramagnetic material disposed in a gas mixture), and the like. The entire disclosure of each of these United States patents is hereby incorporated by reference into this specification.

Superparamagnetic materials are also well known to those skilled in the art. Reference may be had, e.g., to U.S. Pat. No. 5,238,811, the entire disclosure of which is hereby incorporated by reference into this specification, it is disclosed (at column 5) that: “In one embodiment, the superparamagnetic material used is a substance which has a particle size smaller than that of a ferromagnetic material and retains no residual magnetization after disappearance of the external magnetic field. The superparamagnetic material and ferromagnetic material are quite different from each other in their hysteresis curve, susceptibility, Mesbauer effect, etc. Indeed, ferromagnetic materials are most suited for the conventional assay methods since they require that magnetic micro-particles used for labeling be efficiently guided even when a weak magnetic force is applied.

The preparation of these superparamagnetic materials is discussed at columns 6 et seq. of U.S. Pat. No. 5,238,811, wherein it is disclosed that: “The ferromagnetic substances can be selected appropriately, for example, from various compound magnetic substances such as magnetite and gamma-ferrite, metal magnetic substances such as iron, nickel and cobalt, etc. The ferromagnetic substances can be converted into ultramicro particles using conventional methods excepting a mechanical grinding method, i.e., various gas phase methods and liquid phase methods. For example, an evaporation-in-gas method, a laser heating evaporation method, a coprecipitation method, etc. can be applied. The ultramicro particles produced by the gas phase methods and liquid phase methods contain both superparamagnetic particles and ferromagnetic particles in admixture, and it is therefore necessary to separate and collect only those particles which show superparamagnetic property. For the separation and collection, various methods including mechanical, chemical and physical methods can be applied, examples of which include centrifugation, liquid chromatography, magnetic filtering, etc. The particle size of the superparamagnetic ultramicro particles may vary depending upon the kind of the ferromagnetic substance used but it must be below the critical size of single domain particles. Preferably, it is not larger than 10 nm when the ferromagnetic substance used is magnetite or gamma-ferrite and it is not larger than 3 nm when pure iron is used as a ferromagnetic substance, for example.”

Ferromagnetic materials may also be used as the positively magnetized species. As is known to those skilled in the art, ferromagnetism is a property, exhibited by certain metals, alloys, and compounds of the transition (iron group), rare-earth, and actinide elements, in which the internal magnetic moments spontaneously organize in a common direction; this property gives rise to a permeability considerably greater than that of a cuum, and also to magnetic hysteresis. Reference may be had, e.g., to U.S. Pat. Nos. 6,475,650; 6,299,990; 6,690,287 (ferromagnetic material having improved impedance matching); U.S. Pat. No. 6,366,083 (crud layer containing ferromagnetic material on nuclear fuel rods); U.S. Pat. No. 6,011,674 (magnetoresistance effect multilayer film with ferromagnetic film sublayers of different ferromagnetic material compositions); U.S. Pat. No. 5,648,015 (process for preparing ferromagnetic materials); U.S. Pat. Nos. 5,382,304; 5,272,238 (organo-ferromagnetic material); U.S. Pat. No. 5,247,054 (organic polymer ferromagnetic material); U.S. Pat. No. 5,030,371 (acicular ferromagnetic material consisting essentially of iron-containing chromium dioxide); U.S. Pat. No. 4,917,736 (passive ferromagnetic material); U.S. Pat. No. 4,863,715 (contrast agent comprising particles of ferromagnetic material); U.S. Pat. No. 4,835,510 (magnetoresistive element of ferromagnetic material); U.S. Pat. No. 4,739,294 (amorphous and non-amorphous ferromagnetic material); U.S. Pat. No. 4,289,937 (fine grain ferromagnetic material); U.S. Pat. No. 4,023,412 (the Curie point of a ferromagnetic material); U.S. Pat. No. 4,015,030 (stabilized ferromagnetic material); U.S. Pat. No. 4,004,997 (a polymerizable composition containing a magnetized powdered ferromagnetic material); U.S. Pat. No. 3,851,375 (sintered oxidic ferromagnetic material); U.S. Pat. No. 3,850,706 (ferromagnetic materials comprised of transition metals); and the like. The entire disclosure of each of these United States patents is hereby incorporated by reference into this specification.

Ferrimagnetic materials may also be used as the positively magnetized specifies. As is known to those skilled in the art, ferrimagnetism is a type of magnetism in which the magnetic moments of neighboring ions tend to align nonparallel, usually antiparallel, to each other, but the moments are of different magnitudes, so there is an appreciable, resultant magnetization. Reference may be had, e.g., to U.S. Pat. Nos. 6,538,919; 6,056,890 (ferrimagnetic materials with temperature stability); U.S. Pat. Nos. 4,649,495; 4,062,920 (lithium-containing ferrimagnetic materials); U.S. Pat. Nos. 4,059,664; 3,947,372 (ferromagnetic material); U.S. Pat. No. 3,886,077 (garnet structure ferromagnetic material); U.S. Pat. Nos. 3,765,021; 3,670,267; and the like. The entire disclosure of each of these United States patents is hereby incorporated by reference into this specification.

By way of yet further illustration, and not limitation, some suitable positively magnetized species include, e.g., iron; iron/aluminum; iron/aluminum oxide; iron/aluminum nitride; iron/tantalum nitride; iron/tantalum oxide; nickel; nickel/cobalt; cobalt/iron; cobalt; samarium; gadolinium; neodymium; mixtures thereof; nano-sized particles of the aforementioned mixtures, where super-paramagnetic properties are exhibited; and the like.

By way of yet further illustration, other suitable positively magnetized species are listed in the “CRC Handbook of Chemistry and Physics,” 63^{rd} Edition (CRC Press, Inc., Boca-Raton, Fla., 1982-1983). As is discussed on pages E-118 to E-123 of such CRC Handbook, materials with positive susceptibility include, e.g., aluminum, americium, cerium (beta form), cerium (gamma form), cesium, compounds of cobalt, dysprosium, compounds of dysprosium, europium, compounds of europium, gadolium, compounds of gadolinium, hafnium, compounds of holmium, iridium, compounds of iron, lithium, magnesium, manganese, molybdenum, neodymium, niobium, osmium, palladium, plutonium, potassium, praseodymium, rhodium, rubidium, ruthenium, samarium, sodium, strontium, tantalum, technicium, terbium, thorium, thulium, titanium, tungsten, uranium, vanadium, ytterbium, yttrium, and the like.

In addition to using positively magnetized species in coating 103 (see FIG. 4), one may also use negatively magnetized species. The negatively magnetized species include those materials with negative susceptibilities that are listed on such pages E-118 to E-123 of the CRC Handbook. By way of illustration and not limitation, such species include, e.g.: antimony; argon; arsenic; barium; beryllium; bismuth; boron; calcium; carbon; chromium; copper; gallium; germanium; gold; indium; krypton; lead; mercury; phosphorous; selenium; silicon; silver; sulfur; tellurium; thallium; tin (gray); xenon; zinc; and the link.

Many diamagnetic materials also are suitable negatively magnetized species. As is known to those skilled in the art, diamagnetism is that property of a material that is repelled by magnets. The term “diamagnetic susceptibility” refers to the susceptibility of a diamagnetic material, which is always negative. Diamagnetic materials are well known to those skilled in the art. Reference may be had, e.g., to U.S. Pat. No. 6,162,364 (diamagnetic objects); U.S. Pat. No. 6,159,271 (diamagnetic liquid); U.S. Pat. No. 5,408,178 (diamagnetic and paramagnetic objects); U.S. Pat. No. 5,315,997 (method of magnetic resonance imaging using diamagnetic contrast); U.S. Pat. Nos. 5,162,301; 5,047,392 (diamagnetic colloids); U.S. Pat. Nos. 5,043,101; 5,026,681 (diamagnetic colloid pumps); U.S. Pat. No. 4,908,347 (diamagnetic flux shield); U.S. Pat. Nos. 4,778,594; 4,735,796; 4,590,922; 4,290,070; 3,899,758; 3,864,824; 3,815,963 (pseudo-diamagnetic suspension); U.S. Pat. Nos. 3,597,022; 3,572,273; and the like. The entire disclosure of each of these United States patents is hereby incorporated by reference into this specification.

By way of further illustration, the diamagnetic material used may be an organic compound with a negative susceptibility. Referring to pages E-123 to pages E-134 of the aforementioned CRC Handbook, such compounds include, e.g.: alanine; allyl alcohol; amylamine; aniline; asparagines; aspartic acid; butyl alcohol; chloresterol; coumarin; diethylamine; erythritol; eucalyptol; fructose; galactose; glucose; D-glucose; glutamic acid; glycerol; glycine; leucine; isoleucine; mannitol; mannose; and the like.

Referring again to FIG. 4, when a positively magnetized species is mixed with a negatively magnetized species, and assuming that each species retains its magnetic properties, the resulting magnetic properties exhibit substantially zero magnetization. In this embodiment, one must insure that the positively magnetized species does not lose its magnetic properties, as often happens when one material is alloyed with another. The magnetic properties of alloys and compounds containing different species are known, and thus it readily ascertainable whether the different species that make up such alloys and/or compounds have retained their unqiue magnetic characteristics.

Without wishing to be bound to any particular theory, applicants believe that, when a positively magnetized species is mixed with a negatively magnetized species, and assuming that each species retains its magnetic properties, the desired magnetization plot (substantially zero slope) will be achieved when the volume of the positively magnetized species times its positive susceptibility is substantially equal to the volume of the negatively magnetized species times its negative susceptibility for this relationship to hold, however, each of the positively magnetized species and the negatively magnetized species must retain the distinctive magnetic characteristics when mixed with each other.

Thus, for example, if element A has a positive magnetic susceptibility, and element B has a negative magnetic susceptibility, the alloying of A and B in equal proportions may not yield a zero magnetization compact.

Without wishing to be bound to any particular theory, nano-sized particles, or micro-sized particles (with a size of at least about 0.5 nanometers) tend to retain their magnetic properties as long as they remain in particulate form. On the other hand, alloys of such materials often do not retain such properties.

Nullification of the Susceptibility Contribution Due to the Substrate

As will be apparent by reference, e.g., to FIG. 4, when the substrate 104 is a copper stent, the copper substrate 104 depicted therein has a negative susceptibility, the coating 103 depicted therein preferably has a positive susceptibility, and the coated substrate 100 thus has a substantially zero susceptibility. As will also be apparent, some substrates (such niobium, nitinol, stainless steel, etc.) have positive susceptibilities. In such cases, and in one preferred embodiment, the coatings should preferably be chosen to have a negative susceptibility so that, under the conditions of the MRI radiation (or of any other radiation source used), the net susceptibility of the coated object is still substantially zero. As will be apparent, the contribution of each of the materials in the coating(s) is a function of the mass of such material and its magnetic susceptibility.

The magnetic susceptibilities of various substrate materials are well known. Reference may be had, e.g., to pages E-118 to E-123 of the “Handbook of Chemistry and Physics,” 63rd edition (CRC Press, Inc., Boca Raton, Fla., 1974).

Once the susceptibility of the substrate 104 material is determined, one can use the following equation: χ_{sub}+χ_{coat}=0, wherein χ_{sub} is the susceptibility of the substrate, and χ_{coat} is the susceptibility of the coating, when each of these is present in a 1/1 ratio. As will be apparent, the aforementioned equation is used when the coating and substrate are present in a 1/1 ratio. When other ratios are used other than a 1/1 ratio, the volume percent of each component (or its mass) must be taken into consideration in accordance with the equation: (volume percent of substrate x susceptibility of the substrate)+(volume percent of coating x susceptibility of the coating)=0. One may use a comparable formula in which the weight percent of each component is substituted for the volume percent, if the susceptibility is measured in terms of the weight percent.

By way of illustration, and in one embodiment, the uncoated substrate 104 may either comprise or consist essentially of niobium, which has a susceptibility of +195.0×10⁻⁶ centimeter-gram seconds at 298 degrees Kelvin.

In another embodiment, the substrate 104 may contain at least 98 molar percent of niobium and less than 2 molar percent of zirconium. Zirconium has a susceptibility of −122×0×10⁻⁶ centimeter-gram seconds at 293 degrees Kelvin. As will be apparent, because of the predominance of niobium, the net susceptibility of the uncoated substrate will be positive.

The substrate may comprise Nitinol. Nitinol is a paramagnetic alloy, an intermetallic compound of nickel and titanium; the alloy preferably contains from 50 to 60 percent of nickel, and it has a permeability value of about 1.002. The susceptibility of Nitinol is positive.

Nitinols with nickel content ranging from about 53 to 57 percent are known as “memory alloys” because of their ability to “remember” or return to a previous shape upon being heated which is an alloy of nickel and titanium, in an approximate 1/1 ratio. The susceptibility of Nitinol is positive.

The substrate 104 may comprise tantalum and/or titanium, each of which has a positive susceptibility. See, e.g., the CRC handbook cited above.

When the uncoated substrate has a positive susceptibility, the coating to be used for such a substrate should have a negative susceptibility. Referring again to said CRC handbook, it will be seen that the values of negative susceptibilities for various elements are −9.0 for beryllium, −280.1 for bismuth (s), −10.5 for bismuth (l), −6.7 for boron, −56.4 for bromine (l), −73.5 for bromine(g), −19.8 for cadmium(s), −18.0 for cadmium(l), −5.9 for carbon(dia), −6.0 for carbon (graph), −5.46 for copper(s), −6.16 for copper(l), −76.84 for germanium, −28.0 for gold(s), −34.0 for gold(l), −25.5 for indium, −88.7 for iodine(s), −23.0 for lead(s), −15.5 for lead(l), −19.5 for silver(s), −24.0 for silver(l), −15.5 for sulfur(alpha), −14.9 for sulfur(beta), −15.4 for sulfur(l), −39.5 for tellurium(s), −6.4 for tellurium(l), −37.0 for tin(gray), −31.7 for tin(gray), −4.5 for tin(l), −11.4 for zinc(s), −7.8 for zinc(l), and the like. As will be apparent, each of these values is expressed in units equal to the number in question×10⁻⁶ centimeter-gram seconds at a temperature at or about 293 degrees Kelvin. As will also be apparent, those materials which have a negative susceptibility value are often referred to as being diamagnetic.

By way of further reference, a listing of organic compounds that are diamagnetic is presented on pages E123 to E134 of the aforementioned “Handbook of Chemistry and Physics,” 63rd edition (CRC Press, Inc., Boca Raton, Fla., 1974).

In one embodiment, and referring again to the aforementioned “Handbook of Chemistry and Physics,” 63rd edition (CRC Press, Inc., Boca Raton, Fla., 1974), one or more of the following magnetic materials described below are preferably incorporated into the coating.

The desired magnetic materials, in this embodiment, preferably have a positive susceptibility, with values ranging from +1×10⁻⁶ centimeter-gram seconds at a temperature at or about 293 degrees Kelvin, to about 1×10⁷ centimeter-gram seconds at a temperature at or about 293 degrees Kelvin.

Thus, by way of illustration and not limitation, one may use materials such as Alnicol (see page E-112 of the CRC handbook), which is an alloy containing nickel, aluminum, and other elements such as, e.g., cobalt and/or iron. Thus, e.g., one my use silicon iron (see page E113 of the CRC handbook), which is an acid resistant iron containing a high percentage of silicon. Thus, e.g., one may use steel (see page 117 of the CRC handbook). Thus, e.g., one may use elements such as dyprosium, erbium, europium, gadolinium, hafnium, holmium, manganese, molybdenum, neodymium, nickel-cobalt, alloys of the above, and compounds of the above such as, e.g., their oxides, nitrides, carbonates, and the like.

Nullification of the Reactance of the Uncoated Substrate 104

In one preferred embodiment, and referring again to FIG. 4, the uncoated substrate 104 has an effective inductive reactance at a d.c. field of 1.5 Tesla that exceeds its capacitive reactance, whereas the coating 103 has a capacitive reactance that exceeds its inductive reactance. The coated (composite) substrate 100 has a net reactance that is preferably substantially zero.

As will be apparent, the effective inductive reactance of the uncoated stent 104 may be due to a multiplicity of factors including, e.g., the positive magnetic susceptibility of the materials which it is comprised of, the loop currents produced, the surface eddy produced, etc. Regardless of the source(s) of its effective inductive reactance, it can be “corrected” by the use of one or more coatings which provide, in combination, an effective capacitive reactance that is equal to the effective inductive reactance.

FIG. 4A is a sectional schematic illustration of a coated stent assembly 149, not drawn to scale, that illustrates a metallic stent 150 coated with a thin layer 152 of nanomagnetic material, a thin layer 154 of dielectric material, and thin layer 156 of conductive material, a thin layer 158 of dielectric material, and a thin layer 160 of conductive material.

Referring again to FIG. 4A, a conductive via 162 is shown extending from layer 160 to stent 150. As will be apparent, other via structures are possible. Thus, e.g., conductive struts 164/166 are contiguous with conductive layer 160.

As will be apparent to those skilled in the art, various combinations of vias, conductive materials, and dielectric materials may be used to create desired levels of capacitance and/or inductance, as well as resistance.

Thus, e.g. FIG. 4B illustrates capacitance in parallel that is created by dielectric material 158 sandwiched between parallel sets of conductive plates 160/160 and connected with leads 164/164 and 166/166. When 164 and 166 are connected, the capacitance is connected in parallel. As is known to those skilled in the art, the total parallel in capacitance is equal to the sum of the individual capacitances.

In one embodiment, shown in FIG. 4B, dielectric material 158 is broken into two segments by an insulating barrier 163. This insulating barrier may, e.g., have a relative dielectric constant of 1.

To form a parallel connection, the 166/166 pair may be connected to the 164/164 pair. The total capacitance then will be equal to the sum of the capacitances for this parallel connection.

Thus, e.g., FIG. 4C illustrates capacitance in series that is created between dielectric material 158 sandwiched between series conductive plates 160/160. A lead 164 is preferably connected between the conductive plates 160/160. As is known to those skilled in the art, the total capacitance in series is equal to 1 divided by 1/C₁+1/C₂.

Various other means of varying the inductive reactance, and the capacitive reactance, of the coated assembly by means of conductive vias, conductive layers, and dielectric layers, are known to those skilled in the art.

Creation of Vias in the Coated Substrate.

One may create vias, such as, e.g., via 162, by conventional means. Thus, e.g., one may create vias by the means disclosed in U.S. Pat. No. 3,988,823, the entire disclosure of which is hereby incorporated by reference into this specification. This patent claims “1. A method for fabricating a multilevel interconnected large scale integrated microelectronic circuit including vias therein having 0.5 mil and smaller openings for interlayer electrical communication of active devices and unit circuits on a silicon wafer in the microelectronic circuit, comprising the steps of: preparing a silicon wafer with active devices therein and interconnecting the active devices into functional unit circuits at a first level of aluminum metallization including means defining signal-connect pads terminating the unit circuits, by metal evaporation, masking and etching techniques; depositing a layer of pyrolytic silicon dioxide of approximate 0.5 micron thickness on the first level of metallization within a pyrolytic silicon dioxide deposition chamber for passivating the first level and for creating undesired openings in the pyrolytic layer; depositing a layer of photoresist material on the layer of pyrolytic silicon dioxide; placing on the photoresist layer a first mask defining positions of via openings to be etched in the layer of pyrolytic silicon dioxide and to be positioned over the signal-connect means; exposing the photoresist layer through the mask and thereafter removing the mask; developing, baking and further processing the exposed photoresist layer for forming therefrom an etch-resistant mask on the pyrolytic silicon dioxide layer with means defining openings in the etch-resistant mask positioned above the positions of the vias to be formed in the pyrolytic silicon dioxide layer; etching the pyrolytic silicon dioxide layer through the opening means in the etch-resistant mask by applying a mixture of acetic acid, ammonium fluoride and hydrogen fluoride over the etch-resistant mask for forming the vias having at most 0.5 mil openings; stripping the etch-resistant mask from and thereafter cleaning the etched pyrolytic silicon dioxide layer; forming aluminum-magnesium masks defining mushroom configurations, each comprising an aluminum crown and a magnesium stem on the etched pyrolytic silicon dioxide layer, with the stems covering the vias in the etched pyrolytic silicon dioxide layer; sputter depositing a layer of silicon dioxide of a thickness sufficient for adequate insulation over the pyrolytic silicon dioxide layer and over the mushroom-masks in a radio-frequency system for providing tapered deposits at the base of the stems and for closing any of the undesired openings in the pyrolytic silicon dioxide layer; removing the mushroom-masks by immersing the wafer in a dilute nitric acid bath for dissolving the magnesium stems of the mushroom-masks and thereby for floating-out the mushroom-masks for forming means in the RF-sputtered silicon dioxide layer defining openings of at least 3 mil diameters over the vias having at most the 0.5 mil openings in the pyrolytic silicon dioxide layer; forming a second level of aluminum metallization defining interconnections among the active devices and the unit circuits over the RF-sputtered silicon dioxide layer and the pyrolytic silicon dioxide layer exposed and surrounded by the opening means for making low resistance electrical contact through the vias and for effecting continuity of the second level of aluminum through the opening means and the vias; further processing of the silicon wafer from the second level of metallization into the integrated microelectronic circuit; and annealing of the circuit at approximately 400° C. for approximately 16 hours for reducing any contact resistance through the opening means and the vias to a uniform, acceptable level.”

By way of further illustration, and referring to U.S. Pat. No. 4,753,709, the entire disclosure of which is hereby incorporated by reference into this disclosure, one may form vias by the etching process of claim 1 of this patent, which describes “1. A method for fabricating an integrated circuit on a semiconductor chip, comprising: forming a conductive interconnection layer comprised of silicon; forming a silicide film on the surface of said conductive layer; depositing a dielectric film covering said conductive layer; etching said dielectric film so that selected locations of said silicide film on said conductive layer are exposed; and depositing a metal interconnection layer.”

By way of yet further illustration, and referring to U.S. Pat. No. 6,784,096, the entire disclosure of which is hereby incorporated by reference into this specification, one may form barrier layers in high aspect vias by a process comprising the steps of “A method of forming a barrier layer comprising: (a) providing a substrate having: a metal feature; a dielectric layer formed over the metal feature; and a via having sidewalls and a bottom, the via extending through the dielectric layer to expose the metal feature; (b) forming a barrier layer over the sidewalls and bottom of the via using atomic layer deposition, the barrier layer having sufficient thickness to servo as a diffusion barrier to at least one of atoms of the metal feature and atoms of a used layer formed over the barrier layer; (c) removing at least a portion of the barrier layer from the bottom of the via by sputter etching the substrate within a high density plasma physical vapor deposition (HDPPVD) chamber having a plasma ion density of at least 1010 ions/cm3 and configured for seed layer deposition, wherein a bias is applied to the substrate during at least a portion of the sputter etching; and (d) depositing a seed layer on the sidewalls and bottom of the via within the HDPPVD chamber.”

The aforementioned patents are merely illustrative of many United States patents that describe via forming processes. Thus, e.g., by way of yet further illustration, one may use the via forming processes described in U.S. Pat. No. 4,258,468 (forming vias through multilayer circuit boards), U.S. Pat. No. 4,670,091 (forming vias on integrated circuits), U.S. Pat. No. 4,780,770 (planarized process for forming vias), U.S. Pat. No. 5,091,339 (trenching techniques for forming vias and channels), U.S. Pat. No. 5,108,562 (electrolytic method for forming vias), U.S. Pat. No. 5,293,025 (method for forming vias in multilayer circuits), U.S. Pat. No. 5,424,245 (forming vias through two-sided substrate), U.S. Pat. No. 5,510,294 (forming vias for multilevel metallization), U.S. Pat. No. 5,593,606 (ultraviolet laser system and method for forming vias in multi-layered targets), U.S. Pat. No. 5,593,921 (method of forming vias), U.S. Pat. No. 5,683,758 (method of forming vias), U.S. Pat. Nos. 5,825,076, 5,861,673 (method for forming vias in multi-level integrated circuits), U.S. Pat. No. 5,874,369 (method for forming vias in a dielectric film), U.S. Pat. No. 5,904,566 (reactive ion etch method for forming vias), U.S. Pat. No. 6,037,262 (process for forming vias and trenches for metal lines in multiple dielectric layers), U.S. Pat. No. 6,096,655 (method for forming vias in an insulation layer for a dual-damascene multilevel interconnection structure), U.S. Pat. No. 6,140,221 (method for forming vias through porous dielectric materials), U.S. Pat. No. 6,180,518 (method of forming vias in a low dielectric constant material), U.S. Pat. No. 6,429,049 (laser method for forming vias), U.S. Pat. No. 6,433,301 (beam shaping and projection imaging with solid state UV Gaussian beam to form vias), U.S. Pat. No. 6,475,889 (method of forming vias in silicon carbide), U.S. Pat. No. 6,518,171 (dual damascene process), U.S. Pat. Nos. 6,649,497, 6,791,060, and the like. The entire disclosure of each of these United States patents is hereby incorporated by reference into this specification.

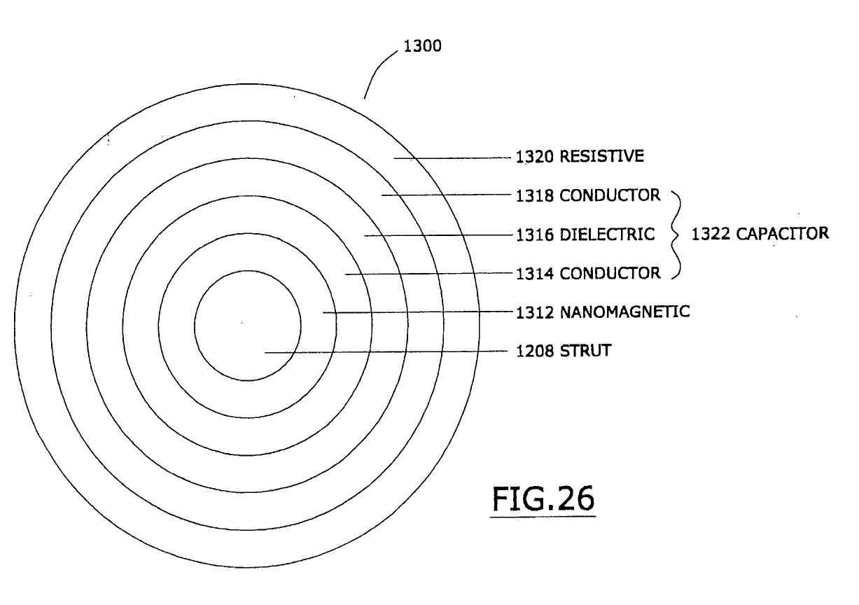

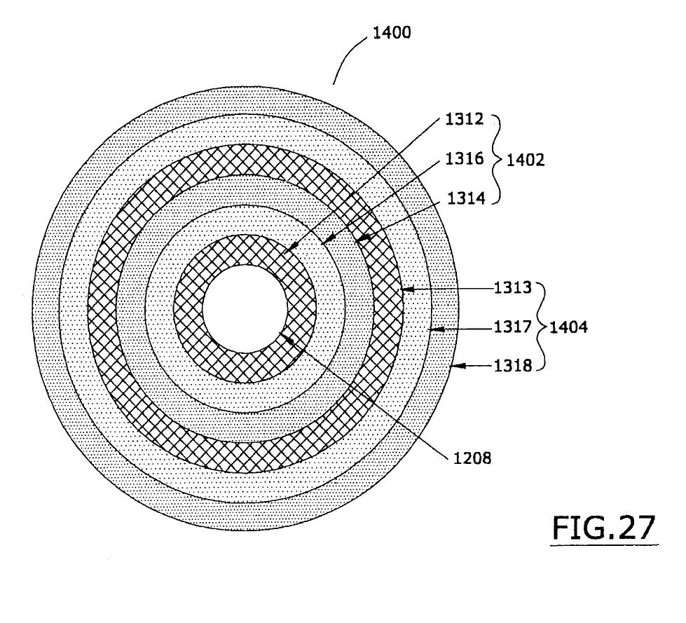

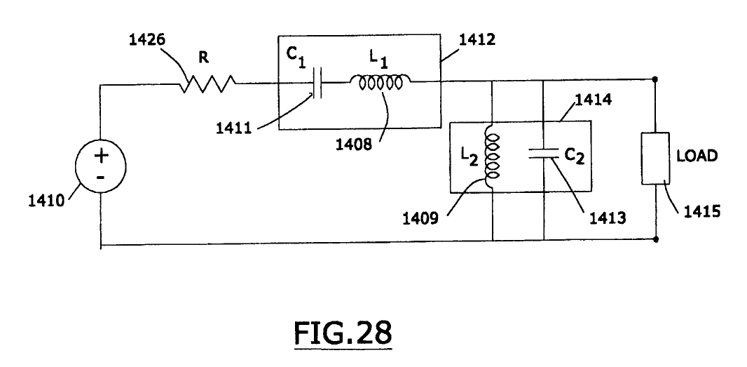

FIG. 4D is a schematic of an arrangement 170 comprised of three coated inductors 172, 174, and 176. In the embodiment depicted, the three coated inductors 172, 174, and 176 may comprise, e.g., portions of nanomagnetic coatings disposed around a conductor (see, e.g., FIGS. 26 and 27).

Referring to FIG. 4D, the equivalent inductors 172/174/176 are interconnected by means of conductive vias 178 and 180 to form a series connection. As is well known to those skilled in the art, in series the inductances add, the total being the sum of each individual inductance.

FIG. 4E, by comparison, illustrates equivalent inductors 172/174/176 being connected in parallel by conductive vias 178 and 180. As is known, the total inductance for this arrangement defined by the formula 1/(1/L₁+1/L₂+1/L₃).

As will be apparent to those skilled in the art, comparable means of varying the capacitance are readily available.

Imaging of Restenosis

Referring again to FIG. 4, and in the embodiment depicted, plaque particles 130,132 are disposed on the inside of substrate 104. When the net reactance of the coated substrate 104 is essentially zero, the imaging field 140 can pass substantially unimpeded through the coating 103 and the substrate 104 and interact with the plaque particles 130/132 to produce imaging signals 141.

The imaging signals 141 are able to pass back through the substrate 104 and the coating 103 because the net reactance is substantially zero. Thus, these imaging signals are able to be received and processed by the MRI apparatus.

Thus, by the use of applicants' technology, one may negate the negative substrate effect and, additionally, provide pathways for the image signals to interact with the desired object to be imaged (such as, e.g., the plaque particles) and to produce imaging signals that are capable of escaping the substrate assembly and being received by the MRI apparatus.

Referring again to FIG. 4, and in one preferred embodiment, when an MRI field is present, the entire assembly 13, including the biological material 130/132, preferably presents a direct current magnetic susceptibility that is plus or minus 1×10⁻³ centimeter-gram-seconds (cgs) and, more preferably, plus or minus 1×10⁻⁴ centimeter-gram-seconds. In one embodiment, the d.c. susceptibility of the assembly 13 is equal to plus or minus 1×10⁻⁵ centimeter-gram-seconds. In another embodiment, the d.c. susceptibility of the assembly 13 is equal to plus or minus 10×−6 centimeter-gram-seconds.

Referring again to FIG. 4, each of the components of assembly 13 has its own value of magnetic susceptibility. Thus, the biological material 130/132 has a magnetic susceptibility of S₁. Thus, the substrate 104 has a magnetic susceptibility of S_{2.} Thus, the coating 103 has a magnetic susceptibility of S₃.

Each of the components of the assembly 13 makes a contribution to the total magnetic susceptibility of such assembly, depending upon (a) whether its magnetic susceptibility is positive or negative, (b) the amount of its positive or negative susceptibility value, and (c) the percentage of the total mass that the individual component represents.

In determining the total susceptibility of the assembly 13, one can first determine the product of Mc and Sc, wherein Mc is the weight fraction of that component (the weight of that component divided by the total weight of all components in the assembly 6000).

In one preferred process, the McSc values for the nanomagnetic material 120 are chosen to, when appropriate, correct for the total McSc values of all of the other components (including the biological material 130/132) such that, after such correction(s), the total susceptibility of the assembly 13 is plus or minus 1×10⁻³ centimeter-gram-seconds (cgs) and, more preferably, plus or minus 1×10⁻⁴ centimeter-gram-seconds. In one embodiment, the d.c. susceptibility of the assembly 13 is equal to plus or minus 1×10⁻⁵ centimeter-gram-seconds. In another embodiment, the d.c. susceptibility of the assembly 13 is equal to plus or minus 1×10⁻⁶ centimeter-gram-seconds.

As will be apparent, there may be other materials/components in the assembly 13 whose values of positive or negative susceptibility, and/or their mass, may be chosen such that the total magnetic susceptibility of the assembly is plus or minus 1×10⁻³ centimeter-gram-seconds (cgs) and, more preferably, plus or minus 1×10⁻⁴ centimeter-gram-seconds. Similarly, the configuration of the substrate may be varied in order to vary its magnetic susceptibility properties and/or other properties.

Cancellation of the Positive Susceptibility of a Nitinol Stent

In one preferred embodiment, illustrated in FIG. 5, a stent 200 constructed from Nitinol is comprised of struts 202, 204, 206, and 208 coated with a layer of elemental bismuth. As is known to those skilled in the art, Nitinol is a paramagnetic alloy that was developed by the Naval Ordnance Laboratory; it is an intermetallic compound of nickel and titanium. See, e.g., page 552 of George S. Brady et al.'s “Materials Handbook,” Thirteenth Edition (McGraw-Hill Company, New York, N.Y., 1991).

Referring again to FIG. 5, and to the preferred embodiment depicted therein, the stent 200 is preferably cylindrical with a diameter (not shown) of less than 1 centimeter and a length 210 of about 3 centimeters. Each strut, such as strut 202, is preferably arcuate, having an effective diameter 212 of less than about 1 millimeter.

As is known to those skilled in the art, the magnetic permeability of the Nitinol material is about 1.003; and its susceptibility is about 0.03 centimeter-grams-seconds (cgs). In order to nullify the susceptibility, one can introduce a diamagnetic material, such as bismuth, that has a negative susceptibility. In one embodiment, a bismuth coating with a thickness of form about 10 to about 20 microns is deposited upon each of the struts 202.

Thus, and as will be apparent from the discussions presented in other parts of this specification, the susceptibility for these struts 202 becomes substantially zero, whereby there is no substantial direct current (d.c.) susceptibility distortion in the MRI field. As used herein, the term “substantially zero” refers to a net susceptibility of from about 0.9 to about 1.1.

As will be apparent, when applicant's nanomagnetic coating 103 is added to such stent 210, the amount and type of the coating is chosen such that the net susceptibility for the struts is still preferably substantially zero,

As will be also be apparent, susceptibility varies with both direct current and alternating current. It is desired that, with the composite coating 103 described hereinabove, the susceptibility at a direct current field of about 1.5 Tesla (which is also the slope of the plot of magnetization versus the applied magnetic field), should preferably be from about 0.9 to about 1.1.

Incorporation by Reference of U.S. Pat. No. 6,713,671

U.S. patent application Ser. No. 10/303,264 (and also U.S. Pat. No. 6,713,671) discloses a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters; the nanomagnetic material comprises nanomagnetic particles, and these nanomagnetic particles respond to an externally applied magnetic field by realigning to the externally applied field. Such a shielded assembly and/or the substrate thereof and/or the shield thereof may be used in the processes, compositions, and/or constructs of this invention.

As is disclosed in U.S. Pat. No. 6,713,617, the entire disclosure of which is hereby incorporated by reference into this specification, in one embodiment the substrate used may be, e.g., comprised of one or more conductive material(s) that have a resistivity at 20 degrees Centigrade of from about 1 to about 100 microohm-centimeters. Thus, e.g., the conductive material(s) may be silver, copper, aluminum, alloys thereof, mixtures thereof, and the like.

In one embodiment, the substrate consists consist essentially of such conductive material. Thus, e.g., it is preferred not to use, e.g., copper wire coated with enamel in this embodiment.

In the first step of the process preferably used to make this embodiment of the invention, (see step 40 of FIG. 1 of U.S. Pat. No. 6,713,671), conductive wires are coated with electrically insulative material. Suitable insulative materials include nano-sized silicon dioxide, aluminum oxide, cerium oxide, yttrium-stabilized zirconia, silicon carbide, silicon nitride, aluminum nitride, and the like. In general, these nano-sized particles will have a particle size distribution such that at least about 90 weight percent of the particles have a maximum dimension in the range of from about 10 to about 100 nanometers.

In such process, the coated conductors may be prepared by conventional means such as, e.g., the process described in U.S. Pat. No. 5,540,959, the entire disclosure of which is hereby incorporated by reference into this specification. Alternatively, one may coat the conductors by means of the processes disclosed in a text by D. Satas on “Coatings Technology Handbook” (Marcel Dekker, Inc., New York, N.Y., 1991). As is disclosed in such text, one may use cathodic arc plasma deposition (see pages 229 et seq.), chemical vapor deposition (see pages 257 et seq.), sol-gel coatings (see pages 655 et seq.), and the like.

FIG. 2 of U.S. Pat. No. 6,713,671 is a sectional view of the coated conductors 14/16. In the embodiment depicted in such FIG. 2, it will be seen that conductors 14 and 16 are separated by insulating material 42. In order to obtain the structure depicted in such FIG. 2, one may simultaneously coat conductors 14 and 16 with the insulating material so that such insulators both coat the conductors 14 and 16 and fill in the distance between them with insulation.

Referring again to such FIG. 2 of U.S. Pat. No. 6,713,671, the insulating material 42 that is disposed between conductors 14/16, may be the same as the insulating material 44/46 that is disposed above conductor 14 and below conductor 16. Alternatively, and as dictated by the choice of processing steps and materials, the insulating material 42 may be different from the insulating material 44 and/or the insulating material 46. Thus, step 48 of the process of such FIG. 2 describes disposing insulating material between the coated conductors 14 and 16. This step may be done simultaneously with step 40; and it may be done thereafter.

Referring again to such FIG. 2, and to the preferred embodiment depicted therein, the insulating material 42, the insulating material 44, and the insulating material 46 each generally has a resistivity of from about 1,000,000,000 to about 10,000,000,000,000 ohm-centimeters.

Referring again to FIG. 2 of U.S. Pat. No. 6,713,671, after the insulating material 42/44/46 has been deposited, and in one embodiment, the coated conductor assembly is preferably heat treated in step 50. This heat treatment often is used in conjunction with coating processes in which the heat is required to bond the insulative material to the conductors 14/16.

The heat-treatment step may be conducted after the deposition of the insulating material 42/44/46, or it may be conducted simultaneously therewith. In either event, and when it is used, it is preferred to heat the coated conductors 14/16 to a temperature of from about 200 to about 600 degrees Centigrade for from about 1 minute to about 10 minutes.

Referring again to FIG. 1A of U.S. Pat. No. 6,713,671 and in step 52 of the process, after the coated conductors 14/16 have been subjected to heat treatment step 50, they are allowed to cool to a temperature of from about 30 to about 100 degrees Centigrade over a period of time of from about 3 to about 15 minutes.

One need not invariably heat treat and/or cool. Thus, referring to such FIG. 1A, one may immediately coat nanomagnetic particles onto to the coated conductors 14/16 in step 54 either after step 48 and/or after step 50 and/or after step 52.

Referring again to FIG. 1A of U.S. Pat. No. 6,713,671 in step 54, nanomagnetic materials are coated onto the previously coated conductors 14 and 16. This is best shown in FIG. 2 of such patent, wherein the nanomagnetic particles are identified as particles 24.

In general, and as is known to those skilled in the art, nanomagnetic material is magnetic material which has an average particle size less than 100 nanometers and, preferably, in the range of from about 2 to 50 nanometers. Reference may be had, e.g., to U.S. Pat. No. 5,889,091 (rotationally free nanomagnetic material), U.S. Pat. Nos. 5,714,136, 5,667,924, and the like. The entire disclosure of each of these United States patents is hereby incorporated by reference into this specification.

In general, the thickness of the layer of nanomagnetic material deposited onto the coated conductors 14/16 is less than about 5 microns and generally from about 0.1 to about 3 microns.

Referring again to FIG. 2 of U.S. Pat. No. 6,713,671, after the nanomagnetic material is coated in step 54, the coated assembly may be optionally heat-treated in step 56. In this optional step 56, it is preferred to subject the coated conductors 14/16 to a temperature of from about 200 to about 600 degrees Centigrade for from about 1 to about 10 minutes.

In one embodiment, illustrated in FIG. 3 of U.S. Pat. No. 6,713,671, one or more additional insulating layers 43 are coated onto the assembly depicted in FIG. 2 of such patent. This is conducted in optional step 58 (see FIG. 1A of such patent).

FIG. 4 of U.S. Pat. No. 6,713,671 is a partial schematic view of the assembly 11 of FIG. 2 of such patent, illustrating the current flow in such assembly. Referring again to FIG. 4 of U.S. Pat. No. 6,713,671, it will be seen that current flows into conductor 14 in the direction of arrow 60, and it flows out of conductor 16 in the direction of arrow 62. The net current flow through the assembly 11 is zero; and the net Lorentz force in the assembly 11 is thus zero. Consequently, even high current flows in the assembly 11 do not cause such assembly to move.

Referring again to FIG. 4 of U.S. Pat. No. 6,713,671 conductors 14 and 16 are substantially parallel to each other. As will be apparent, without such parallel orientation, there may be some net current and some net Lorentz effect.

In the embodiment depicted in such FIG. 4, and in one preferred aspect thereof, the conductors 14 and 16 preferably have the same diameters and/or the same compositions and/or the same length.

Referring again to FIG. 4 of U.S. Pat. No. 6,713,671, the nanomagnetic particles 24 are present in a density sufficient so as to provide shielding from magnetic flux lines 64. Without wishing to be bound to any particular theory, applicant believes that the nanomagnetic particles 24 trap and pin the magnetic lines of flux 64.

In order to function optimally, the nanomagnetic particles 24 preferably have a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Referring again to FIG. 4 of U.S. Pat. No. 6,713,671, the entire disclosure of which is hereby incorporated by reference into this specification, the layer of nanomagnetic particles 24 preferably has a saturation magnetization, at 25 degrees Centigrade, of from about 1 to about 36,000 Gauss, or higher. In one embodiment, the saturation magnetization at room temperature of the nanomagnetic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification.

In one embodiment, it is preferred to utilize a thin film with a thickness of less than about 2 microns and a saturation magnetization in excess of 20,000 Gauss. The thickness of the layer of nanomagnetic material is measured from the bottom surface of the layer that contains such material to the top surface of such layer that contains such material; and such bottom surface and/or such top surface may be contiguous with other layers of material (such as insulating material) that do not contain nanomagnetic particles.

Thus, e.g., one may make a thin film in accordance with the procedure described at page 156 of Nature, Volume 407, Sep. 14, 2000, that describes a multilayer thin film has a saturation magnetization of 24,000 Gauss.

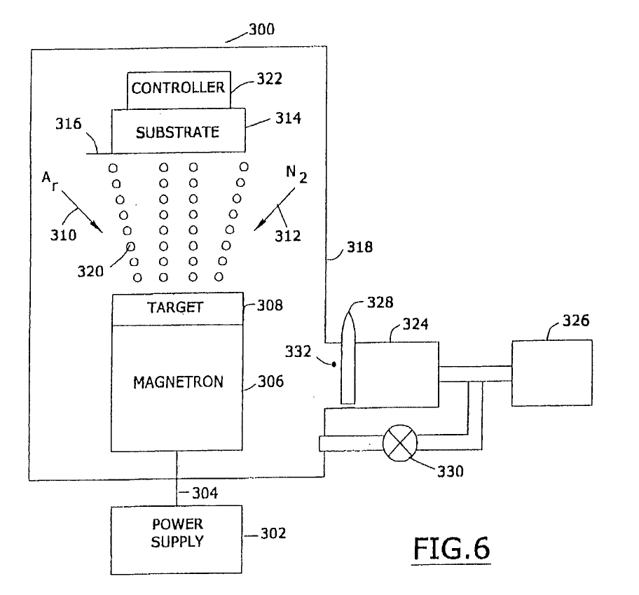

Referring again to FIG. 6 of U.S. Pat. No. 6,713,671, the film 104 is adapted to reduce the magnetic field strength at point 108 (which is disposed less than 1 centimeter above film 104) by at least about 50 percent. Thus, if one were to measure the magnetic field strength at point 108, and thereafter measure the magnetic field strength at point 110 (which is disposed less than 1 centimeter below film 104), the latter magnetic field strength would be no more than about 50 percent of the former magnetic field strength. Put another way, the film 104 has a magnetic shielding factor of at least about 0.5.

Referring again to FIG. 6 of U.S. Pat. No. 6,713,671, in one embodiment, the film 104 has a magnetic shielding factor of at least about 0.9, i.e., the magnetic field strength at point 110 is no greater than about 10 percent of the magnetic field strength at point 108. Thus, e.g., the static magnetic field strength at point 108 can be, e.g., one Tesla, whereas the static magnetic field strength at point 110 can be, e.g., 0.1 Tesla. Furthermore, the time-varying magnetic field strength of a 100 milliTesla would be reduced to about 10 milliTesla of the time-varying field.

An MRI Imaging Process

In one embodiment of the invention, best illustrated in FIG. 4, a coated stent 100 is imaged by a MRI imaging process. As will be apparent to those skilled in the art, the process depicted in FIG. 4 can be used with reference to other medical devices such as, e.g., a coated brachytherapy seed.

In the first step of this process, the coated stent 100 is contacted with the radio-frequency, direct current, and gradient fields normally associated with MRI imaging processes; these fields are discussed elsewhere in this specification. They are depicted as a MRI imaging signal 140 in FIG. 4.

In the second step of this process, the MRI imaging signal 140 penetrates the coated stent 100 and interacts with material disposed on the inside of such stent, such as, e.g., plaque particles 130 and 132. This interaction produces a signal best depicted as arrow 141 in FIG. 4.

In one embodiment, the signal 440 is substantially unaffected by its passage through the coated stent 100. Thus, in this embodiment, the radio-frequency field that is disposed on the outside of the coated stent 100 is substantially the same as the radio-frequency field that passes through and is disposed on the inside of the coated stent 100.

By comparison, when the stent (not shown) is not coated with the coatings of this invention, the characteristics of the signal 140 are substantially varied by its passage through the uncoated stent. Thus, with such uncoated stent, the radio-frequency signal that is disposed on the outside of the stent (not shown) differs substantially from the radio-frequency field inside of the uncoated stent (not shown). In some cases, because of substrate effects, substantially none of such radio-frequency signal passes through the uncoated stent (not shown).

In the third step of this process, and in one embodiment thereof, the MRI field(s) interact with material disposed on the inside of coated stent 100 such as, e.g., plaque particles 130 and 132. This interaction produces a signal 141 by means well known to those in the MRI imaging art.

In the fourth step of the preferred process of this invention, the signal 141 passes back through the coated stent 100 in a manner such that it is substantially unaffected by the coated stent 100. Thus, in this embodiment, the radio-frequency field that is disposed on the inside of the coated stent 100 is substantially the same as the radio-frequency field that passes through and is disposed on the outside of the coated stent 100.

By comparison, when the stent (not shown) is not coated with the coatings of this invention, the characteristics of the signal 141 are substantially varied by its passage through the uncoated stent. Thus, with such uncoated stent, the radio-frequency signal that is disposed on the inside of the stent (not shown) differs substantially from the radio-frequency field outside of the uncoated stent (not shown). In some cases, because of substrate effects, substantially none of such signal 141 passes through the uncoated stent (not shown).

A Process for Preparation of an Iron-Containing Thin Film

In one preferred embodiment of the invention, a sputtering technique is used to prepare an AlFe thin film or particles, as well as comparable thin films containing other atomic moieties, or particles, such as, e.g., elemental nitrogen, and elemental oxygen. Conventional sputtering techniques may be used to prepare such films by sputtering. See, for example, R. Herrmann and G. Brauer, “D.C.- and R.F. Magnetron Sputtering,” in the “Handbook of Optical Properties: Volume 1—Thin Films for Optical Coatings,” edited by R. E. Hummel and K. H. Guenther (CRC Press, Boca Raton, Fla., 1955). Reference also may be had, e.g., to M. Allendorf, “Report of Coatings on Glass Technology Roadmap Workshop,” Jan. 18-19, 2000, Livermore, Calif.; and also to U.S. Pat. No. 6,342,134, “Method for producing piezoelectric films with rotating magnetron sputtering system.” The entire disclosure of each of these prior art documents is hereby incorporated by reference into this specification.

One may utilize conventional sputtering devices in this process. By way of illustration and not limitation, a typical sputtering system is described in U.S. Pat. No. 5,178,739, the entire disclosure of which is hereby incorporated by reference into this specification. As is disclosed in this patent, “ . . . a sputter system 10 includes a vacuum chamber 20, which contains a circular end sputter target 12, a hollow, cylindrical, thin, cathode magnetron target 14, a RF coil 16 and a chuck 18, which holds a semiconductor substrate 19. The atmosphere inside the vacuum chamber 20 is controlled through channel 22 by a pump (not shown). The vacuum chamber 20 is cylindrical and has a series of permanent, magnets 24 positioned around the chamber and in close proximity therewith to create a multiple field configuration near the interior surface 15 of target 12. Magnets 26, 28 are placed above end sputter target 12 to also create a multipole field in proximity to target 12. A singular magnet 26 is placed above the center of target 12 with a plurality of other magnets 28 disposed in a circular formation around magnet 26. For convenience, only two magnets 24 and 28 are shown. The configuration of target 12 with magnets 26, 28 comprises a magnetron sputter source 29 known in the prior art, such as the Torus-10E system manufactured by K. Lesker, Inc. A sputter power supply 30 (DC or RF) is connected by a line 32 to the sputter target 12. A RF supply 34 provides power to RF coil 16 by a line 36 and through a matching network 37. Variable impedance 38 is connected in series with the cold end 17 of coil 16. A second sputter power supply 39 is connected by a line 40 to cylindrical sputter target 14. A bias power supply 42 (DC or RF) is connected by a line 44 to chuck 18 in order to provide electrical bias to substrate 19 placed thereon, in a manner well known in the prior art.”

By way of yet further illustration, other conventional sputtering systems and processes are described in U.S. Pat. No. 5,569,506 (a modified Kurt Lesker sputtering system), U.S. Pat. No. 5,824,761 (a Lesker Torus 10 sputter cathode), U.S. Pat. Nos. 5,768,123, 5,645,910, 6,046,398 (sputter deposition with a Kurt J. Lesker Co. Torus 2 sputter gun), U.S. Pat. Nos. 5,736,488, 5,567,673, 6,454,910, and the like. The entire disclosure of each of these United States patents is hereby incorporated by reference into this specification.

By way of yet further illustration, one may use the techniques described in a paper by Xingwu Wang et al. entitled “Technique Devised for Sputtering AIN Thin Films,” published in “the Glass Researcher,” Volume 11, No. 2 (Dec. 12, 2002).

In one preferred embodiment, a magnetron sputtering technique is utilized, with a Lesker Super System 111 system. The vacuum chamber of this system is preferably cylindrical, with a diameter of approximately one meter and a height of approximately 0.6 meters. The base pressure used is from about 0.001 to 0.0001 Pascals. In one aspect of this process, the target is a metallic FeAl disk, with a diameter of approximately 0.1 meter. The molar ratio between iron and aluminum used in this aspect is approximately 70/30. Thus, the starting composition in this aspect is almost non-magnetic. See, e.g., page 83 (FIG. 3.1aii) of R. S. Tebble et al.'s “Magnetic Materials” (Wiley-Interscience, New York, N.Y., 1969); this Figure discloses that a bulk composition containing iron and aluminum with at least 30 mole percent of aluminum (by total moles of iron and aluminum) is substantially non-magnetic.

In this aspect, to fabricate FeAl films, a DC power source is utilized, with a power level of from about 150 to about 550 watts (Advanced Energy Company of Colorado, model MDX Magnetron Drive). The sputtering gas used in this aspect is argon, with a flow rate of from about 0.0012 to about 0.0018 standard cubic meters per second. To fabricate FeAlN films in this aspect, in addition to the DC source, a pulse-forming device is utilized, with a frequency of from about 50 to about 250 MHz (Advanced Energy Company, model Sparc-le V). One may fabricate FeAlO films in a similar manner but using oxygen rather than nitrogen.

In this aspect, a typical argon flow rate is from about (0.9 to about 1.5)×10⁻³ standard cubic meters per second; a typical nitrogen flow rate is from about (0.9 to about 1.8)×10⁻³ standard cubic meters per second; and a typical oxygen flow rate is from about. (0.5 to about 2)×10⁻³ standard cubic meters per second. During fabrication, the pressure typically is maintained at from about 0.2 to about 0.4 Pascals. Such a pressure range has been found to be suitable for nanomagnetic materials fabrications. In one embodiment, it is preferred that both gaseous nitrogen and gaseous oxygen are present during the sputtering process.

In this aspect, the substrate used may be either flat or curved. A typical flat substrate is a silicon wafer with or without a thermally grown silicon dioxide layer, and its diameter is preferably from about 0.1 to about 0.15 meters. A typical curved substrate is an aluminum rod or a stainless steel wire, with a length of from about 0.10 to about 0.56 meters and a diameter of from (about 0.8 to about 3.0)×10⁻³ meters. The distance between the substrate and the target is preferably from about 0.05 to about 0.26 meters.

In this aspect, in order to deposit a film on a wafer, the wafer is fixed on a substrate holder. The substrate may or may not be rotated during deposition. In one embodiment, to deposit a film on a rod or wire, the rod or wire is rotated at a rotational speed of from about 0.01 to about 0.1 revolutions per second, and it is moved slowly back and forth along its symmetrical axis with a maximum speed of about 0.01 meters per second.

In this aspect, to achieve a film deposition rate on the flat wafer of 5×10⁻¹⁰ meters per second, the power required for the FeAl film is 200 watts, and the power required for the FeAlN film is 500 watts. The resistivity of the FeAlN film is approximately one order of magnitude larger than that of the metallic FeAl film. Similarly, the resistivity of the FeAlO film is about one order of magnitude larger than that of the metallic FeAl film.

Iron containing magnetic materials, such as FeAl, FeAlN and FeAlO, FeAlNO, FeCoAlNO, and the like, may be fabricated by sputtering. The magnetic properties of those materials vary with stoichiometric ratios, particle sizes, and fabrication conditions; see, e.g., R. S. Tebble and D. J. Craik, “Magnetic Materials”, pp. 81-88, Wiley-Interscience, New York, 1969 As is disclosed in this reference, when the iron molar ratio in bulk FeAl materials is less than 70 percent or so, the materials will no longer exhibit magnetic properties.

However, it has been discovered that, in contrast to bulk materials, a thin film material often exhibits different properties.

A Preferred Sputtering Process

On Dec. 29, 2003, applicants filed U.S. patent application Ser. No. 10/747,472, for “Nanoelectrical Compositions.” The entire disclosure of this United States patent application is hereby incorporated by reference into this specification.

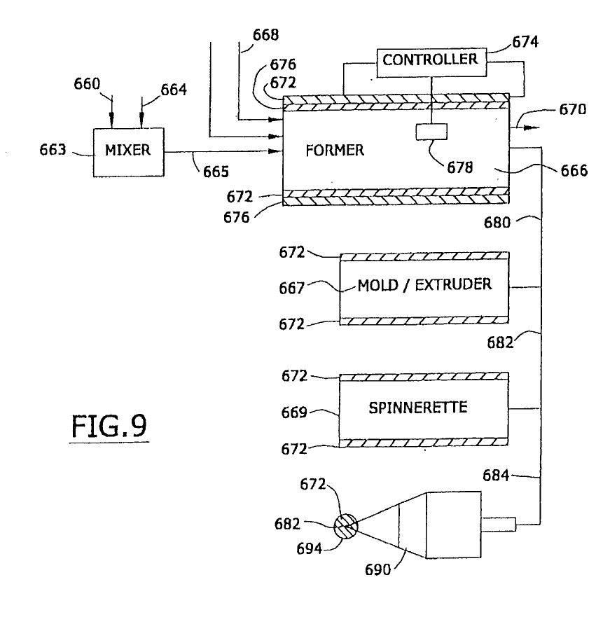

U.S. Ser. No. 10/747,472, at pages 10-15 thereof (and by reference to its FIG. 9), described the “ . . . preparation of a doped aluminum nitride assembly.” This portion of U.S. Ser. No. 10/747,472 is specifically incorporated by reference into this specification. It is also described below, by reference to FIG. 6, which is similar to the FIG. 9 of U.S. Ser. No. 10/747,472 but utilizes different reference numerals.

The system depicted in FIG. 6 may be used to prepare an assembly comprised of moieties A, B, and C that are described elsewhere in this specification. FIG. 5 will be described hereinafter with reference to one of the preferred ABC moieties, i.e., aluminum nitride doped with magnesium.

FIG. 6 is a schematic of a deposition system 300 comprised of a power supply 302 operatively connected via line 304 to a magnetron 306. Disposed on top of magnetron 306 is a target 308. The target 308 is contacted by gas 310 and gas 312, which cause sputtering of the target 308. The material so sputtered contacts substrate 314 when allowed to do so by the absence of shutter 316.

In one preferred embodiment, the target 308 is mixture of aluminum and magnesium atoms in a molar ratio of from about 0.05 to about 0.5 Mg/(Al+Mg). In one aspect of this embodiment, the ratio of Mg/(Al+Mg) is from about 0.08 to about 0.12. These targets are commercially available and are custom made by companies such as, e.g., Kurt Lasker and Company of Pittsburgh, Pa.

The power supply 302 preferably provides pulsed direct current. Generally, power supply 302 provides power in excess of 300 watts, preferably in excess of 500 watts, and more preferably in excess of 1,000 watts. In one embodiment, the power supplied by power supply 302 is from about 1800 to about 2500 watts.

The power supply preferably provides rectangular-shaped pulses with a duration (pulse width) of from about 10 nanoseconds to about 100 nanoseconds. In one embodiment, the pulse width is from about 20 to about 40 nanoseconds.

In between adjacent pulses, preferably substantially no power is delivered. The time between adjacent pulses is generally from about 1 microsecond to about 10 microseconds and is generally at least 100 times greater than the pulse width. In one embodiment, the repetition rate of the rectangular pulses is preferably about 150 kilohertz.

One may use a conventional pulsed direct current (d.c.) power supply. Thus, e.g., one may purchase such a power supply from Advanced Energy Company of Colorado, and/or from ENI Company of Rochester, N.Y.

The pulsed d.c. power from power supply 302 is delivered to a magnetron 306 that creates an electromagnetic field near target 308. In one embodiment, a magnetic field has a magnetic flux density of from about 0.01 Tesla to about 0.1 Tesla.

As will be apparent, because the energy provided to magnetron 306 preferably comprises intermittent pulses, the resulting magnetic fields produced by magnetron 306 will also be intermittent. Without wishing to be bound to any particular theory, applicants believe that the use of such intermittent electromagnetic energy yields better results than those produced by continuous radio-frequency energy.

Referring again to FIG. 6, it will be seen that the process depicted therein preferably is conducted within a vacuum chamber 318 in which the base pressure is from about 1×10⁻⁸ Torr to about 0.000005 Torr. In one embodiment, the base pressure is from about 0.000001 to about 0.000003 Torr.

The temperature in the vacuum chamber 318 generally is ambient temperature prior to the time sputtering occurs.

In one aspect of the embodiment illustrated in FIG. 6, argon gas is fed via line 310, and nitrogen gas is fed via line 312 so that both impact target 308, preferably in an ionized state. In another embodiment of the invention, argon gas, nitrogen gas, and oxygen gas are fed via target 312.

The argon gas, and the nitrogen gas, are fed at flow rates such that the flow rate of the argon gas divided by the flow rate of the nitrogen gas preferably is from about 0.6 to about 1.2. In one aspect of this embodiment, such ratio of argon to nitrogen is from about 0.8 to about 0.95. Thus, for example, the flow rate of the argon may be 20 standard cubic centimeters per minute, and the flow rate of the nitrogen may be 23 standard cubic feet per minute.

The argon gas, and the nitrogen gas, contact a target 308 that is preferably immersed in an electromagnetic field. This field tends to ionize the argon and the nitrogen, providing ionized species of both gases. It is such ionized species that bombard target 308.

In one embodiment, target 308 may be, e.g., pure aluminum. In one preferred embodiment, however, target 308 is aluminum doped with minor amounts of one or more of the aforementioned moieties B.

In the latter embodiment, the moieties B are preferably present in a concentration of from about 1 to about 40 molar percent, by total moles of aluminum and moieties B. It is preferred to use from about 5 to about 30 molar percent of such moieties B.

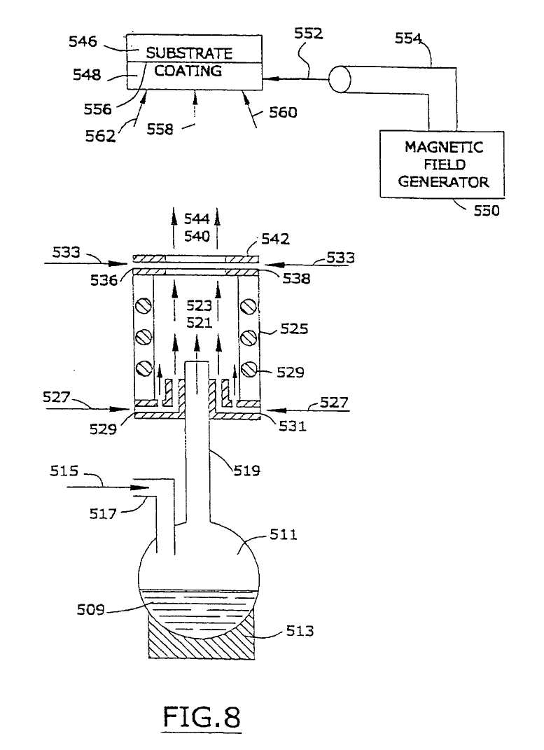

The ionized argon gas, and the ionized nitrogen gas, after impacting the target 308, creates a multiplicity of sputtered particles 320. In the embodiment illustrated in FIG. 8 the shutter 316 prevents the sputtered particles from contacting substrate 314.

When the shutter 316 is removed, however, the sputtered particles 320 can contact and coat the substrate 314.

In one embodiment, illustrated in FIG. 6 the temperature of substrate 314 is controlled by controller 322 that can heat the substrate (by means such as a conduction heater or an infrared heater) and/or cool the substrate (by means such as liquid nitrogen or water).

The sputtering operation increases the pressure within the region of the sputtered particles 320. In general, the pressure within the area of the sputtered particles 320 is at least 100 times, and preferably 1000 times, greater than the base pressure.

Referring again to FIG. 6 a cryo pump 324 is preferably used to maintain the base pressure within vacuum chamber 318. In the embodiment depicted, a mechanical pump (dry pump) 326 is operatively connected to the cryo pump 324. Atmosphere from chamber 318 is removed by dry pump 326 at the beginning of the evacuation. At some point, shutter 328 is removed and allows cryo pump 324 to continue the evacuation. A valve 330 controls the flow of atmosphere to dry pump 326 so that it is only open at the beginning of the evacuation.

It is preferred to utilize a substantially constant pumping speed for cryo pump 324, i.e., to maintain a constant outflow of gases through the cryo pump 324. This may be accomplished by sensing the gas outflow via sensor 332 and, as appropriate, varying the extent to which the shutter 328 is open or partially closed.

Without wishing to be bound to any particular theory, applicants believe that the use of a substantially constant gas outflow rate insures a substantially constant deposition of sputtered nitrides.

Referring again to FIG. 6, and in one embodiment thereof, it is preferred to clean the substrate 314 prior to the time it is utilized in the process. Thus, e.g., one may use detergent to clean any grease or oil or fingerprints off the surface of the substrate. Thereafter, one may use an organic solvent such as acetone, isopropryl alcohol, toluene, etc.

In one embodiment, the cleaned substrate 314 is presputtered by suppressing sputtering of the target 308 and sputtering the surface of the substrate 314.

As will be apparent to those skilled in the art, the process depicted in FIG. 6 may be used to prepare coated substrates 314 comprised of moieties other than doped aluminum nitride.

A Preferred Process for Preparing Nanomagnetic Particles

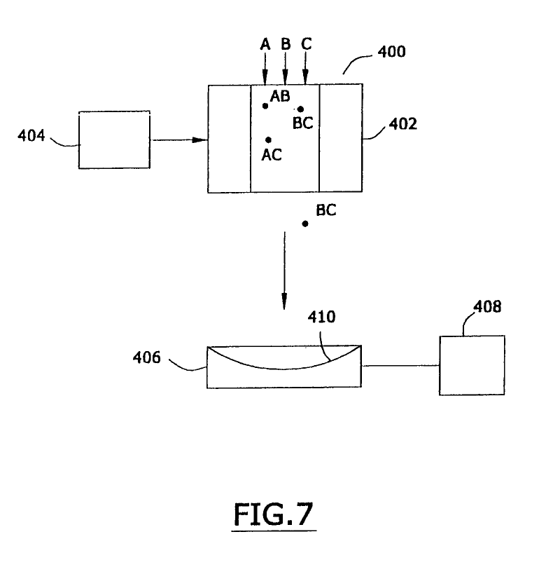

In one embodiment, illustrated in FIG. 7, a substrate is cooled so that nanomagnetic particles are collected on such substrate. Referring to FIG. 7, and in the preferred embodiment depicted therein, a precursor 400 that preferably contains moieties A, B, and C (which are described elsewhere in this specification) are charged to reactor 402.

The reactor 402 may be a plasma reactor. Plasma reactors are described in applicants' U.S. Pat. No. 5,100,868 (process for preparing superconducting films), U.S. Pat. No. 5,120,703 (process for preparing oxide superconducting films by radio-frequency generated aerosol-plasma deposition in atmosphere), U.S. Pat. No. 5,157,015 (process for preparing superconducting films by radio-frequency generated aerosol-plasma deposition in atmosphere), U.S. Pat. No. 5,213,851 (process for preparing ferrite films by radio-frequency generated aerosol plasma deposition in atmosphere), U.S. Pat. No. 5,260,105 (aerosol plasma deposition of films for electrochemical cells), U.S. Pat. No. 5,364,562 (aerosol plasma deposition of insulating oxide powder), U.S. Pat. No. 5,366,770 (aerosol plasma deposition of films for electronic cells), and the like. The entire disclosure of each of these United States patents is hereby incorporated by reference into this specification.

The reactor 402 may be sputtering reactor 300 depicted in FIG. 6.

Referring again to FIG. 7, it will be seen that an energy source 4045 is preferably used in order to cause reaction between moieties A, B, and C. The energy source 404 may be an electromagnetic energy source that supplies energy to the reactor 400. In one embodiment, there are at least two species of moiety A present, and at least two species of moiety C present. The two preferred moiety C species are oxygen and nitrogen.

Within reactor 402 moieties A, B, and C are preferably combined into a metastable state. This metastable state is then caused to travel towards collector 406. Prior to the time it reaches the collector 406, the ABC moiety is formed, either in the reactor 3 and/or between the reactor 402 and the collector 406.

In one embodiment, collector 406 is preferably cooled with a chiller 408 so that its surface 410 is at a temperature below the temperature at which the ABC moiety interacts with surface 410; the goal is to prevent bonding between the ABC moiety and the surface 410. In one embodiment, the surface 410 is at a temperature of less than about 30 degrees Celsius. In another embodiment, the temperature of surface 410 is at the liquid nitrogen temperature, i.e., about 77 degrees Kelvin.

After the ABC moieties have been collected by collector 406, they are removed from surface 410.

FIG. 8 is a schematic illustration of one process of the invention that may be used to make nanomagnetic material. This FIG. 8 is similar in many respects to the FIG. 1 of U.S. Pat. No. 5,213,851, the entire disclosure of which is hereby incorporated by reference into this specification.

Referring to FIG. 8, and in the preferred embodiment depicted therein, it is preferred that the reagents charged into misting chamber 511 will be sufficient, in one embodiment, to form a nano-sized ferrite in the process. The term ferrite, as used in this specification, refers to a material that exhibits ferromagnetism. Ferromagnetism is a property, exhibited by certain metals, alloys, and compounds of the transition (iron group) rare earth and actinide elements, in which the internal magnetic moments spontaneously organize in a common direction; ferromagnetism gives rise to a permeability considerably greater than that of vacuum and to magnetic hysteresis. See, e.g, page 706 of Sybil B. Parker's “McGraw-Hill Dictionary of Scientific and Technical Terms,” Fourth Edition (McGraw-Hill Book Company, New York, N.Y., 1989).

As will be apparent to those skilled in the art, in addition to making nano-sized ferrites by the process depicted in FIG. 8, one may also make other nano-sized materials such as, e.g., nano-sized nitrides and/or nano-sized oxides containing moieties A, B, and C, as is described elsewhere in this specification. For the sake of simplicity of description, and with regard to FIG. 8, a discussion will be had regarding the preparation of ferrites, it being understood that, e.g., and other materials may also be made by such process.

Referring again to FIG. 8, and to the production of ferrites by such process, in one embodiment, the ferromagnetic material contains Fe₂O₃. See, for example, United U.S. Pat. No. 3,576,672 of Harris et al., the entire disclosure of which is hereby incorporated by reference into this specification. As will be apparent, the corresponding nitrides also may be made.

In one embodiment, the ferromagnetic material contains garnet. Pure iron garnet has the formula M₃Fe₅O₁₂; see, e.g., pages 65-256 of Wilhelm H. Von Aulock's “Handbook of Microwave Ferrite Materials” (Academic Press, New York, 1965). Garnet ferrites are also described, e.g., in U.S. Pat. No. 4,721,547, the disclosure of which is hereby incorporated by reference into this specification. As will be apparent, the corresponding nitrides also may be made.

In another embodiment, the ferromagnetic material contains a spinel ferrite. Spinel ferrites usually have the formula MFe₂O₄, wherein M is a divalent metal ion and Fe is a trivalent iron ion. M is typically selected from the group consisting of nickel, zinc, magnesium, manganese, and like. These spinel ferrites are well known and are described, for example, in U.S. Pat. Nos. 5,001,014, 5,000,909, 4,966,625, 4,960,582, 4,957,812, 4,880,599, 4,862,117, 4,855,205, 4,680,130, 4,490,268, 3,822,210, 3,635,898, 3,542,685, 3,421,933, and the like. The disclosure of each of these patents is hereby incorporated by reference into this specification. Reference may also be had to pages 269-406 of the Von Aulock book for a discussion of spinel ferrites. As will be apparent, the corresponding nitrides also may be made.

In yet another embodiment, the ferromagnetic material contains a lithium ferrite. Lithium ferrites are often described by the formula (Li_{0.5}Fe_{0.5})2+(Fe₂)3+O₄. Some illustrative lithium ferrites are described on pages 407-434 of the aforementioned Von Aulock book and in U.S. Pat. Nos. 4,277,356, 4,238,342, 4,177,438, 4,155,963, 4,093,781, 4,067,922, 3,998,757, 3,767,581, 3,640,867, and the like. The disclosure of each of these patents is hereby incorporated by reference into this specification. As will be apparent, the corresponding nitrides also may be made.

In yet another embodiment, the ferromagnetic material contains a hexagonal ferrite. These ferrites are well known and are disclosed on pages 451-518 of the Von Aulock book and also in U.S. Pat. Nos. 4,816,292, 4,189,521, 5,061,586, 5,055,322, 5,051,201, 5,047,290, 5,036,629, 5,034,243, 5,032,931, and the like. The disclosure of each of these patents is hereby incorporated by reference into this specification. As will be apparent, the corresponding nitrides also may be made.

In yet another embodiment, the ferromagnetic material contains one or more of the moieties A, B, and C disclosed in the phase diagram disclosed elsewhere in this specification and discussed elsewhere in this specification.

Referring again to FIG. 8, and in the preferred embodiment depicted therein, it will be appreciated that the solution 509 will preferably comprise reagents necessary to form the required magnetic material. For example, in one embodiment, in order to form the spinel nickel ferrite of the formula NiFe₂O₄, the solution should contain nickel and iron, which may be present in the form of nickel nitrate and iron nitrate. By way of further example, one may use nickel chloride and iron chloride to form the same spinel. By way of further example, one may use nickel sulfate and iron sulfate.

It will be apparent to skilled chemists that many other combinations of reagents, both stoichiometric and nonstoichiometric, may be used in applicants' process to make many different magnetic materials.

In one preferred embodiment, the solution 509 contains the reagent needed to produce a desired ferrite in stoichiometric ratio. Thus, to make the NiFe₂O₄ ferrite in this embodiment, one mole of nickel nitrate may be charged with every two moles of iron nitrate.

In one embodiment, the starting materials are powders with purities exceeding 99 percent.

In one embodiment, compounds of iron and the other desired ions are present in the solution in the stoichiometric ratio.

In one preferred embodiment, ions of nickel, zinc, and iron are present in a stoichiometric ratio of 0.5/0.5/2.0, respectively. In another preferred embodiment, ions of lithium and iron are present in the ratio of 0.5/2.5. In yet another preferred embodiment, ions of magnesium and iron are present in the ratio of 1.0/2.0. In another embodiment, ions of manganese and iron are present in the ratio 1.0/2.0. In yet another embodiment, ions of yttrium and iron are present in the ratio of 3.0/5.0. In yet another embodiment, ions of lanthanum, yttrium, and iron are present in the ratio of 0.5/2.5/5.0. In yet another embodiment, ions of neodymium, yttrium, gadolinium, and iron are present in the ratio of 1.0/1.07/0.93/5.0, or 1.0/1.1/0.9/5.0, or 1/1.12/0.88/5.0. In yet another embodiment, ions of samarium and iron are present in the ratio of 3.0/5.0. In yet another embodiment, ions of neodymium, samarium, and iron are present in the ratio of 0.1/2.9/5.0, or 0.25/2.75/5.0, or 0.375/2.625/5.0. In yet another embodiment, ions of neodymium, erbium, and iron are present in the ratio of 1.5/1.5/5.0. In yet another embodiment, samarium, yttrium, and iron ions are present in the ratio of 0.51/2.49/5.0, or 0.84/2.16/5.0, or 1.5/1.5/5.0. In yet another embodiment, ions of yttrium, gadolinium, and iron are present in the ratio of 2.25/0.75/5.0, or 1.5/1.5/5.0, or 0.75/2.25/5.0. In yet another embodiment, ions of terbium, yttrium, and iron are present in the ratio of 0.8/2.2/5.0, or 1.0/2.0/5.0. In yet another embodiment, ions of dysprosium, aluminum, and iron are present in the ratio of 3/x/5-x, when x is from 0 to 1.0. In yet another embodiment, ions of dysprosium, gallium, and iron are also present in the ratio of 3/x/5-x. In yet another embodiment, ions of dysprosium, chromium, and iron are also present in the ratio of 3/x/5-x.

The ions present in the solution, in one embodiment, may be holmium, yttrium, and iron, present in the ratio of z/3-z/5.0, where z is from about 0 to 1.5.

The ions present in the solution may be erbium, gadolinium, and iron in the ratio of 1.5/1.5/5.0. The ions may be erbium, yttrium, and iron in the ratio of 1.5/1.5/1.5, or 0.5/2.5/5.0.

The ions present in the solution may be thulium, yttrium, and iron, in the ratio of 0.06/2.94/5.0.

The ions present in the solution may be ytterbium, yttrium, and iron, in the ratio of 0.06/2.94/5.0.

The ions present in the solution may be lutetium, yttrium, and iron in the ratio of y/3-y/5.0, wherein y is from 0 to 3.0.

The ions present in the solution may be iron, which can be used to form Fe₆O₈ (two formula units of Fe₃O₄). The ions present may be barium and iron in the ratio of 1.0/6.0, or 2.0/8.0. The ions present may be strontium and iron, in the ratio of 1.0/12.0. The ions present may be strontium, chromium, and iron in the ratio of 1.0/1.0/10.0, or 1.0/6.0/6.0. The ions present may be suitable for producing a ferrite of the formula (Meₓ)₃+Ba₁₋ₓFe₁₂O₁₉, wherein Me is a rare earth selected from the group consisting of lanthanum, promethium, neodymium, samarium, europium, and mixtures thereof.

The ions present in the solution may contain barium, either lanthanum or promethium, iron, and cobalt in the ratio of 1-a/a/12-a/a, wherein a is from 0.0 to 0.8.

The ions present in the solution may contain barium, cobalt, titanium, and iron in the ratio of 1.0/b/b/12-2b, wherein b is from 0.0 to 1.6.

The ions present in the solution may contain barium, nickel or cobalt or zinc, titanium, and iron in the ratio of 1.0/c/c/12-2c, wherein c is from 0.0 to 1.5.

The ions present in the solution may contain barium, iron, iridium, and zinc in the ratio of 1.0/12-2d/d/d, wherein d is from 0.0 to 0.6.

The ions present in the solution may contain barium, nickel, gallium, and iron in the ratio of 1.0/2.0/7.0/9.0, or 1.0/2.0/5.0/11.0. Alternatively, the ions may contain barium, zinc, gallium or aluminum, and iron in the ratio of 1.0/2.0/3.0/13.0.

Each of these ferrites is well known to those in the ferrite art and is described, e.g., in the aforementioned Von Aulock book.

The ions described above are preferably available in solution 509 in water-soluble form, such as, e.g., in the form of water-soluble salts. Thus, e.g., one may use the nitrates or the chlorides or the sulfates or the phosphates of the cations. Other anions which form soluble salts with the cation(s) also may be used.

Alternatively, one may use salts soluble in solvents other than water. Some of these other solvents which may be used to prepare the material include nitric acid, hydrochloric acid, phosphoric acid, sulfuric acid, and the like. As is well known to those skilled in the art, many other suitable solvents may be used; see, e.g., J. A. Riddick et al., “Organic Solvents, Techniques of Chemistry,” Volume II, 3rd edition (Wiley-Interscience, New York, N.Y., 1970).

In one preferred embodiment, where a solvent other than water is used, each of the cations is present in the form of one or more of its oxides. For example, one may dissolve iron oxide in nitric acid, thereby forming a nitrate. For example, one may dissolve zinc oxide in sulfuric acid, thereby forming a sulfate. One may dissolve nickel oxide in hydrochloric acid, thereby forming a chloride. Other means of providing the desired cation(s) will be readily apparent to those skilled in the art.

In general, as long as the desired cation(s) are present in the solution, it is not significant how the solution was prepared.

In general, one may use commercially available reagent grade materials. Thus, by way of illustration and not limitation, one may use the following reagents available in the 1988-1989 Aldrich catalog (Aldrich Chemical Company, Inc., Milwaukee, Wis.): barium chloride, catalog number 31,866-3; barium nitrate, catalog number 32,806-5; barium sulfate, catalog number 20,276-2; strontium chloride hexhydrate, catalog number 20,466-3; strontium nitrate, catalog number 20,449-8; yttrium chloride, catalog number 29,826-3; yttrium nitrate tetrahydrate, catalog number 21,723-9; yttrium sulfate octahydrate, catalog number 20,493-5. This list is merely illustrative, and other compounds that can be used will be readily apparent to those skilled in the art. Thus, any of the desired reagents also may be obtained from the 1989-1990 AESAR catalog (Johnson Matthey/AESAR Group, Seabrook, N. H.), the 1990/1991 Alfa catalog (Johnson Matthey/Alfa Products, Ward Hill, Ma.), the Fisher 88 catalog (Fisher Scientific, Pittsburgh, Pa.), and the like.

As long as the metals present in the desired ferrite material are present in solution 509 in the desired stoichiometry, it does not matter whether they are present in the form of a salt, an oxide, or in another form. In one embodiment, however, it is preferred to have the solution contain either the salts of such metals, or their oxides.

The solution 509 of the compounds of such metals preferably will be at a concentration of from about 0.01 to about 1,000 grams of said reagent compounds per liter of the resultant solution. As used in this specification, the term liter refers to 1,000 cubic centimeters.

In one embodiment, it is preferred that solution 509 have a concentration of from about 1 to about 300 grams per liter and, preferably, from about 25 to about 170 grams per liter. It is even more preferred that the concentration of said solution 9 be from about 100 to about 160 grams per liter. In an even more preferred embodiment, the concentration of said solution 509 is from about 140 to about 160 grams per liter.

In one preferred embodiment, aqueous solutions of nickel nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel nitrate, zinc nitrate, and iron nitrate with purities of at least 99.9 percent are mixed in the molar ratio of 0.5:0.5:2 and then dissolved in distilled water to form a solution with a concentration of 150 grams per liter.

In one preferred embodiment, aqueous solutions of zinc nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel chloride, and iron chloride with purities of at least 99.9 percent are mixed in the molar ratio of 1:2 and then dissolved in distilled water to form a solution with a concentration of 150 grams per liter.

In one preferred embodiment, aqueous solutions of nickel chloride, zinc chloride, and iron chloride with purities of at least 99.9 percent are mixed in the molar ratio of 0.5:0.5:2 and then dissolved in distilled water to form a solution with a concentration of 150 grams per liter.

In one preferred embodiment, aqueous solutions of zinc chloride, and iron chloride with purities of at least 99.9 percent are mixed in the molar ratio of 1:2 and then dissolved in distilled water to form a solution with a concentration of 150 grams per liter.

In one embodiment, mixtures of chlorides and nitrides may be used. Thus, for example, in one preferred embodiment, the solution is comprised of both iron chloride and nickel nitrate in the molar ratio of 2.0/1.0.

Referring again to FIG. 8, and to the preferred embodiment depicted therein, the solution 509 in misting chamber 511 is preferably caused to form into an aerosol, such as a mist.

The term aerosol, as used in this specification, refers to a suspension of ultramicroscopic solid or liquid particles in air or gas, such as smoke, fog, or mist. See, e.g., page 15 of “A dictionary of mining, mineral, and related terms,” edited by Paul W. Thrush (U.S. Department of the Interior, Bureau of Mines, 1968), the disclosure of which is hereby incorporated by reference into this specification.

As used in this specification, the term mist refers to gas-suspended liquid particles which have diameters less than 10 microns.

The aerosol/mist consisting of gas-suspended liquid particles with diameters less than 10 microns may be produced from solution 509 by any conventional means that causes sufficient mechanical disturbance of said solution. Thus, one may use mechanical vibration. In one preferred embodiment, ultrasonic means are used to mist solution 9. As is known to those skilled in the art, by varying the means used to cause such mechanical disturbance, one can also vary the size of the mist particles produced.

As is known to those skilled in the art, ultrasonic sound waves (those having frequencies above 20,000 hertz) may be used to mechanically disturb solutions and cause them to mist. Thus, by way of illustration, one may use the ultrasonic nebulizer sold by the DeVilbiss Health Care, Inc. of Somerset, Pennsylvania; see, e.g., the “Instruction Manual” for the “Ultra-Neb 99 Ultrasonic Nebulizer, publication A-850-C (published by DeVilbiss, Somerset, Pa., 1989).

In the embodiment shown in FIG. 8, the oscillators of ultrasonic nebulizer 513 are shown contacting an exterior surface of misting chamber 511. In this embodiment, the ultrasonic waves produced by the oscillators are transmitted via the walls of the misting chamber 511 and effect the misting of solution 509.

In another embodiment, not shown, the oscillators of ultrasonic nebulizer 513 are in direct contact with solution 509.

In one embodiment, it is preferred that the ultrasonic power used with such machine is in excess of one watt and, more preferably, in excess of 10 watts. In one embodiment, the power used with such machine exceeds about 50 watts.

During the time solution 509 is being caused to mist, it is preferably contacted with carrier gas to apply pressure to the solution and mist. It is preferred that a sufficient amount of carrier gas be introduced into the system at a sufficiently high flow rate so that pressure on the system is in excess of atmospheric pressure. Thus, for example, in one embodiment wherein chamber 511 has a volume of about 200 cubic centimeters, the flow rate of the carrier gas was from about 100 to about 150 milliliters per minute.

In one embodiment, the carrier gas 515 is introduced via feeding line 517 at a rate sufficient to cause solution 509 to mist at a rate of from about 0.5 to about 20 milliliters per minute. In one embodiment, the misting rate of solution 9 is from about 1.0 to about 3.0 milliliters per minute.

Substantially any gas that facilitates the formation of plasma may be used as carrier gas 515. Thus, by way of illustration, one may use oxygen, air, argon, nitrogen, mixtures thereof and the like; in one embodiment, a mixture of oxygen and nitrogen is used. It is preferred that the carrier gas used be a compressed gas under a pressure in excess 760 millimeters of mercury. In this embodiment, the use of the compressed gas facilitates the movement of the mist from the misting chamber 511 to the plasma region 521.

The misting container 511 may be any reaction chamber conventionally used by those skilled in the art and preferably is constructed out of such acid-resistant materials such as glass, plastic, and the like.

The mist from misting chamber 511 is fed via misting outlet line 519 into the plasma region 521 of plasma reactor 525. In plasma reactor 525, the mist is mixed with plasma generated by plasma gas 527 and subjected to radio frequency radiation provided by a radio-frequency coil 529.

The plasma reactor 525 provides energy to form plasma and to cause the plasma to react with the mist. Any of the plasma reactors well known to those skilled in the art may be used as plasma reactor 525. Some of these plasma reactors are described in J. Mort et al.'s “Plasma Deposited Thin Films” (CRC Press Inc., Boca Raton, Fla., 1986); in “Methods of Experimental Physics,” Volume 9—Parts A and B, Plasma Physics (Academic Press, New York, 1970/1971); and in N. H. Burlingame's “Glow Discharge Nitriding of Oxides,” Ph.D. thesis (Alfred University, Alfred, N.Y., 1985), available from University Microfilm International, Ann Arbor, Mich.

In one preferred embodiment, the plasma reactor 525 is a “model 56 torch” available from the TAFA Inc. of Concord, N.H. It is preferably operated at a frequency of about 4 megahertz and an input power of 30 kilowatts.

Referring again to FIG. 8, and to the preferred embodiment depicted therein, it will be seen that into feeding lines 529 and 531 is fed plasma gas 527. As is known to those skilled in the art, a plasma can be produced by passing gas into a plasma reactor. A discussion of the formation of plasma is contained in B. Chapman's “Glow Discharge Processes” (John Wiley & Sons, New York, 1980).

In one preferred embodiment, the plasma gas used is a mixture of argon and oxygen. In another embodiment, the plasma gas is a mixture of nitrogen and oxygen. In yet another embodiment, the plasma gas is pure argon or pure nitrogen.

When the plasma gas is pure argon or pure nitrogen, it is preferred to introduce into the plasma reactor at a flow rate of from about 5 to about 30 liters per minute.

When a mixture of oxygen and either argon or nitrogen is used, the concentration of oxygen in the mixture preferably is from about 1 to about 40 volume percent and, more preferably, from about 15 to about 25 volume percent. When such a mixture is used, the flow rates of each gas in the mixture should be adjusted to obtain the desired gas concentrations. Thus, by way of illustration, in one embodiment that uses a mixture of argon and oxygen, the argon flow rate is 15 liters per minute, and the oxygen flow rate is 40 liters per minute.

In one embodiment, auxiliary oxygen 533 is fed into the top of reactor 25, between the plasma region 521 and the flame region 540, via lines 536 and 538. In this embodiment, the auxiliary oxygen is not involved in the formation of plasma but is involved in the enhancement of the oxidation of the ferrite material.

Radio frequency energy is applied to the reagents in the plasma reactor 525, and it causes vaporization of the mist.

In general, the energy is applied at a frequency of from about 100 to about 30,000 kilohertz. In one embodiment, the radio frequency used is from about 1 to 20 megahertz. In another embodiment, the radio frequency used is from about 3 to about 5 megahertz.

As is known to those skilled in the art, such radio frequency alternating currents may be produced by conventional radio frequency generators. Thus, by way of illustration, said TAPA Inc. “model 56 torch” may be attached to a radio frequency generator rated for operation at 35 kilowatts which manufactured by Lepel Company (a division of TAFA Inc.) and which generates an alternating current with a frequency of 4 megahertz at a power input of 30 kilowatts. Thus, e.g., one may use an induction coil driven at 2.5-5.0 megahertz that is sold as the “PLASMOC 2” by ENI Power Systems, Inc. of Rochester, N.Y.

The use of these type of radio-frequency generators is described in the Ph.D. theses entitled (1) “Heat Transfer Mechanisms in High-Temperature Plasma Processing of Glasses,” Donald M. McPherson (Alfred University, Alfred, N.Y., January, 1988) and (2) the aforementioned Nicholas H. Burlingame's “Glow Discharge Nitriding of Oxides.”

The plasma vapor 523 formed in plasma reactor 525 is allowed to exit via the aperture 542 and can be visualized in the flame region 540. In this region, the plasma contacts air that is at a lower temperature than the plasma region 521, and a flame is visible. A theoretical model of the plasma/flame is presented on pages 88 et seq. of said McPherson thesis.

The vapor 544 present in flame region 540 is propelled upward towards substrate 546. Any material onto which vapor 544 will condense may be used as a substrate. Thus, by way of illustration, one may use nonmagnetic materials such alumina, glass, gold-plated ceramic materials, and the like. In one embodiment, substrate 46 consists essentially of a magnesium oxide material such as single crystal magnesium oxide, polycrystalline magnesium oxide, and the like.

In another embodiment, the substrate 546 consists essentially of zirconia such as, e.g., yttrium stabilized cubic zirconia.

In another embodiment, the substrate 546 consists essentially of a material selected from the group consisting of strontium titanate, stainless steel, alumina, sapphire, and the like.

The aforementioned listing of substrates is merely meant to be illustrative, and it will be apparent that many other substrates may be used. Thus, by way of illustration, one may use any of the substrates mentioned in M. Sayer's “Ceramic Thin Films . . . ” article, supra. Thus, for example, in one embodiment it is preferred to use one or more of the substrates described on page 286 of “Superconducting Devices,” edited by S. T. Ruggiero et al. (Academic Press, Inc., Boston, 1990).

One advantage of this embodiment of applicants' process is that the substrate may be of substantially any size or shape, and it may be stationary or movable. Because of the speed of the coating process, the substrate 546 may be moved across the aperture 542 and have any or all of its surface coated.

As will be apparent to those skilled in the art, in the embodiment depicted in FIG. 8, the substrate 546 and the coating 548 are not drawn to scale but have been enlarged to the sake of ease of representation.

Referring again to FIG. 8, the substrate 546 may be at ambient temperature. Alternatively, one may use additional heating means to heat the substrate prior to, during, or after deposition of the coating.

Referring again to FIG. 8, and in one preferred embodiment, a heater (not shown) is used to heat the substrate to a temperature of from about 100 to about 800 degrees centigrade.

In one aspect of this embodiment, temperature sensing means (not shown) may be used to sense the temperature of the substrate and, by feedback means (not shown), adjust the output of the heater (not shown). In one embodiment, not shown, when the substrate 46 is relatively near flame region 40, optical pyrometry measurement means (not shown) may be used to measure the temperature near the substrate.

In one embodiment, a shutter (not shown) is used to selectively interrupt the flow of vapor 544 to substrate 546. This shutter, when used, should be used prior to the time the flame region has become stable; and the vapor should preferably not be allowed to impinge upon the substrate prior to such time.

The substrate 546 may be moved in a plane that is substantially parallel to the top of plasma chamber 525. Alternatively, or additionally, it may be moved in a plane that is substantially perpendicular to the top of plasma chamber 525. In one embodiment, the substrate 46 is moved stepwise along a predetermined path to coat the substrate only at certain predetermined areas.

In one embodiment, rotary substrate motion is utilized to expose as much of the surface of a complex-shaped article to the coating. This rotary substrate motion may be effectuated by conventional means. See, e.g., “Physical Vapor Deposition,” edited by Russell J. Hill (Temescal Division of The BOC Group, Inc., Berkeley, Calif., 1986).

The process of this embodiment of the invention allows one to coat an article at a deposition rate of from about 0.01 to about 10 microns per minute and, preferably, from about 0.1 to about 1.0 microns per minute, with a substrate with an exposed surface of 35 square centimeters. One may determine the thickness of the film coated upon said reference substrate material (with an exposed surface of 35 square centimeters) by means well known to those skilled in the art.

The film thickness can be monitored in situ, while the vapor is being deposited onto the substrate. Thus, by way of illustration, one may use an IC-6000 thin film thickness monitor (also referred to as “deposition controller”) manufactured by Leybold Inficon Inc. of East Syracuse, N.Y.

The deposit formed on the substrate may be measured after the deposition by standard profilometry techniques. Thus, e.g., one may use a DEKTAK Surface Profiler, model number 900051 (available from Sloan Technology Corporation, Santa Barbara, Calif.).

In general, at least about 80 volume percent of the particles in the as-deposited film are smaller than about 1 micron. It is preferred that at least about 90 percent of such particles are smaller than 1 micron. Because of this fine grain size, the surface of the film is relatively smooth.

In one preferred embodiment, the as-deposited film is post-annealed.

It is preferred that the generation of the vapor in plasma rector 525 be conducted under substantially atmospheric pressure conditions. As used in this specification, the term “substantially atmospheric” refers to a pressure of at least about 600 millimeters of mercury and, preferably, from about 600 to about 1,000 millimeters of mercury. It is preferred that the vapor generation occur at about atmospheric pressure. As is well known to those skilled in the art, atmospheric pressure at sea level is 760 millimeters of mercury.

The process of this invention may be used to produce coatings on a flexible substrate such as, e.g., stainless steel strips, silver strips, gold strips, copper strips, aluminum strips, and the like. One may deposit the coating directly onto such a strip. Alternatively, one may first deposit one or more buffer layers onto the strip(s). In other embodiments, the process of this invention may be used to produce coatings on a rigid or flexible cylindrical substrate, such as a tube, a rod, or a sleeve.

Referring again to FIG. 8, and in the embodiment depicted therein, as the coating 548 is being deposited onto the substrate 546, and as it is undergoing solidification thereon, it is preferably subjected to a magnetic field produced by magnetic field generator 550.

In this embodiment, it is preferred that the magnetic field produced by the magnetic field generator 550 have a field strength of from about 2 Gauss to about 40 Tesla.

It is preferred to expose the deposited material for at least 10 seconds and, more preferably, for at least 30 seconds, to the magnetic field, until the magnetic moments of the nano-sized particles being deposited have been substantially aligned.

As used herein, the term “substantially aligned” means that the inductance of the device being formed by the deposited nano-sized particles is at least 90 percent of its maximum inductance. One may determine when such particles have been aligned by, e.g., measuring the inductance, the permeability, and/or the hysteresis loop of the deposited material.

Thus, e.g., one may measure the degree of alignment of the deposited particles with an impedance meter, an inductance meter, or a SQUID.

In one embodiment, the degree of alignment of the deposited particles is measured with an inductance meter. One may use, e.g., a conventional conductance meter such as, e.g., the conductance meters disclosed in U.S. Pat. Nos. 4,779,462, 4,937,995, 5,728,814 (apparatus for determining and recording injection does in syringes using electrical inductance), U.S. Pat. Nos. 6,318,176, 5,014,012, 4,869,598, 4,258,315 (inductance meter), U.S. Pat. No. 4,045,728 (direct reading inductance meter), U.S. Pat. Nos. 6,252,923, 6,194,898, 6,006,023 (molecular sensing apparatus), U.S. Pat. No. 6,048,692 (sensors for electrically sensing binding events for supported molecular receptors), and the like. The entire disclosure of each of these United States patents is hereby incorporated by reference into this specification.

When measuring the inductance of the coated sample, the inductance is preferably measured using an applied wave with a specified frequency. As the magnetic moments of the coated samples align, the inductance increases until a specified value; and it rises in accordance with a specified time constant in the measurement circuitry.

In one embodiment, the deposited material is contacted with the magnetic field until the inductance of the deposited material is at least about 90 percent of its maximum value under the measurement circuitry. At this time, the magnetic particles in the deposited material have been aligned to at least about 90 percent of the maximum extent possible for maximizing the inductance of the sample.

By way of illustration and not limitation, a metal rod with a diameter of 1 micron and a length of 1 millimeter, when uncoated with magnetic nano-sized particles, might have an inductance of about 1 nanohenry. When this metal rod is coated with, e.g., nano-sized ferrites, then the inductance of the coated rod might be 5 nanohenries or more. When the magnetic moments of the coating are aligned, then the inductance might increase to 50 nanohenries, or more. As will be apparent to those skilled in the art, the inductance of the coated article will vary, e.g., with the shape of the article and also with the frequency of the applied electromagnetic field.

One may use any of the conventional magnetic field generators known to those skilled in the art to produce such as magnetic field. Thus, e.g., one may use one or more of the magnetic field generators disclosed in U.S. Pat. Nos. 6,503,364, 6,377,149 (magnetic field generator for magnetron plasma generation), U.S. Pat. No. 6,353,375 (magnetostatic wave device), U.S. Pat. No. 6,340,888 (magnetic field generator for MRI), U.S. Pat. Nos. 6,336,989, 6,335,617 (device for calibrating a magnetic field generator), U.S. Pat. Nos. 6,313,632, 6,297,634, 6,275,128, 6,246,066 (magnetic field generator and charged particle beam irradiator), U.S. Pat. No. 6,114,929 (magnetostatic wave device), U.S. Pat. No. 6,099,459 (magnetic field generating device and method of generating and applying a magnetic field), U.S. Pat. Nos. 5,795,212, 6,106,380 (deterministic magnetorheological finishing), U.S. Pat. No. 5,839,944 (apparatus for deterministic magnetorheological finishing), U.S. Pat. No. 5,971,835 (system for abrasive jet shaping and polishing of a surface using a magnetorheological fluid), U.S. Pat. Nos. 5,951,369, 6,506,102 (system for magnetorheological finishing of substrates), U.S. Pat. No. 6,267,651, 6,309,285 (magnetic wiper), and the like. The entire disclosure of each of these United States patents is hereby incorporated by reference into this specification.

In one embodiment, the magnetic field is 1.8 Tesla or less. In this embodiment, the magnetic field can be applied with, e.g., electromagnets disposed around a coated substrate.

For fields greater than about 2 Tesla, one may use superconducting magnets that produce fields as high as 40 Tesla. Reference may be had, e.g., to U.S. Pat. No. 5,319,333 (superconducting homogeneous high field magnetic coil), U.S. Pat. Nos. 4,689,563, 6,496,091 (superconducting magnet arrangement), U.S. Pat. No. 6,140,900 (asymmetric superconducting magnets for magnetic resonance imaging), U.S. Pat. No. 6,476,700 (superconducting magnet system), U.S. Pat. No. 4,763,404 (low current superconducting magnet), U.S. Pat. No. 6,172,587 (superconducting high field magnet), U.S. Pat. No. 5,406,204, and the like. The entire disclosure of each of these United States patents is hereby incorporated by reference into this specification.

In one embodiment, no magnetic field is applied to the deposited coating while it is being solidified. In this embodiment, as will be apparent to those skilled in the art, there still may be some alignment of the magnetic domains in a plane parallel to the surface of substrate as the deposited particles are locked into place in a matrix (binder) deposited onto the surface.

In one embodiment, depicted in FIG. 8, the magnetic field 552 is preferably delivered to the coating 548 in a direction that is substantially parallel to the surface 556 of the substrate 546. In another embodiment, not shown, the magnetic field 558 is delivered in a direction that is substantially perpendicular to the surface 556. In yet another embodiment, the magnetic field 560 is delivered in a direction that is angularly disposed vis-a-vis surface 556 and may form, e.g., an obtuse angle (as in the case of field 62). As will be apparent, combinations of these magnetic fields may be used.

FIG. 9 is a flow diagram of another process that may be used to make the nanomagnetic compositions of this invention. Referring to FIG. 9, and to the preferred process depicted therein, it will be seen that nano-sized ferromagnetic material(s), with a particle size less than about 100 nanometers, is preferably charged via line 660 to mixer 663. It is preferred to charge a sufficient amount of such nano-sized material(s) so that at least about 10 weight percent of the mixture formed in mixer 663 is comprised of such nano-sized material. In one embodiment, at least about 40 weight percent of such mixture in mixer 663 is comprised of such nano-sized material. In another embodiment, at least about 50 weight percent of such mixture in mixer 663 is comprised of such nano-sized material.

In one embodiment, one or more binder materials are charged via line 664 to mixer 662. In one embodiment, the binder used is a ceramic binder. These ceramic binders are well known. Reference may be had, e.g., to pages 172-197 of James S. Reed's “Principles of Ceramic Processing,” Second Edition (John Wiley & Sons, Inc., New York, N.Y., 1995). As is disclosed in the Reed book, the binder may be a clay binder (such as fine kaolin, ball clay, and bentonite), an organic colloidal particle binder (such as microcrystalline cellulose), a molecular organic binder (such as natural gums, polyscaccharides, lignin extracts, refined alginate, cellulose ethers, polyvinyl alcohol, polyvinylbutyral, polymethyl methacrylate, polyethylene glycol, paraffin, and the like.). etc.

In one embodiment, the binder is a synthetic polymeric or inorganic composition. Thus, and referring to George S. Brady et al.'s “Materials Handbook,” (McGraw-Hill, Inc., New York, N.Y. 1991), the binder may be acrylonitrile-butadiene-styrene (see pages 5-6), an acetal resin (see pages 6-7), an acrylic resin (see pages 10-12), an adhesive composition (see pages 14-18), an alkyd resin (see page 27-28), an allyl plastic (see pages 31-32), an amorphous metal (see pages 53-54), a biocompatible material (see pages 95-98), boron carbide (see page 106), boron nitride (see page 107), camphor (see page 135), one or more carbohydrates (see pages 138-140), carbon steel (see pages 146-151), casein plastic (see page 157), cast iron (see pages 159-164), cast steel (see pages 166-168), cellulose (see pages 172-175), cellulose acetate (see pages 175-177), cellulose nitrate (see pages 177), cement (see page 178-180), ceramics (see pages 180-182), cermets (see pages 182-184), chlorinated polyethers (see pages 191-191), chlorinated rubber (see pages 191-193), cold-molded plastics (see pages 220-221), concrete (see pages 225-227), conductive polymers and elastomers (see pages 227-228), degradable plastics (see pages 261-262), dispersion-strengthened metals (see pages 273-274), elastomers (see pages 284-290), enamel (see pages 299-301), epoxy resins (see pages 301-302), expansive metal (see page 313), ferrosilicon (see page 327), fiber-reinforced plastics (see pages 334-335), fluoroplastics (see pages 345-347), foam materials (see pages 349-351), fusible alloys (see pages 362-364), glass (see pages 376-383), glass-ceramic materials (see pages 383-384), gypsum (see pages 406-407), impregnated wood (see pages 422-423), latex (see pages 456-457), liquid crystals (see page 479). lubricating grease (see pages 488-492), magnetic materials (see pages 505-509), melamine resin (see pages 5210-521), metallic materials (see pages 522-524), nylon (see pages 567-569), olefin copolymers (see pages 574-576), phenol-formaldehyde resin (see pages 615-617), plastics (see pages 637-639), polyarylates (see pages 647-648), polycarbonate resins (see pages 648), polyester thermoplastic resins (see pages 648-650), polyester thermosetting resins (see pages 650-651), polyethylenes (see pages 651-654), polyphenylene oxide (see pages 644-655), polypropylene plastics (see pages 655-656), polystyrenes (see pages 656-658), proteins (see pages 666-670), refractories (see pages 691-697), resins (see pages 697-698), rubber (see pages 706-708), silicones (see pages 747-749), starch (see pages 797-802), superalloys (see pages 819-822), superpolymers (see pages 823-825), thermoplastic elastomers (see pages 837-839), urethanes (see pages 874-875), vinyl resins (see pages 885-888), wood (see pages 912-916), mixtures thereof, and the like.

Referring again to FIG. 9, one may charge to line 664 either one or more of these “binder material(s)” and/or the precursor(s) of these materials that, when subjected to the appropriate conditions in former 666, will form the desired mixture of nanomagnetic material and binder.

Referring again to FIG. 9, and in the preferred process depicted therein, the mixture within mixer 63 is preferably stirred until a substantially homogeneous mixture is formed. Thereafter, it may be discharged via line 665 to former 66.

One process for making a fluid composition comprising nanomagnetic particles is disclosed in U.S. Pat. No. 5,804,095, “Magnetorheological Fluid Composition,” of Jacobs et al; the disclosure of this patent is incorporated herein by reference. In this patent, there is disclosed a process comprising numerous material handling steps used to prepare a nanomagnetic fluid comprising iron carbonyl particles. One suitable source of iron carbonyl particles having a median particle size of 3.1 microns is the GAF Corporation.

The process of Jacobs et al, is applicable to the present invention, wherein such nanomagnetic fluid further comprises a polymer binder, thereby forming a nanomagnetic paint. In one embodiment, the nanomagnetic paint is formulated without abrasive particles of cerium dioxide. In another embodiment, the nanomagnetic fluid further comprises a polymer binder, and aluminum nitride is substituted for cerium dioxide.

There are many suitable mixing processes and apparatus for the milling, particle size reduction, and mixing of fluids comprising solid particles. For example, e.g., iron carbonyl particles or other ferromagnetic particles of the paint may be further reduced to a size on the order of 100 nanometers or less, and/or thoroughly mixed with a binder polymer and/or a liquid solvent by the use of a ball mill, a sand mill, a paint shaker holding a vessel containing the paint components and hard steel or ceramic beads; a homogenizer (such as the Model Ytron Z made by the Ytron Quadro Corporation of Chesham, United Kingdom, or the Microfluidics M700 made by the MFIC Corporation of Newton, Ma.), a powder dispersing mixer (such as the Ytron Zyclon mixer, or the Ytron Xyclon mixer, or the Ytron PID mixer by the Ytron Quadro Corporation); a grinding mill (such as the Model F10 Mill by the Ytron Quadro Corporation); high shear mixers (such as the Ytron Y mixer by the Ytron Quadro Corporation), the Silverson Laboratory Mixer sold by the Silverson Corporation of East Longmeadow, Ma., and the like. The use of one or more of these apparatus in series or in parallel may produce a suitably formulated nanomagnetic paint.

Referring again to FIG. 9, the former 666 is preferably equipped with an input line 68 and an exhaust line 670 so that the atmosphere within the former can be controlled. One may utilize an ambient atmosphere, an inert atmosphere, pure nitrogen, pure oxygen, mixtures of various gases, and the like. Alternatively, or additionally, one may use lines 668 and 670 to afford subatmospheric pressure, atmospheric pressure, or superatomspheric pressure within former 666.

In the embodiment depicted, former 666 is also preferably comprised of an electromagnetic coil 672 that, in response from signals from controller 674, can control the extent to which, if any, a magnetic field is applied to the mixture within the former 666 (and also within the mold 667 and/or the spinnerette 669).

The controller 674 is also adapted to control the temperature within the former 666 by means of heating/cooling assembly.

Referring again to FIG. 8, and in one preferred embodiment, a heater (not shown) is used to heat the substrate 546 to a temperature of from about 100 to about 800 degrees centigrade.

In one aspect of this embodiment, temperature sensing means (not shown) may be used to sense the temperature of the substrate 546 and, by feedback means (not shown), adjust the output of the heater (not shown). In one embodiment, not shown, when the substrate 546 is relatively near flame region 540, optical pyrometry measurement means (not shown) may be used to measure the temperature near the substrate.

In one embodiment, a shutter (not shown) is used to selectively interrupt the flow of vapor 544 to substrate 546. This shutter, when used, should be used prior to the time the flame region has become stable; and the vapor should preferably not be allowed to impinge upon the substrate prior to such time.

The substrate 546 may be moved in a plane that is substantially parallel to the top of plasma chamber 525. Alternatively, or additionally, it may be moved in a plane that is substantially perpendicular to the top of plasma chamber 525. In one embodiment, the substrate 546 is moved stepwise along a predetermined path to coat the substrate only at certain predetermined areas.

In one embodiment, rotary substrate motion is utilized to expose as much of the surface of a complex-shaped article to the coating. This rotary substrate motion may be effectuated by conventional means. See, e.g., “Physical Vapor Deposition,” edited by Russell J. Hill (Temescal Division of The BOC Group, Inc., Berkeley, Calif., 1986).

The process of this embodiment of the invention allows one to coat an article at a deposition rate of from about 0.01 to about 10 microns per minute and, preferably, from about 0.1 to about 1.0 microns per minute, with a substrate with an exposed surface of 35 square centimeters. One may determine the thickness of the film coated upon said reference substrate material (with an exposed surface of 35 square centimeters) by means well known to those skilled in the art.

The film thickness can be monitored in situ, while the vapor is being deposited onto the substrate. Thus, by way of illustration, one may use an IC-6000 thin film thickness monitor (also referred to as “deposition controller”) manufactured by Leybold Inficon Inc. of East Syracuse, N.Y.

The deposit formed on the substrate may be measured after the deposition by standard profilometry techniques. Thus, e.g., one may use a DEKTAK Surface Profiler, model number 900051 (available from Sloan Technology Corporation, Santa Barbara, Calif.).

In general, at least about 80 volume percent of the particles in the as-deposited film are smaller than about 1 micron. It is preferred that at least about 90 percent of such particles are smaller than 1 micron. Because of this fine grain size, the surface of the film is relatively smooth.

In one preferred embodiment, the as-deposited film is post-annealed.

It is preferred that the generation of the vapor in plasma rector 525 be conducted under substantially atmospheric pressure conditions. As used in this specification, the term “substantially atmospheric” refers to a pressure of at least about 600 millimeters of mercury and, preferably, from about 600 to about 1,000 millimeters of mercury. It is preferred that the vapor generation occur at about atmospheric pressure. As is well known to those skilled in the art, atmospheric pressure at sea level is 760 millimeters of mercury.

The process of this invention may be used to produce coatings on a flexible substrate such as, e.g., stainless steel strips, silver strips, gold strips, copper strips, aluminum strips, and the like. One may deposit the coating directly onto such a strip. Alternatively, one may first deposit one or more buffer layers onto the strip(s). In other embodiments, the process of this invention may be used to produce coatings on a rigid or flexible cylindrical substrate, such as a tube, a rod, or a sleeve.

Referring again to FIG. 8, and in the embodiment depicted therein, as the coating 548 is being deposited onto the substrate 546, and as it is undergoing solidification thereon, it is preferably subjected to a magnetic field produced by magnetic field generator 550.

In this embodiment, it is preferred that the magnetic field produced by the magnetic field generator 550 have a field strength of from about 2 Gauss to about 40 Tesla.

Substrates with Composite Coatings Disposed Thereon

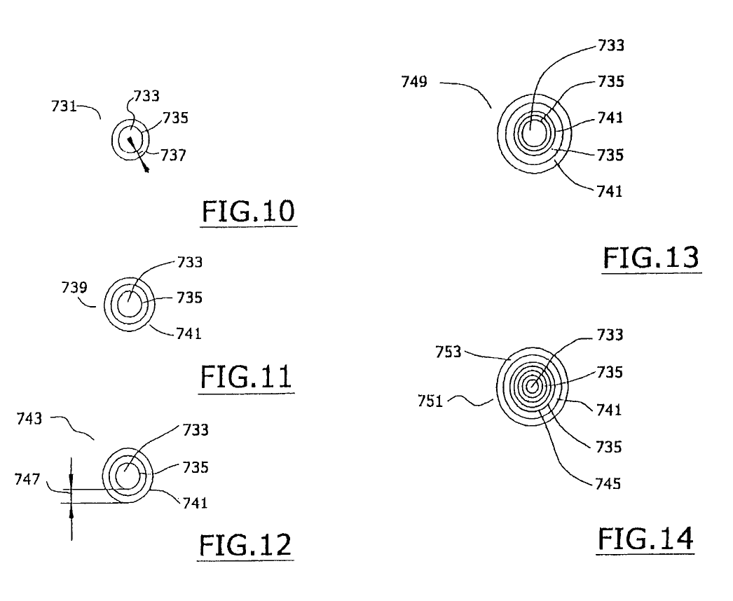

FIGS. 10-14 are sectional views of coated substrates wherein the coatings comprise two more discrete layers of different materials.

FIG. 10 is a sectional view one preferred coated assembly 731 that is comprised of a conductor 733 and, disposed around such conductor 733, a layer of nanomagnetic material 735.

In the embodiment depicted in FIG. 10, the layer 735 of nanomagnetic material preferably has a thickness of at least 150 nanometers and, more preferably, at least about 200 nanometers. In one embodiment, the thickness of layer 735 is from about 500 to about 1,000 nanometers.

FIG. 11 is a schematic sectional view of a magnetically shielded assembly 739 that is similar to assembly 731 but differs therefrom in that a layer 741 of nanoelectrical material is disposed around layer 735.

The layer of nanoelectrical material 741 preferably has a thickness of from about 0.5 to about 2 microns. In this embodiment, the nanoelectrical material comprising layer 741 has a resistivity of from about 1 to about 100 microohm-centimeters. As is known to those skilled in the art, when nanoelectrical material is exposed to electromagnetic radiation, and in particular to an electric field, it will shield the substrate over which it is disposed from such electrical field. Reference may be had, e.g., to International patent publication WO9820719 in which reference is made to U.S. Pat. No. 4,963,291; each of these patents and patent applications is hereby incorporated by reference into this specification.

As is disclosed in U.S. Pat. No. 4,963,291, one may produce electromagnetic shielding resins comprised of electroconductive particles, such as iron, aluminum, copper, silver and steel in sizes ranging from 0.5 to 0.50 microns. The entire disclosure of this United States patent is hereby incorporated by reference into this specification.

The nanoelectrical particles used in this aspect of the invention preferably have a particle size within the range of from about 1 to about 100 microns, and a resistivity of from about 1.6 to about 100 microohm-centimeters. In one embodiment, such nanoelectrical particles comprise a mixture of iron and aluminum. In another embodiment, such nanoelectrical particles consist essentially of a mixture of iron and aluminum.

It is preferred that, in such nanoelectrical particles, and in one embodiment, at least 9 moles of aluminum are present for each mole of iron. In another embodiment, at least about 9.5 moles of aluminum are present for each mole of iron. In yet another embodiment, at least 9.9 moles of aluminum are present for each mole of iron.

In one embodiment, and referring again to FIG. 13, the layer 741 of nanoelectrical material has a thermal conductivity of from about 1 to about 4 watts/centimeter-degree Kelvin.

In one embodiment, not shown, in either or both of layers 735 and 741 there is present both the nanoelectrical material and the nanomagnetic material One may produce such a layer 735 and/or 741 by simultaneously depositing the nanoelectrical particles and the nanomagnetic particles with, e.g., sputtering technology such as, e.g., the sputtering technology described elsewhere in this specification.

FIG. 12 is a sectional schematic view of a magnetically shielded assembly 743 that differs from assembly 731 in that it contains a layer 745 of nanothermal material disposed around the layer 735 of nanomagnetic material. The layer 745 of nanothermal material preferably has a thickness of less than 2 microns and a thermal conductivity of at least about 150 watts/meter-degree Kelvin and, more preferably, at least about 200 watts/meter-degree Kelvin. It is preferred that the resistivity of layer 745 be at least about 10¹⁰ microohm-centimeters and, more preferably, at least about 10¹² microohm-centimeters. In one embodiment, the resistivity of layer 745 is at least about 10¹³ microohm centimeters. In one embodiment, the nanothermal layer is comprised of AlN.

In one embodiment, depicted in FIG. 12, the thickness 747 of all of the layers of material coated onto the conductor 733 is preferably less than about 20 microns.

In FIG. 13, a sectional view of an assembly 749 is depicted that contains, disposed around conductor 733, layers of nanomagnetic material 735, nanoelectrical material 741, nanomagnetic material 735, and nanoelectrical material 741.

In FIG. 14, a sectional view of an assembly 751 is depicted that contains, disposed around conductor 733, a layer 735 of nanomagnetic material, a layer 741 of nanoelectrical material, a layer 735 of nanomagnetic material, a layer 745 of nanothermal material, and a layer 735 of nanomagnetic material. Optionally disposed in layer 753 is antithrombogenic material that is biocompatible with the living organism in which the assembly 751 is preferably disposed.

In the embodiments depicted in FIGS. 10 through 14, the coatings 735, and/or 741, and/or 745, and/or 753, are disposed around a conductor 733. In one embodiment, the conductor so coated is preferably part of medical device, preferably an implanted medical device (such as, e.g., a pacemaker). In another embodiment, in addition to coating the conductor 733, or instead of coating the conductor 733, the actual medical device itself is coated.

Preparation of Coatings Comprised of Nanoelectrical Material

In this portion of the specification, coatings comprised of nanoelectrical material will be described. In accordance with one aspect of this invention, there is provided a nanoelectrical material with an average particle size of less than 100 nanometers, a surface area to volume ratio of from about 0.1 to about 0.05 l/nanometer, and a relative dielectric constant of less than about 1.5.

The nanoelectrical particles of this aspect of the invention have an average particle size of less than about 100 nanometers. In one embodiment, such particles have an average particle size of less than about 50 nanometers. In yet another embodiment, such particles have an average particle size of less than about 10 nanometers.

The nanoelectrical particles of this invention have surface area to volume ratio of from about 0.1 to about 0.05 l/nanometer.

When the nanoelectrical particles of this invention are agglomerated into a cluster, or when they are deposited onto a substrate, the collection of particles preferably has a relative dielectric constant of less than about 1.5. In one embodiment, such relative dielectric constant is less than about 1.2.

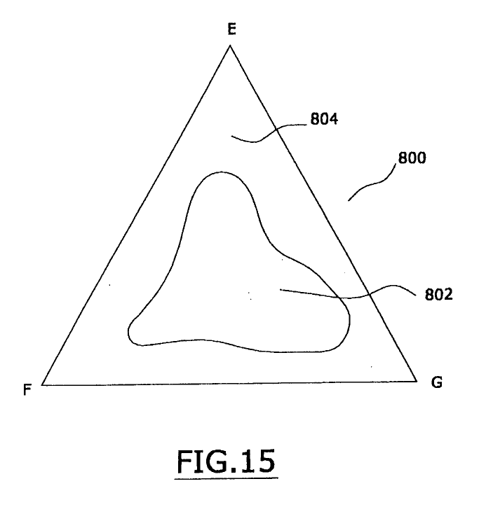

In one embodiment, the nanoelectrical particles of this invention are preferably comprised of aluminum, magnesium, and nitrogen atoms. This embodiment is illustrated in FIG. 15.

FIG. 15 illustrates a phase diagram 800 comprised of moieties E, F, and G. Moiety E is preferably selected from the group consisting of aluminum, copper, gold, silver, and mixtures thereof. It is preferred that the moiety E have a resistivity of from about 2 to about 100 microohm-centimeters. In one preferred embodiment, moiety E is aluminum with a resistivity of about 2.824 microohm-centimeters. As will apparent, other materials with resistivities within the desired range also may be used.

Referring again to FIG. 15, moiety G is selected from the group consisting of nitrogen, oxygen, and mixtures thereof. In one embodiment, C is nitrogen, A is aluminum, and aluminum nitride is present as a phase in the system.

Referring again to FIG. 15, and in one embodiment, moiety F is preferably a dopant that is present in a minor amount in the preferred aluminum nitride. In general, less than about 50 percent (by weight) of the F moiety is present, by total weight of the doped aluminum nitride. In one aspect of this embodiment, less than about 10 weight percent of the F moiety is present, by total weight of the doped aluminum nitride.

The F moiety may be, e.g., magnesium, zinc, tin, indium, gallium, niobium, zirconium, strontium, lanthanum, tungsten, mixtures thereof, and the like. In one embodiment, F is selected from the group consisting of magnesium, zinc, tin, and indium. In another especially preferred embodiment, the F moiety is magnesium.

Referring again to FIG. 15, and when E is aluminum, F is magnesium, and G is nitrogen, it will be seen that regions 802 and 804 correspond to materials which have a low relative dielectric constant (less than about 1.5), and a high relative dielectric constant (greater than about 1.5), respectively.

A Preferred Drug Delivery Assembly

In this section of the specification, applicants will describe a medical device with improved drug delivery capabilities. This medical device is similar to the medical device disclosed in published U.S. patent application 2004/0030379, the entire disclosure of which is hereby incorporated by reference into this specification. However, because applicants use an improved form of magnetic particles in the device, applicants device provides superior magnetic performance and, additionally, superior MRI imageability.

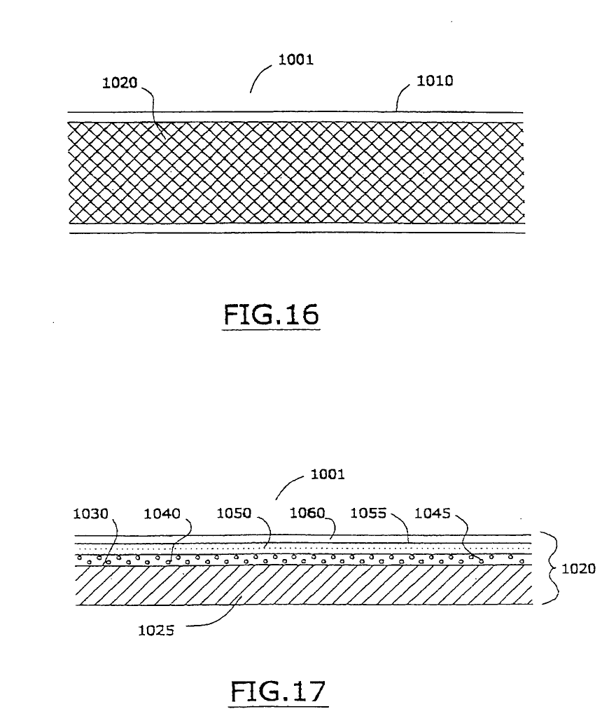

The medical system described in this section of the specification is preferably a stent 1010 (see FIG. 16) comprised of wire like struts 1020 (also see FIG. 16). As is disclosed in paragraph 22 of published U.S. patent application 2004/0030379, “The system of the present invention comprises (1) a medical device having a coating containing a biologically active material, and (2) a source of electromagnetic energy or a source for generating an electromagnetic field. The present invention can facilitate and/or modulate the delivery of the biologically active material from the medical device. The release of the biologically active material from the medical device is facilitated or modulated by the electromagnetic energy source or field. To utilize the system of the present invention, the practitioner may implant the coated medical device using regular procedures. After implantation, the patient is exposed to an extracorporal or external electromagnetic energy source or field to facilitate the release of the biologically active material from the medical device. The delivery of the biologically active material is on-demand, i.e., the material is not delivered or released from the medical device until a practitioner determines that the patient is in need of the biologically active material. The coating of the medical device of the present invention further comprises particles comprising a magnetic material, i.e., magnetic particles . . . ”

One embodiment of the medical device 1001 (see FIG. 16) is illustrated in FIG. 17, which shows a cross-sectional view of a coated strut 1020 of the stent. In the embodiment depicted in FIG. 17, the coated strut 1020 comprises a strut 1025 having a surface 1030. The coated strut 1020 has a composite coating that comprises a first coating layer 1040 that contains a biologically active material 1045; in one embodiment, this first coating layer 1040 also contains polymeric material.

Referring again to FIG. 17, a second coating layer 1050 comprising nanomagnetic particles 1055 is disposed over the first coating layer 1040. This second coating layer 1055, in one embodiment, also includes polymeric material.

Referring again to FIG. 17, and in the preferred embodiment depicted, a third coating layer or sealing layer 1060 is disposed on top of the second coating layer 1050.

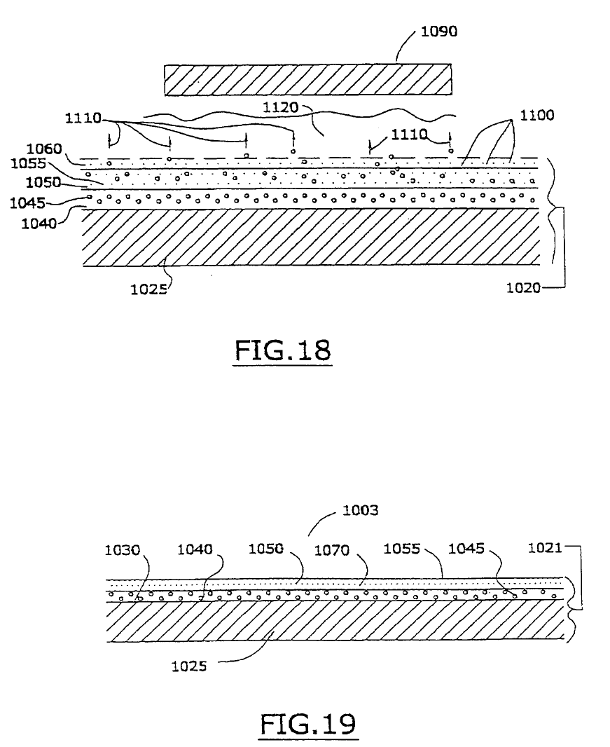

FIG. 18 is similar to FIG. 2B of U.S. published patent application 2004/0030379; and it illustrates the effect of exposing a patient (not shown), who is implanted with a stent having struts 1020 shown in FIG. 17, to an electromagnetic energy source or field 1090. When such a field 1090 is applied, the magnetic particles 1055 move out of the second coating layer 1050 in the direction of upward arrow 1110. This movement disrupts the sealing layer 1160 and forms channels 1100 in such sealing layer 1060.

Referring again to FIG. 18, it will be seen that the size of the channels 1100 formed generally depends on the size of the magnetic particles 1055 used. The biologically active material 1045 can then be released from the coating through the disrupted sealing layer 1060 into the surrounding tissue 1120. The duration of exposure to the field and the strength of the electromagnetic field 1090 determine the rate of delivery of the biologically active material 1045.

FIG. 19 illustrates another coated stent 1003; this Figure is similar to FIG. 3A of U.S. published patent application 2004/0030379. Referring to FIG. 19, and in the preferred embodiment depicted therein, it will be seen that, in this embodiment, the coated strut 1021 contains a coating comprised of a first coating layer 1040 comprising a biologically active material 1045 and preferably a polymeric material disposed over the surface 1030 of the strut 1025. A second coating layer or sealing layer 1070 comprising magnetic particles 1055 and a polymeric material is disposed on top of the first coating layer 1040.

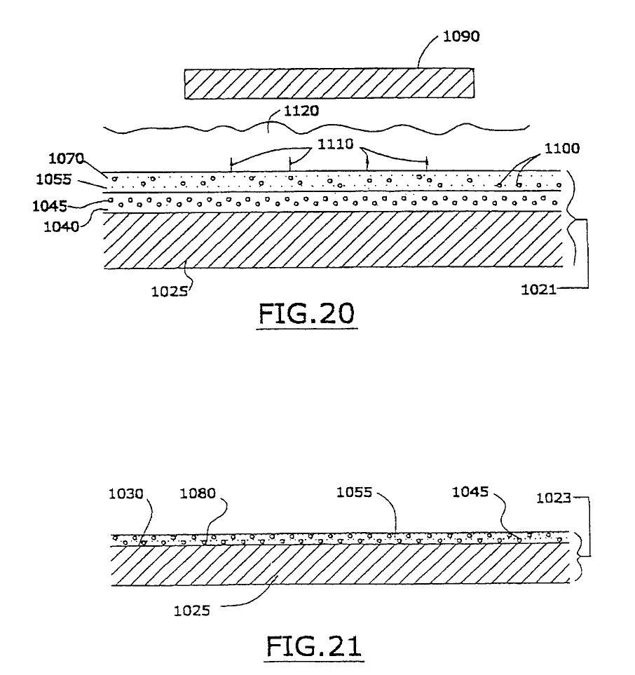

FIG. 20 illustrates the effect of exposing a patient (not shown) who is implanted with a stent having struts 1021 shown in FIG. 19 to an electromagnetic field 1090; this Figure is similar to FIG. 3B of U.S. published patent application 2004/0030379. Referring to FIG. 20 when such a field 1090 is applied, the magnetic particles 1055 move through the sealing layer 1070 as shown by the upward arrow 1110, and they create channels 1100 in the sealing layer 1070. The biologically active material 1045 in the underlying first coating layer 1040 is allowed to travel through the channels 1100 in the sealing layer 1070 and be released to the surrounding tissue 1120. Since the biologically active material 1045 is in a separate first coating layer 1040 and must migrate through the second coating layer or the sealing layer 1070, the release of the biologically active material 1045 is controlled after formation of the channels 1100.

FIG. 21 is similar to FIG. 4A of published U.S. patent application 2004/0030379, and it shows another embodiment of a coated stent strut 1023. In this embodiment, the coating comprises a coating layer 1080 comprising a biologically active material 1045, magnetic particles 1055, and a polymeric material.

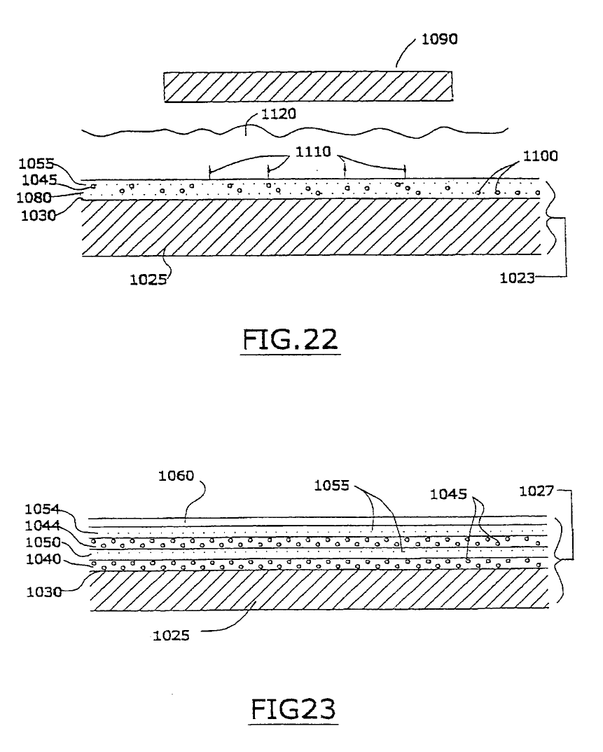

FIG. 22, which is similar to FIG. 4B of published U.S. patent application 2004/0030379, illustrates the effect of exposing a patient (not shown) who is implanted with a stent having struts 1023 to an electromagnetic field 1090. The field 1090 is applied, the magnetic particles 1055 move through the layer 1080 as shown by the arrow 1110 and create channels in the coating layer 1080. The biologically active material 1045 can then be released to the surrounding tissue 1120.

In another embodiment, and referring to FIGS. 16 and 23, the medical device 1001 of the present invention may be a stent having struts coated with a coating comprising more than one coating layer containing a magnetic material. FIG. 23 illustrates such a coated strut 1027. The coating comprises a first coating layer 1040 containing a polymeric material and a biologically active material 1045 which is disposed on the surface 1030 of a strut 1025. A second coating layer 1050 comprising a polymeric material and magnetic particles 1055 is disposed over the first coating layer 1040. A third coating layer 1044 comprising a polymeric material and a biologically active material 1045 is disposed over the second coating layer 1050. A fourth coating layer 1054 comprising a polymeric material and magnetic particles 1055 is disposed over this third layer 1044. Finally a sealing layer 1060 of a polymeric material is disposed over the fourth coating layer 1054. The permeability of the coating layers may be different from layer to layer so that the release of the biologically active material from each layer can differ. Also, the magnetic susceptibility of the magnetic particles may differ from layer to layer. The magnetic susceptibility may be varied using different concentrations or percentages of magnetic particles in the coating layers. The magnetic susceptibility of the magnetic particles may also be varied by changing the size and type of material used for the magnetic particles. When the magnetic susceptibility of the magnetic particles differs from layer to layer, different excitation intensity and/or frequency are required to activate the magnetic particles in each layer.

Referring again to FIG. 23, (and also to paragraph 27 at page 3 of published U.S. patent application 2004/0030379), the nanomagnetic particles preferably used in the embodiment depicted in FIG. 23 may be coated with a biologically active material and then incorporated into a coating for the medical device. In one embodiment, the biologically active material is a nucleic acid molecule. The nucleic acid coated nanomagnetic magnetic particles may be formed by painting, dipping, or spraying the magnetic particles with a solution comprising the nucleic acid. The nucleic acid molecules may adhere to the nanomagnetic particles via adsorption. Also the nucleic acid molecules may be linked to the magnetic particles chemically, via linking agents, covalent bonds, or chemical groups that have affinity for charged molecules. Application of an external electromagnetic field can cause the adhesion between the biologically active material and the magnetic particle to break, thereby allowing for release of the biologically active material.

In another embodiment, and referring to such FIGS. 16-23, the magnetic particles may be molded into or coated onto a non-metallic medical device, including a bio-absorb able medical device. The magnetic properties of the preferred nanomagnetic particles allow the non-metallic implant to be extracorporally imaged, vibrated, or moved. In specific embodiments, the nanomagnetic particles are painted, dipped or sprayed onto the outer surface of the device. The nanomagnetic particles may also be suspended in a curable coating, such as a UV curable epoxy, or they may be electrostatically sprayed onto the medical device and subsequently coated with a UV or heat curable polymeric material.

Additionally, and in some embodiments, the movement of the magnetic particles that occurs when the patient implanted with the coated device is exposed to an external electromagnetic field, releases mechanical energy into the surrounding tissue in which the medical device is implanted and triggers histamine production by the surrounding tissues. The histamine has a protective effect in preventing the formation of scar tissues in the vicinity at which the medical device is implanted.

In one embodiment, the movement of the preferred nanomagnetic particles creates a sufficient amount of heat to kill cells by hyperthermia. This embodiment is described elsewhere in this specification, wherein nanomagnetic particles with specified Curie temperatures that preferentially kill cancer cells when heated are described. In one preferred embodiment, the application of the external electromagnetic field 9090 activates the biologically active material in the coating of the medical device. A biologically active material that may be used in this embodiment may be a thermally sensitive substance that is coupled to nitric oxide, e.g., nitric oxide adducts, which prevent and/or treat adverse effects associated with use of a medical device in a patient, such as restenosis and damaged blood vessel surface. The nitric oxide is attached to a carrier molecule and suspended in the polymer of the coating, but it is only biologically active after a bond breaks, thereby releasing the smaller nitric oxide molecule in the polymer and eluting into the surrounding tissue. Typical nitric oxide adducts include, e.g., nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono- or poly-nitrosylated proteins, particularly polynitrosated albumin or polymers or aggregates thereof. The albumin is preferably human or bovine, including humanized bovine serum albumin. Such nitric oxide adducts are disclosed in U.S. Pat. No. 6,087,479 to Stamler et al., the entire disclosure of which is incorporated herein by reference into this specification.

In one embodiment, the application of the electromagnetic field 1090 effects a chemical change in the polymer coating, thereby allowing for faster release of the biologically active material from the coating.

Paragraphs 32-35 of published U.S. patent application 2004/0030379 are applicable to the devices of the instant invention. They are presented herein in their entireties.

“B. Drug Release Modulation Employing a Mechanical Vibrational Energy Source”

“Another embodiment of the present invention is a system for delivering a biologically active material to a body of a patient that comprises a mechanical vibrational energy source and an insertable medical device comprising a coating containing the biologically active material. The coating can optionally contain magnetic particles. After the device is implanted in a patient, the biologically active material can be delivered to the patient on-demand or when the material is needed by the patient. To deliver the biologically active material, the patient is exposed to an extracorporal or external mechanical vibrational energy source. The mechanical vibrational energy source includes various sources which cause vibration such as sonic or ultrasonic energy. Exposure to such energy source causes disruption in the coating that allows for the biologically active material to be released from the coating and delivered to body tissue.”

“Moreover, in certain embodiments, the biologically active material contained in the coating of the medical device is in a modified form. The modified biologically active material has a chemical moiety bound to the biologically active material. The chemical bond between the moiety and the biologically active material is broken by the mechanical vibrational energy. Since the biologically active material is generally smaller than the modified biologically active material, it is more easily released from the coating. Examples of such modified biologically active materials include the nitric oxide adducts described above.”

“In another embodiment, the coating comprises at least a coating layer containing a polymeric material whose structural properties are changed by mechanical vibrational energy. Such change facilitates release of the biologically active material which is contained in the same coating layer or another coating layer.”

Paragraphs 36, 37, 38, 39, 40, and 41 of published U.S. patent application 2004/0030379 are also applicable to the medical devices of this invention. They are presented below in their entireties.

“C. Materials Suitable for the Invention 1. Suitable Medical Devices”

“The medical devices of the present invention are insertable into the body of a patient. Namely, at least a portion of such medical devices may be temporarily inserted into or semi-permanently or permanently implanted in the body of a patient. Preferably, the medical devices of the present invention comprise a tubular portion which is insertable into the body of a patient. The tubular portion of the medical device need not to be completely cylindrical. For instance, the cross-section of the tubular portion can be any shape, such as rectangle, a triangle, etc., not just a circle.”

“The medical devices suitable for the present invention include, but are not limited to, stents, surgical staples, catheters, such as central venous catheters and arterial catheters, guidewires, balloons, filters (e.g., vena cava filters), cannulas, cardiac pacemaker leads or lead tips, cardiac defibrillator leads or lead tips, implantable vascular access ports, stent grafts, vascular grafts or other grafts, interluminal paving system, intra-aortic balloon pumps, heart valves, cardiovascular sutures, total artificial hearts and ventricular assist pumps.”

“Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

“The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

Paragraphs 42-47 of published U.S. patent application 2004/0030379 describes the magnetic particles used in the device of such application. In applicants' preferred device, the magnetic particles of such device are replaced with certain nanomagnetic particles described elsewhere in this specification These nanomangetic particles preferably have the properties described below.

The nanomagnetic particles are usually in to form of a coating a nanomagnetic material comprised of such particles. An assembly comprised of a device, wherein said device comprises a substrate and, disposed over such substrate, nanomagnetic material and magetoresistive material, wherein the nanomagnetic material has a saturation magnetization of from about 2 to about 3000 electromagnetic units per cubic centimeter. The nanomagnetic particles generally have an average particle size of less than about 100 nanometers, wherein the average coherence length between adjacent nanomagnetic particles is less than 100 nanometers.

In one embodiment, the nanomagnetic material has an average particle size of less than about 20 nanometers and a phase transition temperature of less than about 200 degrees Celsius.

In one embodiment, the average particle size of such nanomagnetic particles is less than about 15 nanometers. In another embodiment, the nanomagentic material has a saturation magnetization of at least 2,000 electromagnetic units per cubic centimeter.

In yet another embodiment, the nanomagnetic material has a saturation magnetization of at least 2,500 electromagnetic units per cubic centimeter.

In yet another embodiment, the nanomagnetic, the particles of nanomagnetic material have a squareness of from about 0.05 to about 1.0.

In yet another embodiment, the nanomagnetic, the particles of nanomagnetic material are at least triatomic, being comprised of a first distinct atom, a second distinct atom, and a third distinct atom. In one aspect of this embodiment, the first distinct atom is an atom selected from the group consisting of atoms of actinium, americium, berkelium, californium, cerium, chromium, cobalt, curium, dysprosium, einsteinium, erbium, europium, fermium, gadolinium, holmium, iron, lanthanum, lawrencium, lutetium, manganese, mendelevium, nickel, neodymium, neptunium, nobelium, plutonium, praseodymium, promethium, protactinium, samarium, terbium, thorium, thulium, uranium, and ytterbium. In another aspect of this embodiment, the distinct atom is a cobalt atom.

In yet another embodiment, the particles of nanomagnetic material are comprised of atoms of cobalt and atoms of iron.

In yet another embodiment, such first distinct atom is a radioactive cobalt atom. In yet another embodiment, the particles of nanomagnetic material are comprised of a said first distinct atom, said second distinct atom, said third distinct atom, and a fourth distinct atom. In one aspect of this embodiment, the particles of nanomagnetic material are comprised of a fifth distinct atom.

In yet another embodiment, such particles of nanomagnetic material have a sqareness of from about 0.1 to about 0.9. In one aspect of this embodiment, such particles of nanomagnetic material have a squareness is from about 0.2 to about 0.8.

In yet another embodiment, the nanomagnetic particles have an average size of less of less than about 3 nanometers. In yet another embodiment, the nanomagnetic particles have an average size of less than about 15 nanometers. In yet another embodiment, the nanomagnetic particles have an average size is less than about 11 nanometers.

In yet another embodiment, the nanomagnetic particles have a phase transition temperature of less than 46 degrees Celsius. In yet another embodiment, the nanomagnetic particles have a phase transition temperature of less than about 50 degrees Celsius.

In yet another embodiment, the nanomagnetic material has a coercive force of from about 0.1 to about 10 Oersteds.

In yet another embodiment, the nanomagnetic particles have a relative magnetic permeability of from about 1.5 to about 2,000.

In yet another embodiment, the nanomagnetic particles have a saturation magnetization of at least 100 electromagnetic units per cubic centimeter. In one aspect of this embodiment, the particles of nanomagnetic material have a saturation magnetization of at least about 200 electromagnetic units (emu) per cubic centimeter. In yet another aspect of this embodiment, the particles of nanomagnetic material have a saturation magnetization of at least about 1,000 electromagnetic units per cubic centimeter.

In yet another embodiment, the nanomagnetic particles have a coercive force of from about 0.01 to about 5,000 Oersteds. In one aspect of this embodiment, such particles of nanomagnetic material have a coercive force of from about 0.01 to about 3,000 Oersteds. In yet another embodiment, the nanomagnetic particles have a relative magnetic permeability of from about 1 to about 500,000. In one aspect of this embodiment, such particles have a relative magnetic permeability of from about 1.5 to about 260,000.

In yet another embodiment, the nanomagnetic particles have a mass density of at least about 0.001 grams per cubic centimeter. In one aspect of this embodiment, such particles of nanomagnetic material have a mass density of at least about 1 gram per cubic centimeter. In another aspect of this embodiment, such particles of nanomagnetic material have a mass density of at least about 3 grams per cubic centimeter. In yet another aspect of this embodiment, such particles of nanomagnetic material have a mass density of at least about 4 grams per cubic centimeter.

In yet another embodiment, the second distinct atom of such nanomagnetic particles has a relative magnetic permeability of about 1.0. In one aspect of this embodiment, such second distinct atom is an atom selected from the group consisting of aluminum, antimony, barium, beryllium, boron, bismuth, calcium, gallium, germanium, gold, indium, lead, magnesium, palladium, platinum, silicon, silver, strontium, tantalum, tin, titanium, tungsten, yttrium, zirconium, magnesium, and zinc.

In yet another embodiment, the nanomagnetic particles are comprised of a third distinct atom that is an atom selected from the group consisting of argon, bromine, carbon, chlorine, fluorine, helium, helium, hydrogen, iodine, krypton, oxygen, neon, nitrogen, phosphorus, sulfur, and xenon. In one aspect of this embodiment, the third distinct atom is nitrogen.

In yet another embodiment, the nanomagnetic particles are represented by the formula AxByCz, wherein A is said first distinct atom, B is said second distinct atom, C is said third distinct atom, and x+y+z is equal to 1. In one aspect of this embodiment, such nanomagnetic particles are comprised of atoms of oxygen. In another aspect of this embodiment, the nanomagnetic particles are comprised of atoms of iron which optionally me be radioactive. In another aspect of this embodiment, such nanomagnetic particles are comprised of atoms of cobalt which, optimally, may be radioactive.

In yet another embodiment, the particles of nanomagnetic material are present in the form of a coating with a thickness of from about 400 to about 2000 nanometers. In one aspect of this embodiment, the coating has a thickness of from about 600 to about 1200 nanometers. In another aspect of this embodiment, the coating has a morphological density of at least about 98 percent, preferably at least about 99 percent, and more preferably at least about 99.5 percent. In another aspect of this embodiment, such coating has an average surface roughness of less than about 100 nanometers, and preferably of less than about 10 nanometers. In another aspect of this embodiment, such coating is biocompatible. In another aspect of this embodiment, such coating is hydrophobic. In yet another aspect of this embodiment, such coating is hydrophilic.

Paragraphs 48, through 72 of published U.S. patent application 2004/0030379 describe biologically active material that may be used in the device of this invention. These paragraphs are presented below in their entireties.

“3. Biologically Active Material”

“The term ‘biologically active material’ encompasses therapeutic agents, such as drugs, and also genetic materials and biological materials. The genetic materials mean DNA or RNA, including, without limitation, of DNA/RNA encoding a useful protein stated below, anti-sense DNA/RNA, intended to be inserted into a human body including viral vectors and non-viral vectors. Examples of DNA suitable for the present invention include DNA encoding . . . anti-sense RNA . . . tRNA or rRNA to replace defective or deficient endogenous molecules . . . angiogenic factors including growth factors, such as acidic and basic fibroblast growth factors, vascular endothelial growth factor, epidermal growth factor, transforming growth factor α and β, platelet-derived endothelial growth factor, plateletderived growth factor, tumor necrosis factor α, hepatocyte growth factor and insulin like growth factor . . . cell cycle inhibitors including CD inhibitors . . . thymidine kinase (“TK”) and other agents useful for interfering with cell proliferation, and . . . the family of bone morphogenic proteins (“BMP's”) as explained below. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).”

“The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. Alternatively or in addition, molecules capable of inducing an upstream or downstream effect of a BMP can be provided. Such molecules include any of the “hedgehog” proteins, or the DNA's encoding them. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.”

“Biologically active material also includes non-genetic therapeutic agents, such as: . . . anti-thrombogenic agents such as heparin, heparin derivatives, urokinase, and PPack (dextrophenylalanine proline arginine chloromethylketone); . . . anti-proliferative agents such as enoxaprin, angiopeptin, or monoclonal antibodies capable of blocking smooth muscle cell proliferation, hirudin, and acetylsalicylic acid, amlodipine and doxazosin; . . . anti-inflammatory agents such as glucocorticoids, betamethasone, dexamethasone, prednisolone, corticosterone, budesonide, estrogen, sulfasalazine, and mesalamine; . . . immunosuppressants such as sirolimus (RAPAMYCIN), tacrolimus, everolimus and dexamethasone, . . . antineoplastic/antiproliferative/anti-miotic agents such as paclitaxel, 5-fluorouracil, cisplatin, vinblastine, vincristine, epothilones, methotrexate, azathioprine, halofuginone, adriamycin, actinomycin and mutamycin; cladribine; endostatin, angiostatin and thymidine kinase inhibitors, and its analogs or derivatives; . . . anesthetic agents such as lidocaine, bupivacaine, and ropivacaine; . . . anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; . . . vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; . . . cholesterol-lowering agents; vasodilating agents; and agents which interfere with endogenous vasoactive mechanisms; . . . anti-oxidants, such as probucol; . . . antibiotic agents, such as penicillin, cefoxitin, oxacillin, tobranycin . . . angiogenic substances, such as acidic and basic fibrobrast growth factors, estrogen including estradiol (E2), estriol (E3) and 17-Beta Estradiol; and . . . drugs for heart failure, such as digoxin, beta-blockers, angiotensin-converting enzyme (ACE) inhibitors including captopril and enalopril.”

“Also, the biologically active materials of the present invention include trans-retinoic acid and nitric oxide adducts. A biologically active material may be encapsulated in micro-capsules by the known methods.”

Paragraphs 73 through 82 of published U.S. patent application 1004/0030379 describe coating compositions that may be used in the device of the instant invention; and they are reproduced in their entireties below.

“4. Coating Compositions . . . The coating compositions suitable for the present invention can be applied by any method to a surface of a medical device to form a coating. Examples of such methods are painting, spraying, dipping, rolling, electrostatic deposition and all modern chemical ways of immobilization of bio-molecules to surfaces.”

“The coating composition used in the present invention may be a solution or a suspension of a polymeric material and/or a biologically active material and/or magnetic particles in an aqueous or organic solvent suitable for the medical device which is known to the skilled artisan. A slurry, wherein the solid portion of the suspension is comparatively large, can also be used as a coating composition for the present invention. Such coating composition may be applied to a surface, and the solvent may be evaporated, and optionally heat or ultraviolet (UV) cured.”

“The solvents used to prepare coating compositions include ones which can dissolve the polymeric material into solution and do not alter or adversely impact the therapeutic properties of the biologically active material employed. For example, useful solvents for silicone include tetrahydrofuran (THF), chloroform, toluene, acetone, isooctane, 1,1,1-trichloroethane, dichloromethane, and mixture thereof.”

“A coating of a medical device of the present invention may consist of various combinations of coating layers. For example, the first layer disposed over the surface of the medical device can contain a polymeric material and a first biologically active material. The second coating layer, that is disposed over the first coating layer, contains magnetic particles and optionally a polymeric material. The second coating layer protects the biologically active material in the first coating layer from exposure during implantation and prior to delivery. Preferably, the second coating layer is substantially free of a biologically active material.”

“Another layer, i.e. sealing layer, which is free of magnetic particles, can be provided over the second coating layer. Further, there may be another coating layer containing a second biologically active material disposed over the second coating layer. The first and second biologically active materials may be identical or different. When the first and second biologically active material are identical, the concentration in each layer may be different. The layer containing the second biologically active material may be covered with yet another coating layer containing magnetic particles. The magnetic particles in two different layers may have an identical or a different average particle size and/or an identical or a different concentration. The average particle size and concentration can be varied to obtain a desired release profile of the biologically active material. In addition, the skilled artisan can choose other combinations of those coating layers.”

“Alternatively, the coating of a medical device of the present invention may comprise a layer containing both a biologically active material and magnetic particles. For example, the first coating layer may contain the biologically active material and magnetic particles, and the second coating layer may contain magnetic particles and be substantially free of a biologically active material. In such embodiment, the average particle size of the magnetic particles in the first coating layer may be different than the average particle size of the magnetic particles in the second coating layer. In addition, the concentration of the magnetic particles in the first coating layer may be different than the concentration of the magnetic particles in the second coating layer. Also, the magnetic susceptibility of the magnetic particles in the first coating layer may be different than the magnetic susceptibility of the magnetic particles in the second coating layer.”

“The polymeric material should be a material that is biocompatible and avoids irritation to body tissue. Examples of the polymeric materials used in the coating composition of the present invention include, but not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate, styrene-isobutylene copolymers and blends and copolymers thereof. Also, other examples of such polymers include polyurethane (BAYHDROL®, etc.) fibrin, collagen and derivatives thereof, polysaccharides such as celluloses, starches, dextrans, alginates and derivatives, hyaluronic acid, and squalene. Further examples of the polymeric materials used in the coating composition of the present invention include other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (etylene-propylene-diene) rubbers, fluorosilicones, polyethylene glycol, polysaccharides, phospholipids, and combinations of the foregoing. Preferred is polyacrylic acid, available as HYDROPLUS® (Boston Scientific Corporation, Natick, Mass.), and described in U.S. Pat. No. 5,091,205, the disclosure of which is hereby incorporated herein by reference. In a most preferred embodiment of the invention, the polymer is a copolymer of polylactic acid and polycaprolactone.”

“More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition adheres better to the surface of the medical device when the device is subjected to forces, stress or mechanical challenge.”

“The amount of the polymeric material present in the coatings can vary based on the application for the medical device. One skilled in the art is aware of how to determine the desired amount and type of polymeric material used in the coating. For example, the polymeric material in the first coating layer may be the same as or different than the polymeric material in the second coating layer. The thickness of the coating is not limited, but generally ranges from about 25 μm to about 0.5 mm. Preferably, the thickness is about 30 μm to 100 μm.”

Paragraphs 84 through 92 of published U.S. patent application 2004/0030379 describes certain energy sources which may be used in conjunction with the medical devices of this invention. These paragraphs are presented below in their entireties.

“5. Electromagnetic Sources . . . An external electromagnetic source or field may be applied to the patient having an implanted coated medical device using any method known to skilled artisan. In the method of the present invention, the electromagnetic field is oscillated. Examples of devices which can be used for applying an electromagnetic field include a magnetic resonance imaging (“MRI”) apparatus. Generally, the magnetic field strength suitable is within the range of about 0.50 to about 5 Tesla (Webber per square meter). The duration of the application may be determined based on various factors including the strength of the magnetic field, the magnetic substance contained in the magnetic particles, the size of the particles, the material and thickness of the coating, the location of the particles within the coating, and desired releasing rate of the biologically active material.”

“In an MRI system, an electromagnetic field is uniformly applied to an object under inspection. At the same time, a gradient magnetic field, superposing the electromagnetic field, is applied to the same. With the application of these electromagnetic fields, the object is applied with a selective excitation pulse of an electromagnetic wave with a resonance frequency which corresponds to the electromagnetic field of a specific atomic nucleus. As a result, a magnetic resonance (MR) is selectively excited. A signal generated is detected as a MR signal. See U.S. Pat. No. 4,115,730 to Mansfield, U.S. Pat. No. 4,297,637 to Crooks et al., and U.S. Pat. No. 4,845,430 to Nakagayashi. For the present invention, among the functions of the MRI apparatus, the function to create an electromagnetic field is useful for the present invention. The implanted medical device of the present can be located as usually done for MRI imaging, and then an electromagnetic field is created by the MRI apparatus to facilitate release of the biologically active material. The duration of the procedure depends on many factors, including the desired releasing rate and the location of the inserted medical device. One skilled in the art can determine the proper cycle of the electromagnetic field, proper intensity of the electromagnetic field, and time to be applied in each specific case based on experiments using an animal as a model.

“In addition, one skilled in the art can determine the excitation source frequency of the electromagnetic energy source. For example, the electromagnetic field can have an excitation source frequency in the range of about 1 Hertz to about 300 kilohertz. Also, the shape of the frequency can be of different types. For example, the frequency can be in the form of a square pulse, ramp, sawtooth, sine, triangle, or complex. Also, each form can have a varying duty cycle.”

“6. Mechanical Vibrational Energy Source . . . . The mechanical vibrational energy source includes various sources which cause vibration such as ultrasound energy. Examples of suitable ultrasound energy are disclosed in U.S. Pat. No. 6,001,069 to Tachibana et al. and U.S. Pat. No. 5,725,494 to Brisken, PCT publications WO00/16704, WO00/18468, WO00/00095, WO00/07508 and WO99/33391, which are all incorporated herein by reference. Strength and duration of the mechanical vibrational energy of the application may be determined based on various factors including the biologically active material contained in the coating, the thickness of the coating, structure of the coating and desired releasing rate of the biologically active material.”

“Various methods and devices may be used in connection with the present invention. For example, U.S. Pat. No. 5,895,356 discloses a probe for transurethrally applying focused ultrasound energy to produce hyperthermal and thermotherapeutic effect in diseased tissue. U.S. Pat. No. 5,873,828 discloses a device having an ultrasonic vibrator with either a microwave or radio frequency probe. U.S. Pat. No. 6,056,735 discloses an ultrasonic treating device having a probe connected to a ultrasonic transducer and a holding means to clamp a tissue. Any of those methods and devices can be adapted for use in the method of the present invention.”

“Ultrasound energy application can be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely to an outer surface of the patient body proximal to the inserted medical device.”

“The duration of the procedure depends on many factors, including the desired releasing rate and the location of the inserted medical device. The procedure may be performed in a surgical suite where the patient can be monitored by imaging equipment. Also, a plurality of probes can be used simultaneously. One skilled in the art can determine the proper cycle of the ultrasound, proper intensity of the ultrasound, and time to be applied in each specific case based on experiments using an animal as a model.”

“In addition, one skilled in the art can determine the excitation source frequency of the mechanical vibrational energy source. For example, the mechanical vibrational energy source can have an excitation source frequency in the range of about 1 Hertz to about 300 kilohertz. Also, the shape of the frequency can be of different types. For example, the frequency can be in the form of a square pulse, ramp, sawtooth, sine, triangle, or complex. Also, each form can have a varying duty cycle.”

Paragraphs 93 through 97 of published U.S. patent application 2004/0030379 describe processes for treating body tissue that may be used in conjunction with the medical device of this invention. These paragraphs are presented below in their entireties.”

“D. Treatment of Body Tissue With the Invention . . . . The present invention provides a method of treatment to reduce or prevent the degree of restenosis or hyperplasia after vascular intervention such as angioplasty, stenting, atherectomy and grafting. All forms of vascular intervention are contemplated by the invention, including, those for treating diseases of the cardiovascular and renal system. Such vascular intervention include, renal angioplasty, percutaneous coronary intervention (PCI), percutaneous transluminal coronary angioplasty (PTCA); carotid percutaneous transluminal angioplasty (PTA); coronary by-pass grafting, angioplasty with stent implantation, peripheral percutaneous transluminal intervention of the iliac, femoral or popliteal arteries, carotid and cranial vessels, surgical intervention using impregnated artificial grafts and the like. Furthermore, the system described in the present invention can be used for treating vessel walls, portal and hepatic veins, esophagus, intestine, ureters, urethra, intracerebrally, lumen, conduits, channels, canals, vessels, cavities, bile ducts, or any other duct or passageway in the human body, either in-born, built in or artificially made. It is understood that the present invention has application for both human and veterinary use.”

“The present invention also provides a method of treatment of diseases and disorders involving cell overproliferation, cell migration, and enlargement. Diseases and disorders involving cell overproliferation that can be treated or prevented include but are not limited to malignancies, premalignant conditions (e.g., hyperplasia, metaplasia, dysplasia), benign tumors, hyperproliferative disorders, benign dysproliferative disorders, etc. that may or may not result from medical intervention. For a review of such disorders, see Fishman et al., 1985, Medicine, 2d Ed., J. B. Lippincott Co., Philadelphia.”

“Whether a particular treatment of the invention is effective to treat restenosis or hyperplasia of a body lumen can be determined by any method known in the art, for example but not limited to, those methods described in this section. The safety and efficiency of the proposed method of treatment of a body lumen may be tested in the course of systematic medical and biological assays on animals, toxicological analyses for acute and systemic toxicity, histological studies and functional examinations, and clinical evaluation of patients having a variety of indications for restenosis or hyperplasia in a body lumen.”

“The efficacy of the method of the present invention may be tested in appropriate animal models, and in human clinical trials, by any method known in the art. For example, the animal or human subject may be evaluated for any indicator of restenosis or hyperplasia in a body lumen that the method of the present invention is intended to treat. The efficacy of the method of the present invention for treatment of restenosis or hyperplasia can be assessed by measuring the size of a body lumen in the animal model or human subject at suitable time intervals before, during, or after treatment. Any change or absence of change in the size of the body lumen can be identified and correlated with the effect of the treatment on the subject. The size of the body lumen can be determined by any method known in the art, for example, but not limited to, angiography, ultrasound, fluoroscopy, magnetic resonance imaging, optical coherence tomography and histology.”

A Medical Preparation for Treating Arthrosis, Arthritis, and Other Diseases

In one embodiment of this invention, a novel medical preparation comprised of applicants' nanomagnetic particles is provided. This preparation is similar to the preparation described in U.S. Pat. No. 6,669,623.

U.S. Pat. No. 6,669,623, the entire disclosure of which is hereby incorporated by reference into this specification, discloses and claims “1. A medical preparation including nanoscalar particles that generate heat when an alternating electromagnetic field is applied, said nanoscalar particles comprising: a core containing iron oxide and an inner shell with groups that are capable of forming cationic groups, wherein the iron oxide concentration is in the range from 0.01 to 50 mg/ml of synovial fluid at a power absorption in the range from 50 to 500 mW/mg of iron and heating to a temperature in the range from 42 to 50° C.; and pharmacologically active species bound to said inner shell selected from the group consisting of thermosensitizers and thermosensitive chemotherapeutics or isotopes thereof; wherein said preparation is used for treating arthrosis, arthritis and rheumatic joint diseases by directly injecting said nanoscalar particles into the synovial fluid, said nanoscalar particles being absorbed by said fluid and transported to the inflamed synovial membrane where they are activated after a predefined period of time by applying said alternating electromagnetic field.”

Applicants' medical preparation is similar to the preparation of U.S. Pat. No. 6,669,623 but differs therefrom in that, instead of an iron oxide core, applicants' preparation is comprised of the nanomagnetic material described elsewhere in this specification.

As is disclosed in column 2 of U.S. Pat. No. 6,669,623, “The invention is based on the concept of using a suspension of nanoscalar particles designed based on the description given in DE 197 26 282 for treating rheumatic joint diseases, said particles comprising, in a first embodiment, a core containing iron oxide, an inner shell that encompasses said core and comprises groups capable of forming cationic groups, and an outer shell made of species comprising neutral and/or anionic groups, and radionuclides and cytotoxic substances bound to said inner shell. These nanoscalar particles may also be one-shelled, i.e. consist just of the core and the inner shell, designed as described above . . . . It has been found that despite the fact that phagocytic activity in the synovial fluid decreases as the patients' age increases, intracellular adsorption of the particles according to the invention in macrophages is increased even in pathologically changed macrophage titers in the joint cavity, and that the inflammatory process is controlled as said particles adhere to actively proliferating cells of the synovia. Due to these effects and the heat generated when applying an alternating electromagnetic field, the radionuclides show increased efficacy as compared to radiosynoviorthesis. Last but not least, success of treatment is increased beyond the additive effect of each component due to binding substances that have a cytotoxic effect when exposed to heat to the particles, as this efficiently combines radiotherapy, thermotherapy, and chemotherapy.”

As is disclosed at columns 2-3 of U.S. Pat. No. 6,669,623, “According to an embodiment that utilizes the invention, a suspension of nanoscalar particles formed by an iron oxide core and two shells, with doxorubicin as a heat-sensitive cytotoxic material and beta emitting radionuclides bound to said particles, is directly injected into the joint cavity to be treated. Depending on phagocytic activity in the synovia, the suspension will stay there without generating heat for a period of time that is determined before the therapy begins. This period can be from 1 hour to 72 hours. In this period, the two-shelled nanoparticles according to the invention are absorbed by the synovial fluid and flow into the inflamed synovial membrane. The therapist then ascertains using magnetic resonance tomography whether the nanoparticles are really deposited in the synovial membrane, the adjacent lymph nodes, and in the healthy tissue. If required, an extravasation to adjacent areas may be performed but this should not be necessary due to the high rate of phagocytosis . . . . Subsequently, the area is exposed to an alternating electromagnetic field with an excitation frequency in the range from 1 kHz and 100 MHz. Its actual value depends on the location of the diseased joint. While hands and arms are treated at higher frequencies, 500 kHz will be sufficient for back pain, the lower joints and the thigh joints. The alternating electromagnetic field brings out the localized heat; at the same time, the radionuclide and the cytotoxic substances (here: doxorubicin) are activated, and success of treatment beyond the added effects of its components is achieved due to the trimodal combinatorial effect of therapies and the differential endocytosis and high rate of phagocytosis of the nano-particles. This means that the synovial membrane shows increased and sustained sclerosing with this treatment as compared to other medical preparations and methods of treating rheumatic diseases . . . . The heat that can be generated by the alternating electromagnetic field applied to the nanoparticles, or, in other words, the duration of applying the alternating electromagnetic field to obtain a specific equilibrium temperature is calculated in advance based on the iron oxide concentration that is typically in the range from 0.01 to 50 mg/ml of synovial fluid and power absorption that is typically in the range from 50 to 500 mW/mg of iron. Then the field strength is reduced to keep the temperature on a predefined level of, for example, 45° C. However, there is a considerable temperature drop from the synovial layer treated to adjacent cartilage and bone tissue so that the cartilage layer and the bone will not be damaged by this heat treatment. The temperature in the cartilage layer is slightly increased as compared to normal physiological conditions (38° C. to 40° C.). The resulting stimulation of osteoblasts improves the reconstitution of degeneratively modified bone borders and cartilage. Repeated applications of the alternating electromagnetic field not only counteract recurring inflammation after the decline of radioactivity but—at an equilibrium temperature in the range from 38 to 40° C.—are also used to stimulate osteoblast division. When applying static magnetic field gradients, the particles can be concentrated in the treated joint (‘magnetic targeting’).” The iron-oxide core of the particles of this U.S. Pat. No. 6,669,223 may advantageously be replaced with the nanomagnetic material core of the present invention.

By way of further illustration, one may replace the iron-oxide containing core of the nanoparticles of published U.S. patent application U.S. 2003/0180370 with the nanomagnetic material of this invention. The entire disclosure of this published United States patent application is hereby incorporated by reference into this specification.

Claim 1 of published U.S. patent application 2003/0180370 describes “1. Nanoscale particles having an iron oxide-containing core and at least two shells surrounding said core, the (innermost) shell adjacent to the core being a coat that features groups capable of forming cationic groups and that is degraded by the human or animal body tissue at such a low rate that an association of the core surrounded by said coat with the surfaces of cells and the incorporation of said core into the inside of cells, respectively is possible, and the outer shell(s) being constituted by species having neutral and/or anionic groups which, from without, make the nanoscale particles appear neutral or negatively charged and which is (are) degraded by the human or animal body tissue to expose the underlying shell(s) at a rate which is higher than that for the innermost shell but still low enough to ensure a sufficient distribution of said nanoscale particles within a body tissue which has been punctually infiltrated therewith.” The particles of this published application comprise an iron-oxide-contianing core with at least two shells (coats).

As is disclosed in paragraphs 0005 and 0006 of published U.S. patent application 2003/018370, “ . . . such particles can be obtained by providing a (preferably superparamagnetic) iron oxide-containing core with at least two shells (coats), the shell adjacent to the core having many positively charged functional groups which permits an easy incorporation of the thus encased iron oxide-containing cores into the inside of the tumor cells, said inner shell additionally being degraded by the (tumor) tissue at such a low rate that the cores encased by said shell have sufficient time to adhere to the cell surface (e.g. through electrostatic interactions between said positively charged groups and negatively charged groups on the cell surface) and to subsequently be incorporated into the inside of the cell. In contrast thereto, the outer shell(s) is (are) constituted by species which shield (mask) or compensate, respectively, or even overcompensate the underlying positively charged groups of the inner shell (e.g. by negatively charged functional groups) so that, from without, the nanoscale particle having said outer shell(s) appears to have an overall neutral or negative charge. Furthermore the outer shell(s) is (are) degraded by the body tissue at a (substantially) higher rate than the innermost shell, said rate being however still low enough to give the particles sufficient time to distribute themselves within the tissue if they are injected punctually into the tissue (e.g. in the form of a magnetic fluid). In the course of the degradation of said outer shell(s) the shell adjacent to the core is exposed gradually. As a result thereof, due the outer shell(s) (and their electroneutrality or negative charge as seen from the exterior) the coated cores initially become well distributed within the tissue and upon their distribution they also will be readily imported into the inside of the tumor cells (and first bound to the surfaces thereof, respectively), due to the innermost shell that has been exposed by the biological degradation of the outer shell(s) . . . . Thus the present invention relates to nanoscale particles having an iron oxide-containing core (which is ferro-, ferri- or, preferably, superparamagnetic) and at least two shells surrounding said core, the (innermost) shell adjacent to the core being a coat that features groups capable of forming cationic groups and that is degraded by the human or animal body tissue at such a low rate that an association of the core surrounded by said coat with the surfaces of cells and the incorporation of said core into the inside of cells, respectively is possible, and the outer shell(s) being constituted by species having neutral and/or anionic groups which, from without, make the nanoscale particles appear neutral or negatively charged and which is (are) degraded by the human or animal body tissue to expose the underlying shell(s) at a rate which is higher than that for the innermost shell but still low enough to ensure a sufficient distribution of said nanoscale particles within a body tissue which has been punctually infiltrated therewith.”

Paragraph 0007 of published United States patent application U.S. 2003/0180370 indicates that the core of the particles of this patent application “ . . . consists of pure iron oxide . . . .” Applicants advantageously substitute their nanomagnetic material of this invention for such “ . . . pure iron oxide . . . .”

The shells of published United States patent application U.S. 2003/0180370 are discussed in paragraphs 0013 through 0016 of such patent application. As is disclosed in these paragraphs, “According to the present invention one or more (preferably one) outer shells are provided on the described innermost shell . . . the outer shell serves to achieve a good distribution within the tumor tissue of the iron oxide-containing cores having said inner shell, said outer shell being required to be biologically degradable (i.e., by the tissue) after having served its purpose to expose the underlying innermost shell, which permits a smooth incorporation into the inside of the cells and an association with the surfaces of the cells, respectively. The outer shell is constituted by species having no positively charged functional groups, but on the contrary having preferably negatively charged functional groups so that, from without, said nanoscale particles appear to have an overall neutral charge (either by virtue of a shielding (masking) of the positive charges inside thereof and/or neutralization thereof by negative charges as may, for example, be provided by carboxylic groups) or even a negative charge (for example due to an excess of negatively charged groups). According to the present invention for said purpose there may be employed, for example, readily (rapidly) biologically degradable polymers featuring groups suitable for coupling to the underlying shell (particularly innermost shell), e.g., (co)polymers based on α-hydroxycarboxylic acids (such as, e.g., polylactic acid, polyglycolic acid and copolymers of said acids) or polyacids (e.g., sebacic acid). The use of optionally modified, naturally occurring substances, particularly biopolymers, is particularly preferred for said purpose. Among the biopolymers the carbohydrates (sugars) and particularly the dextrans may, for example, be cited. In order to generate negatively charged groups in said neutral molecules one may employ, for example, weak oxidants that convert part of the hydroxyl or aldehyde functionalities into (negatively charged) carboxylic groups).”

Published U.S. patent application 2003/0180370 also discloses that: “ . . . in the synthesis of the outer coat one is not limited to carbohydrates or the other species recited above but that on the contrary any other naturally occurring or synthetic substances may be employed as well as long as they satisfy the requirements as to biological degradability (e.g. enzymatically) and charge or masking of charge, respectively . . . The outer layer may be coupled to the inner layer (or an underlying layer, respectively) in a manner known to the person skilled in the art. The coupling may, for example, be of the electrostatic, covalent or coordination type. In the case of covalent interactions there may, for example, be employed the conventional bond-forming reactions of organic chemistry, such as, e.g., ester formation, amide formation and imine formation. It is, for example, possible to react a part of or all of the amino groups of the innermost shell with carboxylic groups or aldehyde groups of corresponding species employed for the synthesis of the outer shell(s), whereby said amino groups are consumed (masked) with formation of (poly-)amides or imines. The biological degradation of the outer shell(s) may then be effected by (e.g., enzymatic) cleavage of said bonds, whereby at the same time said amino groups are regenerated.”

The particles of published U.S. patent application 2003/0180370 (and the related particles of the instant invention) may be used to deliver therapeutic agents to the inside of cells in the manner disclosed in paragraphs 0017 et seq. of published U.S. patent application 2003/0180370. As is disclosed in such published patent application, “Although the essential elements of the nanoscale particles according to the present invention are (i) the iron oxide-containing core, (ii) the inner shell which in its exposed state is positively charged and which is degradable at a lower rate, and (iii) the outer shell which is biologically degradable at a higher rate and which, from without, makes the nanoscale particles appear to have an overall neutral or negative charge, the particles according to the invention still may comprise other, additional components. In this context there may particularly be cited substances which by means of the particles of the present invention are to be imported into the inside of cells (preferably tumor cells) to enhance the effect of the cores excited by an alternating magnetic field therein or to fulfill a function independent thereof. Such substances are coupled to the—inner shell preferably via covalent bonds or electrostatic interactions (preferably prior to the synthesis of the outer shell(s)). This can be effected according to the same mechanisms as in the case of attaching the outer shell to the inner shell. Thus, for example in the case of using aminosilanes as the compounds constituting the inner shell, part of the amino groups present could be employed for attaching such compounds. However, in that case there still must remain a sufficient number of amino groups (after the degradation of the outer shell) to ensure the smooth importation of the iron oxide-containing cores into the inside of the cells. Not more than 10% of the amino groups present should in general be consumed for the importation of other substances into the inside of the cells. However, alternatively or cumulatively it is also possible to employ silanes different from aminosilanes and having different functional groups for the synthesis of the inner shell, to subsequently utilize said different functional groups for the attachment of other substances and/or the outer shell to the inner shell. Examples of other functional groups are, e.g., unsaturated bonds or epoxy groups as they are provided by, for example, silanes having (meth)acrylic groups or epoxy groups.”

Published U.S. patent application 2003/0180370 also discloses that “According to the present invention it is particularly preferred to link to the inner shell substances which become completely effective only at slightly elevated temperatures as generated by the excitation of the iron oxide-containing cores of the particles according to the invention by an alternating magnetic field, such as, e.g., thermosensitive chemotherapeutic agents (cytostatic agents, thermosensitizers such as doxorubicin, proteins, etc.). If for example a thermosensitizer is coupled to the innermost shell (e.g. via amino groups) the corresponding thermosensitizer molecules become reactive only after the degradation of the outer coat (e.g. of dextran) upon generation of heat (by the alternating magnetic field).”

Such “thermosensitive chemotherapeutic agents” are also referred to in claim 18 of U.S. Pat. No. 6,541,039 (“ . . . at least one pharmacologically active species is selected from the group consisting of thermosensitizers and thermosensitive chemotherapeutic agents), and in claim 6 of U.S. Pat. No. 6,669,623 (“thermosensitive cytotxic agents bound to said inner shell); the entire disclosure of each of these United States patent applications is hereby incorporated by reference into this specification.

These “thermosensitive cytotoxic agents” are also referred to in paragraph 18 of published U.S. patent application U.S. 2003/0180370, wherein it is disclosed that: “According to the present invention it is particularly preferred to link to the inner shell substances which become completely effective only at slightly elevated temperatures as generated by the excitation of the iron oxide-containing cores of the particles according to the invention by an alternating magnetic field, such as, e.g., thermosensitive chemotherapeutic agents (cytostatic agents, thermosensitizers such as doxorubicin, proteins, etc.). If for example a thermosensitizer is coupled to the innermost shell (e.g. via amino groups) the corresponding thermosensitizer molecules become reactive only after the degradation of the outer coat (e.g. of dextran) upon generation of heat (by the alternating magnetic field).”

The activity of the compositions of published United States patent application U.S. 2003/0180370 (and of applicants' derivative compositions) is described in paragraphs 0019-0020 of published U.S. patent application 2003/0180370. As is disclosed in these paragraphs, “For achieving optimum results, e.g. in tumor therapy, the excitation frequency of the alternating magnetic field applicator must be tuned to the size of the nanoscale particles according to the present invention in order to achieve a maximum energy yield. Due to the good distribution of the particle suspension within the tumor tissue, spaces of only a few micrometers in length can be bridged in a so-called “bystander” effect known from gene therapy, on the one hand by the generation of heat and on the other hand through the effect of the thermosensitizer, especially if excited several times by the alternating field, with the result that eventually the entire tumor tissue becomes destroyed . . . . Particles leaving the tumor tissue are transported by capillaries and the lymphatic system into the blood stream, and from there into liver and spleen. In said organs the biogenous degradation of the particles down to the cores (usually iron oxide and iron ions, respectively) then takes place, which cores on the one hand become excreted and on the other hand also become resorbed and introduced into the body's iron pool. Thus, if there is a time interval of at least 0.5 to 2 hours between the intralesional application of magnetic fluid and the excitation by the alternating field the surrounding environment of the tumor itself has “purged” itself of the magnetic particles so that during excitation by the alternating field indeed only the lesion, but not the surrounding neighborhood will be heated.”

When, however, the particles in question are nano-sized (as is the case with applicants' nanomagnetic particles), they do not leave the tissue in which they have been applied. Thus, as is disclosed in paragraph 0021 of published U.S. patent application 2003/0180370, “ . . . nanoparticles do not leave the tissue into which they have been applied, but get caught within the interstices of the tissue. They will get transported away only via vessels that have been perforated in the course of the application. High molecular weight substances, on the other hand, leave the tissue already due to diffusion and tumor pressure or become deactivated by biodegradation. Said processes cannot take place with the nanoscale particles of the present invention since on the one hand they are already small enough to be able to penetrate interstices of the tissue (which is not possible with particles in the μm range, for example, liposomes) and on the other hand are larger than molecules and, therefore cannot leave the tissue through diffusion and capillary pressure. Moreover, in the absence of an alternating magnetic field, the nanoscale particles lack osmotic activity and hardly influence the tumor growth, which is absolutely necessary for an optimum distribution of the particles within the tumor tissue . . . . If an early loading of the primary tumor is effected the particles will be incorporated to a high extent by the tumor cells and will later also be transferred to the daughter cells at a probability of 50% via the parental cytoplasm. Thus, if also the more remote surroundings of the tumor and known sites of metastatic spread, respectively are subjected to an alternating magnetic field individual tumor cells far remote from the primary tumor will be affected by the treatment as well. Particularly the therapy of affected lymphatic nodes can thus be conducted more selectively than in the case of chemotherapy. Additional actions by gradients of a static magnetic field at sites of risk of a subsequent application of an alternating field may even increase the number of hits of loaded tumor cells.”

The composition of published United States patent application U.S. 2003/0180370, and also of applicants' related composition, also effect an anti-mitotic activity because of “selective embolization.” Thus, as is disclosed in paragraphs 24-25 of such United States patent application, “Due to the two-stage interlesional application a selective accumulation is not necessary. Instead the exact localization of the lesion determined in the course of routine examination and the subsequently conducted infiltration, in stereotactic manner or by means of navigation systems (robotics), of the magnetic fluid into a target region of any small (or bigger) size are sufficient . . . The combination with a gradient of a static magnetic field permits a regioselective chemoembolization since not only the cyctostatic agent preferably present on the particles of the invention is activated by heat but also a reversible aggregation of the particles and, thus a selective embolization may be caused by the static field.”

It is known that, when cancer cells are treated with hyperthermia, the survival levels of cells treated in the absence of nutrients is greatly reduced over those heat treated with nutrients; see, e.g., an article by G. M. Hahn, “Metabolic aspects of the role of hyperthermia in mammalian cell inactivation and their possible relevance to cancer treatment,” Cancer Res. 34: 3117-3123, November, 1974. In this Hahn article, it was disclosed that “The sensitivity of cells to hyperthermia (as well as their ability to repair heat-induced damage after 43 degrees) is strongly related to their nutritional history. Chinese hamster cells chronically deprived of serum (and probably other medium components) become extremely heat sensitive.

In one embodiment of the instant invention, applicants' “two-shell nanomagnetic compositons” are incorporated into tumor cells and, with the use of an external electromagnetic field, used to cause a regioselective embolization. Thereafter, when the tumor cells have been deprived of serum, the nanomagnetic materials permanently disposed within the cells are caused to heat up and kill the cells, which are now more sensitive to hyperthermia.

Other applications for applicants' compositions (and the related compositions of published U.S. patent application 2003/0180370) are discussed in paragraphs 0026 and 0027 of such patent application, wherein it is disclosed that: “In addition to tumor therapy, further applications of the nanoscale particles according to the present invention (optionally without the outer shell(s)) are the heat-induced lysis of clotted microcapillaries (thrombi) of any localization in areas which are not accessible by surgery and the successive dissolution of thrombi in coronary blood vessels. For example thrombolytic enzymes which show an up to ten-fold increase in activity under the action of heat or even become reactive only on heating, respectively may for said purpose be coupled to the inner shell of the particles according to the invention. Following intraarterial puncture of the vessel in the immediate vicinity of the clogging the particles will automatically be transported to the “point of congestion” (e.g., under MRT control). A fiberoptical temperature probe having a diameter of, e.g., 0.5 mm is introduced angiographically and the temperature is measured in the vicinity of the point of congestion while, again by external application of an alternating magnetic field, a microregional heating and activation of said proteolytic enzymes is caused. In the case of precise application of the magnetic fluid and of MRT control a determination of the temperature can even be dispensed with on principle since the energy absorption to be expected can already be estimated with relatively high accuracy on the basis of the amount of magnetic fluid applied and the known field strength and frequency. The field is reapplied in intervals of about 6 to 8 hours. In the intervals of no excitation the body has the opportunity to partly transport away cell debris until eventually, supported by the body itself, the clogging is removed. Due to the small size of the particles of the invention the migration of said particles through the ventricles of the heart and the blood vessels is uncritical. Eventually the particles again reach liver and spleen via RES.”

Published United States patent application U.S. 2003/0180370 also discloses that: “Apart from classical hyperthermia at temperatures of up to 46/47° C. also a thermoablation can be conducted with the nanoscale particles of the present invention. According to the state of the art mainly interstitial laser systems that are in part also used in surgery are employed for thermoablative purposes. A big disadvantage of said method is the high invasivity of the microcatheter-guided fiberoptical laser provision and the hard to control expansion of the target volume. The nanoparticles according to the present invention can be used for such purposes in a less traumatic way: following MRT-aided accumulation of the particle suspension in the target region, at higher amplitudes of the alternating field also temperatures above 50° C. can homogeneously be generated. Temperature control may, for example, also be effected through an extremely thin fiberoptical probe having a diameter of less than 0.5 mm. The energy absorption as such is non-invasive.”

The compositions described in published United States patent application U.S. 2003/0180370 may be used in the processes described by the claims of U.S. Pat. No. 6,541,039, the entire disclosure of which is hereby incorporated by reference into this specification.

Claim 1 of U.S. Pat. No. 6,541,039 describes: “1. A method of hyperthermic treatment of a region of the body selected from the group consisting of hyperthermic tumor therapy, heat-induced lysis of a thrombus, and thermoablation of a target region, comprising: (a) accumulating in the region of the body a magnetic fluid comprising nanoscale particles suspended in a fluid medium, each particle having an iron oxide-containing core and at least two shells surrounding said core, (I) the innermost shell adjacent to the core being a shell that: (a) is formed from polycondensable silanes comprising at least one aminosilane and comprises groups that are positively charged or positively chargeable, and (b) is degraded by human or animal body tissue at such a low rate that adhesion of the core surrounded by the innermost shell with the surface of a cell through said positively charged or positively chargeable groups of the innermost shell and incorporation of the core into the interior of the cell are possible, and (2) the outer shell or shells comprising at least one species that: (a) is a biologically degradable polymer selected from (co)polymers based on .alpha.-hydroxycarboxylic acids, polyols, polyacids, and carbohydrates optionally modified by carboxylic groups and comprises neutral and/or negatively charged groups so that the nanoscale particle has an overall neutral or negative charge from the outside of the particle, and (b) is degraded by human or animal body tissue to expose the underlying shell or shells at a rate which is higher than that for the innermost shell but is still low enough to ensure a sufficient distribution of a plurality of the nanoscale particles within a body tissue which has been infiltrated therewith; and (b) applying an alternating magnetic field to generate heat in the region by excitation of the iron oxide-containing cores of the particles, thereby causing the hyperthermic treatment”.

Claims 2-15 of U.S. Pat. No. 6,541,039 are dependent upon claim 1. Claim 3 describes “3. The method of claim 1 that is a method of heat-induced lysis of a thrombus, comprising accumulating in the thrombus the magnetic fluid, and applying an alternating magnetic field to generate heat by excitation of the iron oxide-containing cores of the particles to cause heat-induced lysis of the thrombus.” claim 4 describes “4. The method of claim 1 that is a method of thermoablation of a target region, comprising accumulating in the target region the magnetic fluid, and applying an alternating magnetic field to generate heat by excitation of the iron oxide-containing cores of the particles to cause thermoablation of the target region.” claim 10 describes “10. The method of claim 1 where the innermost shell is derived from aminosilanes.” claim 11 describes “11. The method of claim 1 where the at least one species comprising the outer shell or shells is selected from carbohydrates optionally modified by carboxylic groups.” claim 12 describes “12. The method of claim 11 where the at least one species comprising the outer shell or shells is selected from dextrans optionally modified by carboxylic groups.” claim 13 describes “13. The method of claim 12 where the at least one species comprising the outer shell or shells is selected from dextrans modified by carboxylic groups.” claim 14 describes “4. The method of claim 1 where at least one pharmacologically active species is linked to the innermost shell.” claim 15 describes “15. The method of claim 14 where the at least one pharmacologically active species is selected from the group consisting of thermosensitizers and thermosensitive chemotherapeutic agents.

The other independent claim in U.S. Pat. No. 6,541,039 is claim 16, which describes “16. A method of tumor therapy by hyperthermia, comprising: (a) accumulating in the tumor a magnetic fluid comprising nanoscale particles suspended in a fluid medium, each particle having a superparamagnetic iron oxide-containing core having an average particle size of 3 to 30 nm comprising magnetite, maghemite, or stoichiometric intermediate forms thereof and at least two shells surrounding said core, (1) the innermost shell adjacent to the core being a shell that: (a) is formed from polycondensable aminosilanes and comprises groups that are positively charged or positively chargeable, and (b) is degraded by human or animal body tissue at such a low rate that adhesion of the core surrounded by the innermost shell with the surface of a cell through said positively charged or positively chargeable groups of the innermost shell and incorporation of the core into the interior of the cell are possible, and (2) the outer shell or shells being a shell or shells comprising at least one species that: (a) is a biologically degradable polymer selected from dextrans optionally modified by carboxylic groups and comprises neutral and/or negatively charged groups so that the nanoscale particle has an overall neutral or negative charge from the outside of the particle, and (b) is degraded by human or animal body tissue to expose the underlying shell or shells at a rate which is higher than that for the innermost shell but is still low enough to ensure a sufficient distribution of a plurality of the nanoscale particles within a body tissue which has been infiltrated therewith; and (b) applying an alternating magnetic field to generate heat in the tumor by excitation of the iron oxide-contain cores of the particles, thereby causing hyperthermia of the tumor.”

Claims 17 and 18 of U.S. Pat. No. 6,541,039 are dependent upon claim 16. Claim 17 describes “17. The method of claim 16 where at least one pharmacologically active species is linked to the innermost shell.” claim 18 describes “18. The method of claim 17 where the at least one pharmacologically active species is selected from the group consisting of thermosensitizers and thermosensitive chemotherapeutic agents.”

As will be apparent to those skilled in the art, all of the processes described in U.S. Pat. No. 6,541,039 may be conducted with a composition that contains applicants' nanomagnetic material rather than the iron oxide material of the Lesniak et al. patent.

The nanosize iron-containing oxide particles used in the process of U.S. Pat. No. 6,541,039 may be prepared by conventional means such as, e.g., the process described in U.S. Pat. No. 6,183,658. This latter patent claims “1. A process for producing an-agglomerate-free suspension of stably coated nanosize iron-containing oxide particles, comprising the following steps in the order indicated: (1) preparing an aqueous suspension of nanosize iron-containing oxide particles which are partly or completely present in the form of agglomerates; (2) adding (i) a trialkoxysilane which has a hydrocarbon group which is directly bound to Si and to which is bound at least one group selected from amino, carboxyl, epoxy, mercapto, cyano, hydroxy, acrylic, and methacrylic, and (ii) a water-miscible polar organic solvent whose boiling point is at least 10° C. above that of water; (3) treating the resulting suspension with ultrasound until at least 70% of the particles present have a size within the range from 20% below to 20% above the mean particle diameter; (4) removing the water by distillation under the action of ultrasound; and (5) removing the agglomerates which have not been broken up.”

An Anticancer Agent Releasing Microcapsule

In one embodiment of the invention, a microcapsule for hyperthermia treatment is made by coating nanomagnetic particles with cis-platinum diamine dichloride (CDDP), and then covering the layer of anticancer agent with a mixture of hydroxylpropyl cellulse and mannitol. This microcapsule is similar to the microcapsule described in an article by Tomoya Sato et al., “The Development of Anticancer Agent Releasing Microcapusle Made of Ferromagnetic Amorphous Flakes for Intratissue Hyperthermia,” IEEE Transactions on Magnetics, Volume 29, Noumber 6, November, 1993.

The “core” of the Sato et al. microcapsule was ferromagnetic amorphous flakes with an average size of about 50 microns and a Curie temperature of about 45 degrees Centigrade. In one embodiment of the instant invention, the Sato et al. ferromagnetic material is replaced with the nanomagnetic material of this invention.

The core of the Sato et al. microcapsule was then coated with an anticancer agent, such as Cis-platinum diammine dichloride (CDDP). Thereafter, the coated cores were then coated with a material that did not react with the anticancer agent. As is disclosed on page 3329 of the article, “A wide variety of anticancer agents and macromolecular compounds can be used for coating of amorphous flakes, but the absence of reaction between the anticancer agent and the macromolecular compound as the base is the primary condition for their selection. In this study, CDDP was used as the anticancer agent, and a mixture of hydroxypropyl cellulse (HPC-H) and mannitol, which do not react with CDDP, was used as the macromolecular coating material.”

The coating used in the Sato et al. microcapsule was designed to dissolve in bodily fluid when it was heated to a temperature greater than about 40 degrees Centigrade. Thus, as is disclosed at page 3329 of the Sato et al. article, “We noted the characteristics of HPC-H that it becomes a viscous gel in water at 38 degrees C. or below but loses its viscosity above 40 degrees C. Because of this property, we expected that it would remain a viscous gel and slowly release CDDP at body temperatures of 36 to 37 degrees C. but would lose its viscosity and release more CDDP when it is heated to 40 degrees C. or above, and we attempted to regulate the release of CDDP by hyperthermia.”

A Stent that can be Visualized by Magnetic Resonance Imaging

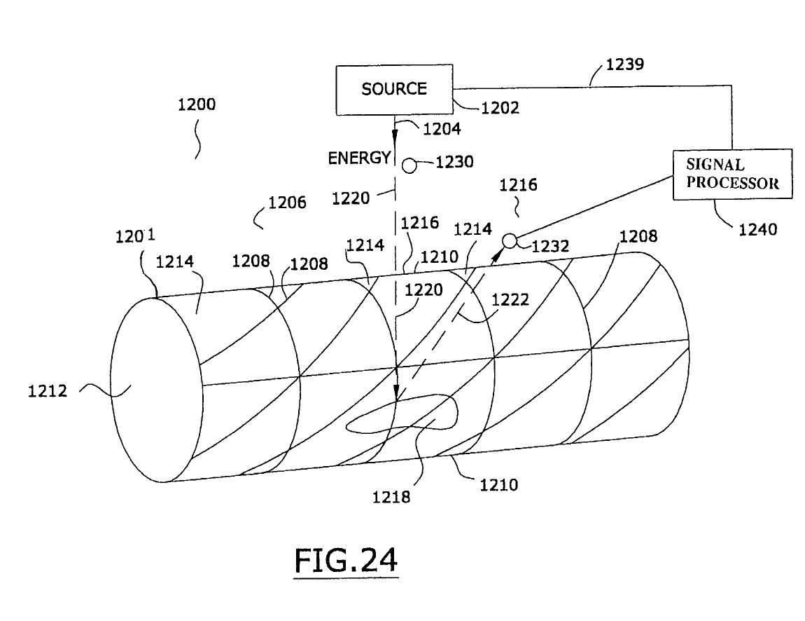

FIG. 24 is a schematic illustration of a stent assembly 1200 that can be readily visualized by magnetic resonance imaging. The stent assembly 1200 preferably contains a metallic stent 1201.

As used in this specification, the term “metallic stent” refers to a stent that is comprised of at least about 80 weight percent of metallic material and, preferably, at least about 90 weight percent of metallic material. Reference may be had, e.g., to U.S. Pat. Nos. 5,562,922; 5,665,103; 5,830,179 (urological stent therapy system); U.S. Pat. No. 5,843,172 (porous medicated stent); U.S. Pat. Nos. 6,027,811; 6,159,237 (implantable vascular and endoluminal stents); U.S. Pat. Nos. 6,174,305; 6,187,054; 6,238,421 (method for metallic implants in living beings); U.S. Pat. No. 6,403,635 (method of treating atherosclerosis or restenosis using microtubule stabilizing agent); U.S. Pat. No. 6,468,300 (stent covering heterologous tissue); U.S. Pat. No. 6,569,104 (Ni—Ti—W alloy); U.S. Pat. Nos. 6,605,109; 6,626,940; 6,679,980 (apparatus for electropolishing a stent); U.S. Pat. No. 6,712,844 (MRI compatible stent); U.S. Pat. Nos. 6,730,120; 6,753,071; 6,776,795 (Ni—Ti—W alloy), and the like. The entire disclosure of each of these United States patents is hereby incorporated by reference into this specification.

Metallic materials are described, e.g., at pages 522-523 of George S. Brady et al.'s “Materials Handbook,” Thirteenth Edition (McGraw-Hill, Inc., New York, N.Y., 1991). As is disclosed in this text, “About three-quarters of the elements available can be classified as metals . . . . Although the word metal, by strict definition, is limited to the pure metal elements, common usage gives it wider scope to include metal alloys. While pure metallic elements have a broad range of properties, they are quite limited in commercial use. Metal alloys, which are combinations of two or more elements, are far more versatile and for this reason are the form in which most metals are used by industry.”

As is also disclosed in the Brady et al. text, “Metallic materials are crystalline solids. Individual crystals are composed of unit cells repeated in a regular pattern to form a three-dimensional crystal lattice structure. A piece of metal is an aggregate of many thousands of interlocking crystals (grains) immersed in a cloud of negative valence electrons detached from the crystals' atoms. These loose electrons serve to hold the crystal structures together because of their electrostatic attraction to the positively charged metal atoms (ions). The bonding forces, being large because of the close-packed nature of metallic crystal structures, account for the generally good mechanical properties of metals. Also, the electron cloud makes most metals good conductors of heat and electricity.”

The Brady et al. work also discloses that “There are two families of metallic materials—ferrous and non-ferrous. The basic ingredient of all ferrous metals is the element iron. These metals range from cast irons and carbon steels, with over 90% iron, to specialty iron alloys, containing a variety of other elements that add up to nearly half the total composition.”

Several metallic stents are described in Patrick W. Serruys et al.'s “Handbook of Coronary Stents,” Fourth Edition (Martin Dunitz Ltd., London, England, 2002). These metallic stents may comprise stainless steel (ARTHOS stent), 316L stainless steel (ANTARES STARFLEX stent), 316L stainless steel coated with phosphorylcholine (BIODIVYSIO stent), 316 LVM stainless steel (SIRIUS stent), 316 L medical grade stainless steel coated with DYLYN (DYLYN stent), 316 stainless steel, polytetrafluoroethylene (JOSTENT stent), Nitinol (JOSTENT BIFLEX stent), niobium alloy coated with indium oxide (LUNAR stent), 316 LVM stainless steel (NEXUS stent), stainless steel plated with gold (NIROYAL stent), 316L stainless steel coated with hypothombogenenic a-SiC:H (RITHRON stent), and the like.

Referring to FIG. 24, and in the preferred embodiment depicted therein, it will be seen that stent assembly 1200 is comprised of a source 1202 of energy 1204.

In one preferred embodiment, the energy 1204 is energy typically emitted by a magnetic resonance imaging (MRI) apparatus and comprises both a static magnetic field with an MRI field strength of from about 0.1 Tesla to about 30 Tesla, a gradient magnetic field of from about 1 to about 200 kilohertz, and an alternating current electromagnetic field with a frequency of from about 1 megahertz to about 3 terahertz.

In one embodiment, the static magnetic field has a field strength of from about 0.5 Tesla to about 20 Tesla. In another embodiment, the static magnetic field has a field strength of from about 1 Tesla to about 10 Tesla. In yet another embodiment, the static magnetic field has a field strength of from about 1.5 Tesla to about 3.5 Tesla.

In one embodiment, the energy 1204 is comprised of an input alternating current electromagnetic field with a frequency of from about 1 megahertz to about 2 gigahertz and, more preferably, from about 50 megahertz to about 1 gigahertz. In one aspect of this embodiment, the input alternating current electromagnetic field has a frequency of from about 50 megahertz to about 300 megahertz.

Referring again to FIG. 24, and in the preferred embodiment depicted therein, a stent 1206 is comprised of a multiplicity of struts 1208 that define an exterior surface 1210 and an interior cavity 1212. A multiplicity of openings 1214 are defined are also defined by such struts; and these openings 1214 facilitate communication between the interior cavity 1212 and the areas 1216 disposed outside of such exterior surface 1210.

In the embodiment depicted in FIG. 24, biological material 1218 is disposed within the stent lumen 1212. In the prior art devices, this biological material would be screened from the energy 1204; and whatever energy did reach the interior area of the stent would not be retransmitted through such outer surface 1210.

Thus, and referring again to U.S. Pat. No. 6,712,844 (the entire disclosure of which is hereby incorporated by reference into this specification), “Because stents are constructed of electrically conductive materials, they suffer from a Faraday Cage effect when used with MRI's. Generically, a Faraday Cage is a box, cage, or array of electrically conductive material intended to shield its contents from electromagnetic radiation. The effectiveness of a Faraday Cage depends on the wave length of the radiation, the size of the mesh in the cage, the conductivity of the cage material, its thickness, and other variables. Stents do act as Faraday Cages in that they screen the stent lumen from the incident RF pulses of the MRI scanner. This prevents the proton spins of water molecules in the stent lumen from being flipped or excited.” Thus, and referring again to FIG. 24, in the prior art stent assemblies the input energy 1204 (and especially the input radio frequency energy) is substantially screened” . . . from the incident RF pulses of the MRI scanner . . . ”; and very little, if any, of such incident RF pulses 1220 penetrate past the outer surface 1210 of the stent to reach the inner lumen 1212 and the biological material 1218.

To the extent that such incident RF pulses 1220 do penetrate the outer surface 1210 of the stent, they will interact with the biological material 1218 to produce an output signal 1222. This output signal 1222 generally does not have a fixed phase relationship with the input signal 1220 in the prior art stent assemblies. Thus, as is also disclosed in U.S. Pat. No. 6,712,844, “The stent's high magnetic susceptibility, however, perturbs the magnetic field in the vicinity of the implant. This alters the resonance condition of protons in the vicinity, thus leading to intravoxel dephasing with an attendant loss of signal” (see column 2 of such patent). This phenomenon of intravoxel dephasing is also discussed in U.S. Pat. No. 5,283,526 (method for performing single and multiple slice magnetic resonance spectroscopic imaging), U.S. Pat. No. 6,069,949 (gradient characterization using Fourier-transform), U.S. Pat. No. 6,408,201 (method and apparatus for efficient stenosis identification in peripheral arterial vasculature using MR imaging), U.S. Pat. No. 6,472,872 (real-time shimming of polarizing field in magnetic resonance system), U.S. Pat. No. 6,587,708 (method for coherent steady-state imaging of constant-velocity flowing fluids), U.S. Pat. No. 6,618,607 (MRI imaging methods using a single excitation), and the like. Reference also may be had, e.g., to published United States patent applications U.S.20020041833A1 (method of magnetic resonance imaging), U.S.20020082497 (MRI imaging methods using a single excitation), and U.S.20020188345A1 (MRI compatible stent). The entire disclosure of each of these United States patents, and of each of these published United States patent applications, is hereby incorporated by reference into this specification.

Referring again to FIG. 24, and in the preferred embodiment depicted therein, in the prior art stent assemblies the output signal 1222 has a difficult time in escaping the exterior surface 1210 of the stent. Thus, and referring again to U.S. Pat. No. 6,712,844 (see column 2), “ . . . the stent Faraday Cage likely impedes the escape of whatever signal is generated in the lumen. The stent's high magnetic susceptibility, however, perturbs the magnetic field in the vicinity of the implant. This alters the resonance condition of protons in the vicinity, thus leading to intravoxel dephasing with an attendant loss of signal. The net result with current metallic stents, most of which are stainless steel, is a signal void in the MRI images. Other metallic stents, such as those made from Nitinol, also have considerable signal loss in the stent lumen due to a combination of Faraday Cage and magnetic susceptibility effects.”

In applicants' stent assembly 1200, by comparison, the output signal 1222 is not “dephased,” i.e., it has a fixed phase relationship with the input signal 1220. The term “fixed phase relationship” is well known to those skilled in the art. Reference may be had, e.g., to U.S. Pat. Nos. 3,581,011; 3,594,738; 3,611,127; 3,611,144; 3,659,942; 3,669,209; 3,691,475; 3,774,115; 3,777,691; 3,784,930; 3,792,473; 3,851,247; 3,921,087; 3,932,811; 4,035,833; 4,038,756; 4,118,125; 4,142,489; 4,152,703; 4,164,577; 4,188,573; 4,204,151; 4,392,020; 4,499,534; 4,642,675; 4,700,359; 4,842,477; 4,872,164; 4,877,974; 4,914,421; 4,924,420; 4,965,810; 4,989,219; 5,315,232; 5,333,074; 5,337,040; 5,345,240; 5,528,112; 5,586,042; 5,722,744; 5,872,959; 6,047,808; 6,278,334; 6,348,826; 6,553,835; 6,583,645; and the like. The entire disclosure of each of these United States patents is hereby incorporated by reference into this specification.

Referring again to FIG. 24, and in the preferred embodiment depicted therein, the input alternating current electromagnetic field 1220 may be represented by the formula Acos (2πft+φ₀), wherein A is the magnitude of the input alternating current electromagnetic field (and is preferably from about 1×10⁻⁶ Tesla to about 100×10⁻⁶ Tesla), f is the frequency of the input alternating current electromagnetic field (and preferably is from about 1 megahertz to about 2 gigahertz), and φ₀ is the initial phase of the input alternating current electromagnetic field 1220 when t is 0 seconds.

By comparison, and referring again to FIG. 24, and in the preferred embodiment depicted therein, the output alternating current electromagnetic field 1222 may be represented by the formula Bcos (2πft+φ₁), wherein B is the magnitude of the output alternating current electromagnetic field 1222, f is the frequency of the output alternating current electromagnetic field, and φ₁ is the phase of the output alternating current electromagnetic field 1222 when φ₁ is measured in relation to t₀.

A fixed phase relationship exists between the input signal 1220 and the output signal 1222 when the following equation is satisfied: φ₁−φ₀=±C±2πn, wherein φ₁ is the phase of the output signal 1222, φ₀ is the phase of the input signal 1220, C is a number between 0 and 360 degrees, and n is an integer including 0.

Referring again to FIG. 24, and to the preferred embodiment depicted therein, it will be seen that implantable magnetic field detectors 1230 and 1232 may be used to detect input signal 1220 and output signal 1222. As will be apparent, one may also refer to the calibration of source 1202 to determine the characteristics of input signal 1230.

In one preferred embodiment, not shown, the magnetic field detectors 1230 and 1232 are omitted and external sources of radiation and detection are used in place of such omitted detectors 1230/1232. In one aspect of this embodiment, a set of coils is used to emit and receive radio frequency energy. In one aspect of this embodiment, such coils are phased array coils that are used to measure the energy 1204 that is supplied to the stent assembly, the energy that penetrates the stent assembly, and the energy that is retransmitted by the stent assembly.

In one embodiment, such set of coils are phased array coils. These coils, are their uses, are well known in the MRI art. Reference may be had, e.g., to U.S. Pat. No. 4,985,678 (horizontal field iron core magnetic resonance scanner), U.S. Pat. No. 5,394,087 (multiple quadrature surface coil system for simultaneous imaging in magnetic resonance imaging), U.S. Pat. No. 5,521,056 (orthogonal adjustment of magnetic resonance surface coils), U.S. Pat. No. 5,578,925 (vertical field quadrature phased array coil system), U.S. Pat. No. 6,097,186 (phased array coil, receive signal processing circuit, and MRI apparatus), U.S. Pat. No. 6,177,795 (spectral component imaging using phased array coils), U.S. Pat. No. 6,396,273 (magnetic resonance imaging receiver/transmitter coils), U.S. Pat. No. 6,411,090 (magnetic resonance imaging transmit coil), U.S. Pat. No. 6,469,406 (autocorrection of MR images acquired using phased array coils), U.S. Pat. No. 6,492,814 (self localizing receive coils for MR), U.S. Pat. No. 6,534,983 (multi-channel phased array coils having minimum mutual inductance for magnetic resonance systems), U.S. Pat. No. 6,604,697 (magnetic resonance imaging receiver/transmitter coils), U.S. Pat. No. 6,608,480 (RF coil for homogeneous quadrature transmit and multiple channel receive), U.S. Pat. No. 6,639,406 (apparatus for decoupling quadrature phased array coils), U.S. Pat. No. 6,714,013 (magnetic resonance imaging receiver/transmitter coils), U.S. Pat. No. 6,724,923 (automatic coil selection of multi-receiver MR data using fast prescan data analysis), U.S. Pat. No. 6,738,501 (adaptive data differentiation and selection from multi-coil receiver to reduce artifacts in reconstruction), U.S. Pat. No. 6,747,452 (decoupling circuit for magnetic resonance imaging local coils), U.S. Pat. No. 6,762,606 (retracting MRI head coil), U.S. Pat. No. 6,781,379 (cable routing and potential equalizing ring for magnetic resonance imaging coils), U.S. Pat. No. 6,788,057 (open architecture gradient coil set for magnetic resonance imaging apparatus), and the like. The entire disclosure of each of these United States patents is hereby incorporated by reference into this specification.

Referring again to FIG. 24, and to the embodiment depicted therein, the probes 1230 and 1232 may be conventional magnetic field detectors. One may use, e.g., conventional magnetic field detectors such as, e.g., the magnetic field detectors disclosed in U.S. Pat. No. 3,829,883 (magnetic field detector employing plural drain IGFET), U.S. Pat. No. 3,835,377 (three terminal magnetoresistive magnetic field detector), U.S. Pat. No. 4,064,453 (magnetic field detector), U.S. Pat. No. 4,210,083 (alternating magnetic field detector), U.S. Pat. Nos. 4,218,975, 4,714,880 (wide frequency pass band magnetic field detector), U.S. Pat. Nos. 4,767,989, 4,875,785, 5,187,437, 5,194,808 (magnetic field detector using a superconductor magnetoresistive element), U.S. Pat. No. 5,309,096 (magnetic field detector for a medical device implantable in the body of patient), U.S. Pat. No. 5,309,097 (video display terminal magnetic field detector), U.S. Pat. No. 5,317,251 (peak magnetic field detector with non-volatile storage), U.S. Pat. Nos. 5,365,391, 5,389,880 (hall analog magnetic field detector), U.S. Pat. No. 5,424,642 (magnetic field detector with a resiliently mounted electrical coil), U.S. Pat. No. 5,517,112 (magnetic field detector with noise blanking), U.S. Pat. No. 5,521,500 (thin-film magnetic field detector), U.S. Pat. No. 5,598,273 (highly sensitive magnetic field detector using low noise DC SQUID), U.S. Pat. No. 5,619,137 (chopped low power magnetic field detector with hysteresis memory), U.S. Pat. Nos. 5,662,694, 5,709,225 (combined magnetic field detector and activity detector employing a capacitive sensor for a medical implant), U.S. Pat. No. 6,005,383 (electrical current sensor with magnetic field detector), U.S. Pat. No. 6,144,196 (magnetic field measuring apparatus and apparatus for measuring spatial resolution of magnetic field detector), U.S. Pat. No. 6,396,264 (shielded loop magnetic field detector), U.S. Pat. Nos. 6,683,397, 6,750,648 (magnetic field detector having a dielectric looped face), and the like. The entire disclosure of each of these United States patents is hereby incorporated by reference into this specification.

In one preferred embodiment, each of the magnetic field detectors 1230/1232 is an implantable medical field detector such as, e.g., the “medical field detector and telemetry unit for implants” described and claimed in U.S. Pat. No. 5,545,187, the entire disclosure of which is hereby incorporated by reference into this specification. Claim 1 of this patent describes” 1. A combination magnetic field detector and threshold unit for use in a medical implant, comprising: a telemetry circuit connected to a voltage source; control logic which generates control signals respectively for telemetry and magnetic field detection; a coil unit including a plurality of coil unit parts; switch means, controlled by said control logic for, when said control logic generates a control signal for telemetry, electrically connecting said coil unit into said telemetry circuit for forming means for receiving and transmitting telemetry signals and for, when said control logic generates a control signal for magnetic field detection, electrically connecting said coil unit parts for forming a primary side and a secondary side of a pulse transformer which generates an output signal having a characteristic which varies dependent on the presence of a magnetic field; and magnetic field indicator means, connected to said secondary side of said pulse transformer, for generating a signal indicating the presence of a magnetic field when said characteristic satisfies a predetermined condition.”

U.S. Pat. No. 5,545,187 contains an excellent discussion of some “prior art” magnetic field sensors. It discloses that “In a medical implant, such as a pacemaker, a magnetic field detector is used for non-invasive activation of different functions in the implant in combination with a permanent magnet placed in the vicinity of the implant at the outside of the patient's body. Some of the functions which can be activated in, e.g., a pacemaker are: disabling the pacemaker's demand function so the pacemaker adapts its operation to battery capacity and having the pacemaker operate in a special, temporary stimulation mode, e.g., in the case of tachycardia, and in conjunction with pacemaker programming . . . . Outside the implant art, the detection of magnetic fields in a number of different ways, e.g., with the aid of reed switches, by changing the resonance frequency or inductance, etc., is generally known.”

U.S. Pat. No. 5,545,187 also discloses that “One device for determining the strength of a magnetic field is described in an article by Lennart Grahm, “Elektrisk matteknik, Analoga instrument och matmetoder,” part 2, 1977, Elektrisk matteknik, Lund, pp. 543-545. As described therein, the voltage induced in a small test body made of ferromagnetic metal is examined with a Forster probe. The Forster probe consists of a small test body made of a ferromagnetic material with high permeability and provided with two windings, one of which is used for alternating current magnetization and the other is used for measuring the ensuing induced voltage. The larger the constant magnetic field, the greater the amplitude of even harmonics when the probe is placed in a constant magnetic field. Thus, a phase detector with a reference voltage equal to twice the frequency of the excitation current can be used for supplying a signal which increases with an increase in the constant magnetic field.”

U.S. Pat. No. 5,545,187 also discloses that “In the implant art, a conventional magnetic field detector consists of a reed switch. Reed switches, however, are sensitive and rather expensive components which also occupy a relatively large amount of space in the implant . . . In order to eliminate the need for a reed switch, therefore, recent proposals have suggested utilization of the implant's telemetry unit so that the unit can also be used for detecting the presence of a magnetic field, in addition to its telemetry function. U.S. Pat. No. 4,541,431 discloses one such proposal with a combined telemetry and magnetic field detector unit. This unit contains a conventional resonant circuit containing, e.g., a coil used in telemetry for transmitting and receiving data. The resonant circuit is also used for sensing the presence of a magnetic field whose strength exceeds a predefined value. The resonant frequency for the resonant circuit varies with the strength of the magnetic field. The resonant circuit is periodically activated, and the number of zero crossings of its signal with a sensing window with a predefined duration is determined. If a predetermined number of zero crossings occurs, this means that the strength of the magnetic field exceeds the predefined value.”

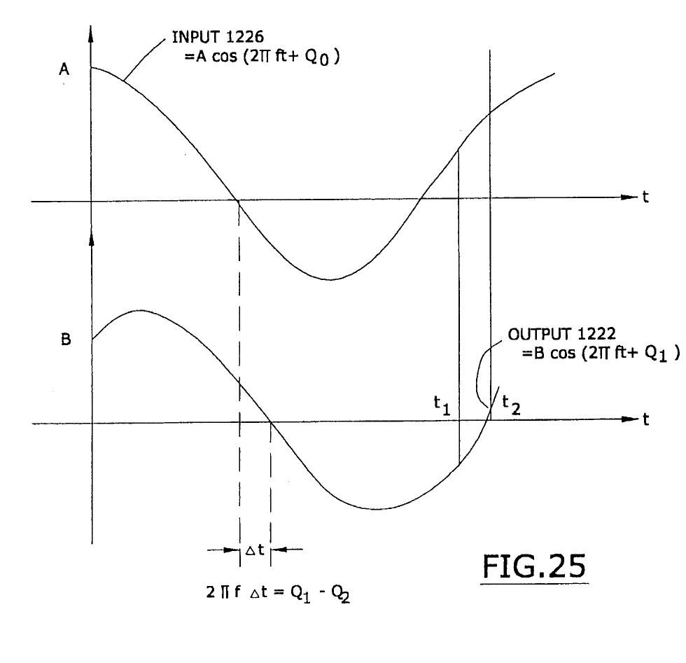

Referring again to FIG. 24, and in one preferred embodiment, the output from probe 1232 may be fed to a signal processor 1240 which, in addition, may also contain information about the input from source 1202. The signal processor 1240 may then be connected to a display (not shown) adapted to display graphs of the input field 1220 and the output field 1222, as illustrated in FIG. 25. From this display, one may determine the magnitude A of the input signal 1220, the magnitude B of the output signal 1222, and the difference in the phases (φ's) of the input and output signals.

As indicated elsewhere in this specification, it is preferred that the input signal 1220 and the output signal 122 have a fixed phase relationship. Furthermore, it is preferred that the ratio of B/A is at least 0.01 and, more preferably, at least about 0.1. In one embodiment, the ratio of B/A is at least 0.2. In yet another embodiment, the ratio of B/A is at least 0.3.

One Preferred Coated Stent Assembly

FIG. 26 is a sectional schematic view, not drawn to scale, of a section of the stent assembly 1200 (see FIG. 24) and, in particular, of a coated strut assembly 1300. Referring to FIG. 26, and in the preferred embodiment depicted therein, it will be seen that each of struts 1208 (see FIG. 24) is preferably coated with a first coating 1312 of nanomagnetic material.

In one preferred embodiment, the coating 1312 has a thickness of at least about 100 nanometers and, more preferably, at least about 500 nanometers. In one aspect of this embodiment, the thickness of coating 1312 is from about 800 nanometers to about 1200 nanometers.

In one preferred embodiment, the nanomagnetic coating 1312 has a magnetization, at a field strength of 2 Tesla, of less than about 100 electromagnetic units (emu) per cubic centimeter and, more preferably, of less than about 10 electromagnetic units per cubic centimeter. In one embodiment, the nanomagnetic coating 1312 has a magnetization, at a field strength of 2 Tesla, of less than about 1 electromagnetic units per cubic centimeters.

In one preferred embodiment, the nanomagnetic coating 1312 has a saturation magnetization of greater than about 1.5 Tesla and, more preferably, of greater than about 1.6 Tesla. In another embodiment, the saturation magnetization of the nanomagnetic coating 1312 is greater than about 2.0 Tesla. In another embodiment, the saturation magnetization of the nanomagnetic coating is greater than about 3.0 Tesla. Put another way, the nanomagnetic coating 1312 does preferably does not reach saturation magnetization at a field strength of 1.5 Tesla, or 1.6 Tesla, or 2.0 Tesla, or 3.0 Tesla, depending upon the embodiment in question.

As is discussed elsewhere in this specification, the nanomagnetic coating 1312 is comprised of nanomagnetic particles that, in one preferred embodiment, have an average particle size of from about 2 to about 100 nanometers and, preferably, from about 3 to about 10 nanometers.

In one embodiment, the nanomagnetic coating 1312 has a resistivity, at a temperature of 300 degrees Kelvin, of from about 1×10⁻² to 1×10⁻⁷ ohm-meters and, preferably, from about 8×10⁻⁵ to about 8×10⁻⁷ ohm-meters.

Referring again to FIG. 26, and in the preferred embodiment depicted therein, a coating 1314 of conductive material is preferably disposed above and contiguous with the coating 1312 of nanomagnetic material. The conductive coating 1314 preferably has a resistivity at a temperature of 300 degrees Kelvin of less than 10⁻⁷ ohm-meters. In one aspect of this embodiment, the conductive coating 1314 preferably has a resistivity of from about 1×10⁻⁸ to about 5×10⁻⁸ ohm-meters. Aluminum is one conductive material that may be used; copper is another conductive material that may be used; and other suitable conductive materials will be apparent to those skilled in the art.

The conductive coating 1314 preferably has a thickness of less than about 100 nanometers and, more preferably, less than about 60 nanometers. In one embodiment, the conductive coating 1314 has a thickness of from about 40 to about 55 nanometers.

Referring again to FIG. 26, and in the preferred embodiment depicted therein, disposed over coating 1314, and contiguous therewith, is dielectric coating 1316. Dielectric coating 1316, which preferably has a thickness of less than about 100 nanometers, also preferably has a dielectric constant larger than 1.0 and, more preferably, larger than 2.0. In one embodiment, the dielectric constant of coating 1316 is preferably greater than 3.0. The values of dielectric constant described are those measured at a temperature of 300 degrees Kelvin.

As is known to those skilled in the art, the dielectric constant for an isotropic medium is the ratio of the capacitance of a capacitor filled with a given dielectric to that of the same capacitor having only a vacuum as dielectric. See, e.g., page 531 of Sybil P. Parker's “McGraw-Hill Dictionary of Scientific and Technical Terms,” Fourth Edition (McGraw-Hill Book Company, New York, N.Y., 1989).

Referring again to FIG. 26, and in the embodiment depicted, disposed on top of dielectric layer 1316 is another coating 1318 of coating material. Conductive layer 1318 preferably has thickness and resistivity properties that are similar to the thickness and resistivity properties of conductive layer 1314.

The conductive layer 1318/dielectric layer 1316/conductive layer 1314 assembly form a capacitor 1322 that, exhibits capacitive reactance in the presence of a radio frequency field. The nanomagentic layer 1312 enclosing the strut 1310 forms an inductor that exhibits inductive reactance in the presence of a radio frequency field. In one embodiment, the dielectric material used is chosen so that, in combination with the inductor assembly, one is near resonance at the frequency of the applied field.

The coatings illustrated in FIG. 26 act as a filter, with a specified inductive reactance and capacitive reactance, that presents minimal impedance to certain frequencies and maximum impedance to other frequencies. In order to “tune the bandwidth” and to allow a reasonable range of frequencies to pass through the filter around the resonant frequency, a resistive layer 1320 is deposited on top of the conductive layer 1318. In one embodiment, the resistive layer 1320 has a thickness less than about 100 nanometers and a resistivity of from about 1×10⁻² to 1×10⁻⁷ ohm-meters.

The construct illustrated in FIG. 26 is merely illustrative of many constructs that may be used to construct filter circuits utilizing strut 1208 and nanomagnetic coating 1312. In one embodiment, a combination of such conductor coatings 1314/1318 and dielectric coatings 1316 are used to construct other circuits.

In one preferred embodiment, one or more cancellation circuits are constructed so that the currents induced by the radio frequency field are out of phase with each other and tend to cancel each other. These (and other) cancellation circuits are well known to those skilled in the art. Reference may be had, e.g., to U.S. Pat. No. 3,720,941 (automatic monopulse clutter cancellation circuit); U.S. Pat. No. 3,715,488 (noise cancellation circuit); U.S. Pat. No. 3,932,713 (induction cancellation circuit); U.S. Pat. Nos. 3,947,848; 4,078,156 (drift cancellation circuit); U.S. Pat. No. 4,204,219 (noise cancellation circuit); U.S. Pat. No. 4,211,978 (cross-talk component cancellation circuit); U.S. Pat. No. 4,214,129 (sideband cancellation circuit); U.S. Pat. No. 4,245,202 (current cancellation circuit); U.S. Pat. No. 4,254,436 (noise cancellation circuit); U.S. Pat. No. 4,268,727 (echo cancellation circuit); U.S. Pat. No. 4,285,006 (ghost cancellation circuit); U.S. Pat. No. 4,341,990 (line ripple cancellation circuit); U.S. Pat. Nos. 4,525,683; 4,528,676 (echo cancellation circuit); U.S. Pat. No. 4,585,987 (sense current cancellation circuit); U.S. Pat. No. 4,629,996 (difference signal distortion cancellation circuit); U.S. Pat. No. 4,688,044 (multiple range interval clutter cancellation circuit); U.S. Pat. No. 4,827,161 (offset voltage cancellation circuit); U.S. Pat. No. 4,932,085 (pilot cancellation circuit); U.S. Pat. No. 5,001,773 (local oscillator feedthru cancellation circuit); U.S. Pat. No. 5,043,814 (adaptive ghost cancellation circuit); U.S. Pat. No. 5,046,133 (interference cancellation circuit); U.S. Pat. No. 5,051,704 (feedforward distortion cancellation circuit); U.S. Pat. No. 5,066,891 (magnetic field cancellation circuit); U.S. Pat. No. 5,161,017 (ghost cancellation circuit); U.S. Pat. No. 5,168,256 (distortion canceling circuit for audio peak limiting); U.S. Pat. No. 5,182,476 (offset cancellation circuit); U.S. Pat. No. 5,428,314 (odd/even order distortion generator and distortion cancellation circuit); U.S. Pat. No. 5,434,446 (parasitic capacitance cancellation circuit); U.S. Pat. No. 5,440,353 (display monitor including moiré cancellation circuit); U.S. Pat. No. 5,561,288 (biasing voltage cancellation circuit); U.S. Pat. No. 5,563,587 (current cancellation circuit); U.S. Pat. No. 5,600,251 (induction noise cancellation circuit); U.S. Pat. No. 5,659,588 (filter leakage cancellation circuit); U.S. Pat. No. 5,719,907 (phase jitter cancellation circuit); U.S. Pat. No. 5,793,551 (differential input capacitance cancellation circuit); U.S. Pat. No. 5,796,301 (offset cancellation circuit); U.S. Pat. No. 5,929,692 (ripple cancellation circuit); U.S. Pat. No. 5,977,892 (offset cancellation circuit); U.S. Pat. No. 6,052,422 (analog signal offset cancellation circuit); U.S. Pat. No. 6,167,247 (leak cancellation circuit); U.S. Pat. No. 6,172,564 (intermodulation product cancellation circuit); U.S. Pat. No. 6,208,135 (inductive noise cancellation circuit); U.S. Pat. No. 6,211,724 (glitch cancellation circuit); U.S. Pat. No. 6,243,430 (noise cancellation circuit); U.S. Pat. No. 6,281,889 (Moiré cancellation circuit); U.S. Pat. No. 6,333,947 (interference cancellation system); U.S. Pat. No. 6,344,756 (echo cancellation circuit); U.S. Pat. No. 6,429,749 (cancellation circuit that suppresses electromagnetic interference); U.S. Pat. No. 6,496,064 (intermodulation product cancellation circuit); U.S. Pat. No. 6,549,054 (DC offset cancellation circuit); U.S. Pat. No. 6,566,934 (charge cancellation circuit); U.S. Pat. No. 6,671,075 (offset voltage cancellation circuit); U.S. Pat. No. 6,693,805 (ripple cancellation circuit); U.S. Pat. No. 6,792,056 (cancellation circuit that suppresses electromagnetic interference using a function generator); and the like. The entire disclosure of each of these United States patents is hereby incorporated by reference into this specification. SEARCH NOTCH filter, also bandPASS FILTER.

Coated strut 1208 assemblies, such as assembly 1300, may be constructed so as to include one or more of the cancellation circuits described in the patents in the prior paragraph of this specification. Such circuits may be constructed by using conductive and/or dielectric coatings. Alternatively, or additionally, one or more components of such circuits may be printed on the surface(s) of one or more of such coatings by conventional means.

FIG. 27 is a sectional view of another preferred coated strut assembly 1400 that differs from the strut assembly 1300 in that, disposed about strut 1208, is a first coating 1312 of nanomagnetic material, a second coating 1316 of dielectric material, a third coating 1314 of conductive material, a fourth coating 1313 of nanomagnetic material (which may be the same as or different than coating 1312), a fifth coating 1317 of dielectric material (which may be the same as or different than coating 1316), and a sixth coating 1318 of conductive material (which may be the same as or different than coating 1314). The combination of coatings 1402 (which includes coatings 1314/1316/1312) is believed to form an equivalent circuit 1436 (see FIG. 28). The combination of coatings 1404 (which includes coatings 1313/1317/1318) is believed to form an equivalent circuit 1438.

Without wishing to be bound to any particular theory or theories, applicants believe that the circuit depicted in FIG. 28 is a reasonably accurate depiction of the equivalent circuit that exists in assembly 1400.

The strut 1208 contains both some resistance 1426 and inductance 1408 and inductance 1409. When strut 1208 is subjected to a radiofrequency field 1410 produced by the radio frequency generator of an MRI machine (not shown), a capacitance 1411 in series with inductance 1408 forms a series resonant circuit 1412 that preferably has a net reactance of zero at the frequency of the radiofrequency (which generally is either 64 megahertz or 128 megahertz, corresponding to d.c. field strengths of 1.5 Tesla and 3.0 Tesla, respectively).

The equivalent resistance 1426 is the resistive loss in the circuit caused by ohmic loss in the various coatings. This equivalent resistance 1426 is used in a well known manner to adjust the bandwidth of the series resonant circuit. The equation for a series resonant frequency is 1/(LC)^{0.5}. The equation for the bandwidth of such a circuit is R/L.

Referring again to FIG. 28, and in the preferred equivalent circuit depicted therein, there is another parallel resonant circuit 1414 comprised of inductance 1409 and capacitance 1413. The inductance 1409 comes from the inductive coatings that often contain nanomagnetic material; it also comes, in part, from the conductive substrate. The capacitance 1413 comes from the configuration of a dielectric coating between conductive materials; it also may come form interconnections (via vias) between various coating layers, as will be described in more detail later in this specification.

The resonant frequency of the parallel circuit 1414 is given by the equation 1/(LC)^{0.5}. As will be apparent, in the parallel circuit configuration, the inductance is contributed by inductor 1409, and the capacitance is contributed by capacitor 1413.

At this parallel resonant frequency, the impedance is substantially infinite; and the input 1410 is thus coupled to the load 1415. The equivalent load 1415 is the interior of the metallic stent 1201 (see FIG. 24).

As will be apparent to those skilled in the art, and referring again to FIG. 27, modification of one or more of the coatings 1312, 1313, 1314, 1316, 1317, and/or 1318 will simultaneously modify both the values of the resistance, inductance, and capacitance presented by such coatings, and will also simultaneously modify the impedance of such coatings.

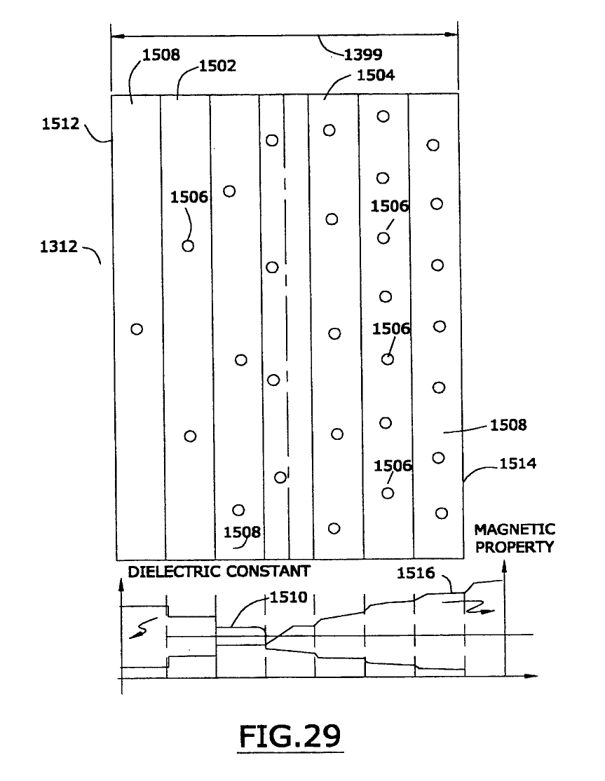

FIG. 29 is a schematic illustration of one preferred nanomagnetic coating 1312 that preferably has a thickness 1399 of from about 800 to about 1,200 nanometers and is comprised of a top half 1502 and a bottom half 1504. In one aspect of this embodiment, at least 60 weight percent of magnetic particles 1506 are disposed in the bottom half 1504 of the coating 1312.

In the embodiment depicted in FIG. 29, the magnetic particles 1506 are disposed within a dielectric matrix 1508. Inasmuch as at least 60 weight percent of the magnetic particles 1506 are disposed in the bottom half 1504 of the coating 1312, at least about 55 weight percent of the dielectric material is disposed in the top half 1502 of the coating 1312.

Without wishing to be bound to any particular theory, applicants believe that this non-homogeneous distribution of the magnetic “A moiety” (and its compounds) is due to the fact that the “A moiety” (which, in one preferred embodiment, is iron) often has a higher atomic weight than the “B” moiety (which, in one preferred embodiment, is aluminum).

Thus, in the embodiment depicted, a plot 1510 of the dielectric constant of the coating 1312 indicates that it decreases as one goes from the top 1512 of coating 1312 to its bottom 1514. Conversely, a plot 1516 of the magnetic properties of the coating 1312 indicates that it increases as one goes from the top 1512 of coating 1312 to its bottom 1514.

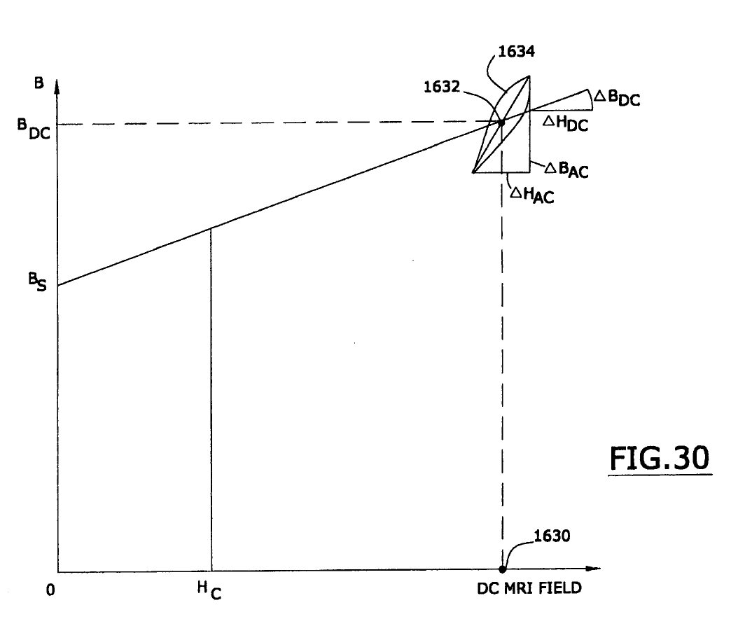

FIG. 30 is a graph of the magnetization curve for coating 1312 (see FIG. 28) in which B (the magnetic flux density, in centimeter-gram-second units) is plotted versus H (the applied field, in Tesla). In the graph depicted in FIG. 30, Hc represents the coercive force, and Bs represents the saturation magnetic flux density, and these parameters help define major hysteresis loop.

The H value at point 1630 is of particular interest. This is the d.c. field strength that is generally present in a magnetic resonance imaging (MRI) field, as it usually is either 1.5 Tesla or 3.0 Tesla. As is known to those skilled in the art, an M.R.I. d.c. field strength of 1.5 Tesla is often associated with an alternating current electromagnetic field with a frequency of 64 megahertz, and an MRI d.c. field strength of 3.0 Tesla is often associated with an alternating current electromagnetic field with a frequency of 128 megahertz.

In the preferred embodiment depicted in FIG. 30, at such point 1630 (regardless of whether it is either 1.5 Tesla or 3.0 Tesla), the B/H plot at point 1632 will have a specified d.c. slope; this slope is also often referred to as the “d.c. permeability.” This slope is equal to ΔB_{DC}/ΔH_{DC} at such point 1632, and it preferably is at least 1.1. As will be apparent, for ease of illustration, FIG. 30 is not drawn to scale.

In one preferred embodiment, the d.c. slope of the B/H plot at a d.c. field strength of either 1.5 Tesla or 3.0 Tesla is at least about 1.2 and, more preferably, at least 1.3. In another embodiment, such slope is at least 1.5.

Referring again to FIG. 30, at such point 1630 (be it either 1.5 Tesla or 3.0 Tesla), the coating 1312 will have a magnetization of less than about 100 electromagnetic units per cubic centimeter (emu/cm³) and, more preferably, less than about 10 emu/cm³. In one preferred embodiment, the coating 1312, at such point 1430 (be it either 1.5 Tesla or 3.0 Tesla), has a magnetization of less than about 5 emu/cm³. In another embodiment, the coating 1312 at such point 1420 has a magnetization of less than about 1 emu/cm³.

Without wishing to be bound to any particular theory, applicants believe that coatings that have large magnetizations at such point 1430 (in excess, e.g., of 1000 emu/cm³) often create undesirable d.c. susceptibility or permeability image artifacts during MRI imaging. It is also believed that coatings that contain in excess of 50 weight percent of an “A moiety” (by combined weight of “A moiety” and “B moiety”) also often create undesirable image artifacts. With regard to FeAlN compositions, applicants have found that when the Fe/[Fe⁺ Al] ratio is 0.9, or 0.95 to produce a coating, substantial d.c. susceptibility or permeability image artifacts are produced during MRI imaging with such a coating. Equivalently, the BDC value at such point 1632 is too high. The corresponding d.c. magnetization value often exceeds 100 emu/cc.

It is unexpected that coatings that contain less than about 50 weight percent of magnetic material should function well in applicants' invention. This is especially so because the prior art discloses that a bulk composition containing iron and aluminum with at least 30 mole percent of aluminum (by total moles of iron and aluminum) is substantially non-magnetic.

U.S. Pat. No. 6,765,144, the entire disclosure of which is hereby incorporated by reference into this specification, discloses that “Iron containing magnetic materials, such as FeAl, FeAlN and FeAlO, have been fabricated by various techniques. The magnetic properties of those materials vary with stoichiometric ratios, particle sizes, and fabrication conditions; see, e.g., R. S. Tebble and D. J. Craik, “Magnetic Materials”, pp. 81-88, Wiley-Interscience, New York, 1969 As is disclosed in this reference, when the iron molar ratio in bulk FeAl materials is less than 70 percent or so, the materials will no longer exhibit magnetic properties” (see lines 59-67 of Column 37). A similar disclosure appears at lines 6-14 of Column 37 of such patent, wherein it is disclosed that “The molar ratio between iron and aluminum used in this aspect is approximately 70/30. Thus, the starting composition in this aspect is almost non-magnetic. See, e.g., page 83 (FIG. 3.1aii) of R. S. Tebble et al.'s “Magnetic Materials” (Wiley-Interscience, New York, N.Y., 1969); this Figure discloses that a bulk composition containing iron and aluminum with at least 30 mole percent of aluminum (by total moles of iron and aluminum) is substantially non-magnetic.” It should be noted that 70 molar percent of iron is equivalent to about 82.5 weight percent of iron.

In one preferred embodiment, applicant's nanomagnetic material contains both iron and aluminum, wherein the weight/weight ratio of Fe/[Fe+Al] is less than 0.5. In one aspect of this embodiment, such weight/weight ratio is from about 0.05 to about 0.4 and, more preferably, from about 0.05 to about 0.3. In another embodiment, such weight/weight ratio is from about 0.05 to about 0.2.

Referring again to FIG. 30, it will be seen that the B.H graph contains a “minor loop” due to the presence of the alternating current electromagnetic field; this a.c. minor loop is the response of the magnetic material under excitation of the alternating current field. When the direct current field is 1.5 Tesla, the alternating current electromagnetic field has a frequency of 64 megahertz. When the direct current field is 3.0 Tesla, the alternating current electromagnetic field has a frequency of 128 Tesla.

The “alternating current minor loop” is, in general, a well-known phenomenon. Reference may be had, e.g., to U.S. Pat. No. 5,811,965 (“DC and AC current sensor having a minor-loop operated current transformer”); the entire disclosure of this United States patent is hereby incorporated by reference into this specification. Although the concept of an a.c. minor loop is known, to the best of applicants' information and belief, no one has studied such a.c. minor loops at frequencies of at least 64 megahertz under static d.c. fields of at least 1.5 Tesla.

Referring again to FIG. 30, it will be seen that the minor loop 1634 also has a slope at point 1632, defined by ΔB_{AC}/ΔH_{AC}. In one embodiment, this AC minor loop slope at point 1632 is greater than the d.c. slope at such point 1632. In another embodiment, this AC minor loop slope at point 1632 is the same as the d.c. slope at such point 1632. In yet another embodiment, the AC minor loop slope at point 1632 is less than the d.c. slope at such point 1632.

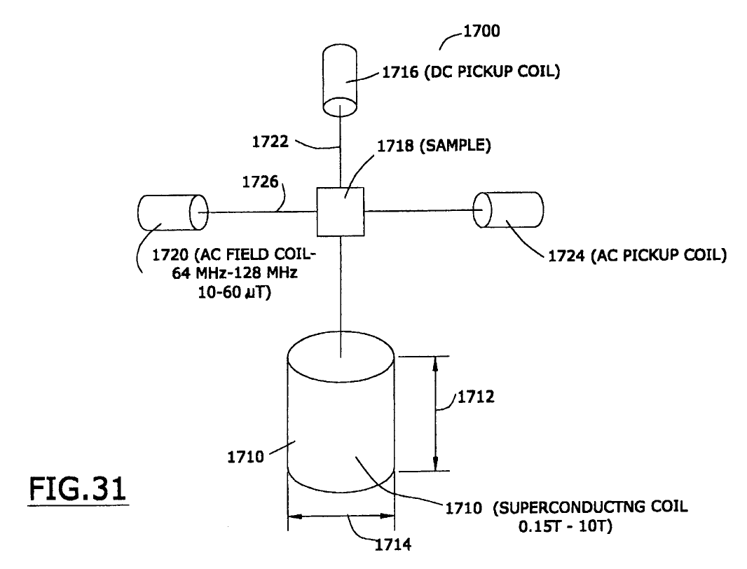

FIG. 31 is a schematic illustration of how one can measure the B/H response at point 1632 to measure both the d.c. slope at such point 1632 and the AC minor loop slope at such point 1632.

One may measure the magnetic properties of a material. including its B/H response, with a magnetometer. As is known to those skilled in the art, a magnetometer is an instrument for measuring the magnitude and sometimes also the direction of a magnetic field. Reference may be had, e.g., to U.S. Pat. No. 3,562,638 (thin film magnetometer using magnetic vector rotation), U.S. Pat. No. 3,622,873 (thin magnetic film magnetometer for providing independent responses from two orthogonal axes), U.S. Pat. No. 3,628,132 (thin magnetic film magnetometer with zero-field reference), U.S. Pat. No. 3,629,697 (paramagnetic resonance and optical pumping magnetometer in the near zero magnetic field range), U.S. Pat. No. 3,731,752 (magnetic detection and magnetometer system therefore), U.S. Pat. No. 3,735,246 (spin coupling nuclear magnetic resonance magnetometer utilizing the same coil for excitation and signal pick-up and using toroidal samples), U.S. Pat. No. 3,781,664 (magnetic detection for an anti-shoplifting system ultilizing combined magnetometer and gradiometer signals), U.S. Pat. No. 3,818,322 (airborn magnetic survey system using two optical magnetometers alternately switched to align with the field during the survey), U.S. Pat. No. 4,437,064 (apparatus for detecting a magnetic anomaly contiguous to remote location by squid gradiometer and magnetometer systems), U.S. Pat. No. 4,506,221 (magnetic heading transducer having dual-axis magnetometer with electromagnetic mounted to permit pivotal vibration thereof), U.S. Pat. No. 4,516,073 (magnetometer probe using a thin-film magnetic material as a magneto-optic sensor), U.S. Pat. No. 4,517,515 (magnetometer with a solid-state magnetic field sensing means), U.S. Pat. No. 4,600,885 (fiber optic magnetometer for detecting DC magnetic fields), U.S. Pat. No. 4,623,842 (magnetometer array with magnetic field sensors on elongate support), U.S. Pat. No. 4,675,606 (magnetometers for detecting metallic objects in earth's magnetic fields), U.S. Pat. No. 4,697,146 (spherical shell fibre optic magnetic field sensors and magnetometers and magnetic field gradients incorporating them), U.S. Pat. No. 4,712,065 (optical fiber magnetometers), U.S. Pat. No. 4,717,873 (magnetic displacement transducer system having a magnet that is movable in a tube whose interior is exposed to a fluid and having at least one magnetometer outside the tube), U.S. Pat. No. 4,728,888 (magnetometer with time coded output of measured magnetic fields), U.S. Pat. No. 4,769,599 (magnetometer with magnetostrictive member of stress variable magnetic permeability), U.S. Pat. No. 4,80,882 (thin film SQUID magnetometer for a device measuring weak magnetic fields), U.S. Pat. No. 4,845,434 (magnetometer circuitry for use in bore hole detection of AC magnetic fields), U.S. Pat. No. 4,864,237 (measuring device having a squid magnetometer with a modulator for measuring magnetic fields of extremely low frequency), U.S. Pat. No. 4,891,592 (nuclear magnetic resonance magnetometer), U.S. Pat. No. 4,937,525 (SQUID magnetometer for measuring weak magnetic fields with gradiometer loops and Josephson tunnel elements on a common carrier), U.S. Pat. No. 4,980,644 (earthquake detecting magnetometer), U.S. Pat. No. 4,996,479 (magnetometer for measuring the magnetic moment of a specimen), U.S. Pat. No. 5,015,953 (magnetometer for detecting DC magnetic field variations), U.S. Pat. No. 5,091,697 (low power, high accuracy magnetometer), U.S. Pat. No. 5,122,744 (gradiometer having a magnetometer that cancels background magnetic field form other magnetometer), U.S. Pat. Nos. 5,126,666, 5,166,614 (integrated-type SQUID magnetometer having a magnetic shield and a multichannel SQUID magnetometer), U.S. Pat. No. 5,184,072 (apparatus for measuring weak static magnetic field using superconduction strips and a SQUID magnetometer, U.S. Pat. Nos. 5,243,281, 5,245,280 (magnetic resonance magnetometer with multiplexed exciting windings), U.S. Pat. No. 5,287,059 (saturable core magnetometer), U.S. Pat. No. 5,291,135 (weak magnetic field measuring system using dc-SQUID magnetometer), U.S. Pat. No. 5,309,095 (compact magnetometer), U.S. Pat. No. 5,444,372 (magnetometer), U.S. Pat. No. 5,525,907 (active impulse magnetometer with bipolar magnetic impulse generator and fast fourier transform receiver to detect sub-surface metallic materials), U.S. Pat. No. 5,530,348 (magnetometer for detecting the intensity of a present magnetic field), U.S. Pat. No. 5,578,926 (locating system for finding magnetic objects in the ground), U.S. Pat. Nos. 5,654,635, 5,684,396 (localizing magnetic dipoles using spatial and temporal processing of magnetometer data), U.S. Pat. No. 5,952,826 (radical solution for nuclear magnetic resonance magnetometer), U.S. Pat. No. 6,313,628 (scalar magnetometer), U.S. Pat. No. 6,496,005 (magnetometer for detecting a magnetic field associated with nuclear magnetic spins or electron spins), U.S. Pat. No. 6,541,967 (fluxgate magnetometer), and the like. The entire disclosure of each of these United States patents is hereby incorporated by reference into this specification.

In one preferred embodiment, the magnetometer used has a superconducting element that allows one to reach a field strength of either 1.5 Tesla and/or 3.0 Tesla. These magnetometers are known to those skilled in the art. Reference may be had to U.S. Pat. No. 3,924,176 (magnetometer using superconducting rotating body), U.S. Pat. No. 4,349,781 (superconducting gradiometer-magnetometer array for magnetotelluric logging), U.S. Pat. Nos. 4,672,359, 4,804,915 (Squid magnetometer), U.S. Pat. No. 4,906,930 (magnetometer using a Josephson device and superconducting phototransistor), U.S. Pat. No. 4,923,850 (superconducting DC SQUID magnetometer working in liquid nitrogen), U.S. Pat. No. 5,008,622 (superconductive imaging surface magnetometer), U.S. Pat. No. 5,065,582 (Dewar vessel for a superconducting magnetometer device), U.S. Pat. No. 5,155,434 (superconducting quantum interference magnetometer having a plurality of gated channels), U.S. Pat. No. 5,184,072 (apparatus for measuring weak static magnetic field using sueprconduction strips and a SQUID magnetometer), U.S. Pat. No. 5,294,884 (high sensitive and high response magnetometer by the use of low inductance superconducting loop including a negative inductance generating means), U.S. Pat. No. 5,467,015 (superconducting magnetometer having increased bias current tolerance), U.S. Pat. No. 5,506,200 (compact superconducting magnetometer having no vacuum insulation), U.S. Pat. No. 6,225,800, and the like. The entire disclosure of each of these United States patents is hereby incorporated by reference into this specification.

In general, one may measure the d.c. slope at such point 1632 and the AC minor loop slope at such point 1632 by a process and apparatus depicted in FIG. 31. Referring to FIG. 31, a superconducting coil 1710 is disposed in the measurement set up 1700. The superconducting coil preferably has a length 1712 of about 1.5 feet, a diameter 1714 of about 1 foot, and a d.c. field strength of from about 0.5 to about 10 Tesla. Such a coil is well known in the art.

Referring again to FIG. 31, a d.c. pickup coil 1716 is disposed in set up 1700 such that a specimen 1718 is disposed between the pickup coil 1716 and the superconducting coil 1710. The specimen generally is one centimeter by one centimeter, with a width of one millimeter.

An a.c. field coil 1720 is disposed orthogonally to line 1722 defined by the d.c. pickup coil 1716 and the superconducting coil 1710. Such a.c. field coil preferably generates an electromagnetic field with a frequency of either 64 megahertz or 128 megahertz, depending upon the strength of the d.c. field produced by coil 1710.

The alternating current magnetic field produced by coil 1720 preferably has a magnitude of from about 10 to about 60 microTesla. In one embodiment, the magnitude of this a.c. magnetic field is from about 15 to about 25 microTesla.

Referring again to FIG. 31, disposed opposite to the a.c. coil 1710 is an alternating current pickup coil 1724 that also is orthogonal to line 1722. In one embodiment, the line 1726 between coil 1720 and coil 1724 is orthogonal to line 1722. As will be apparent, the set up 1700 is but one of many different way of utilizing the components in FIG. 31.

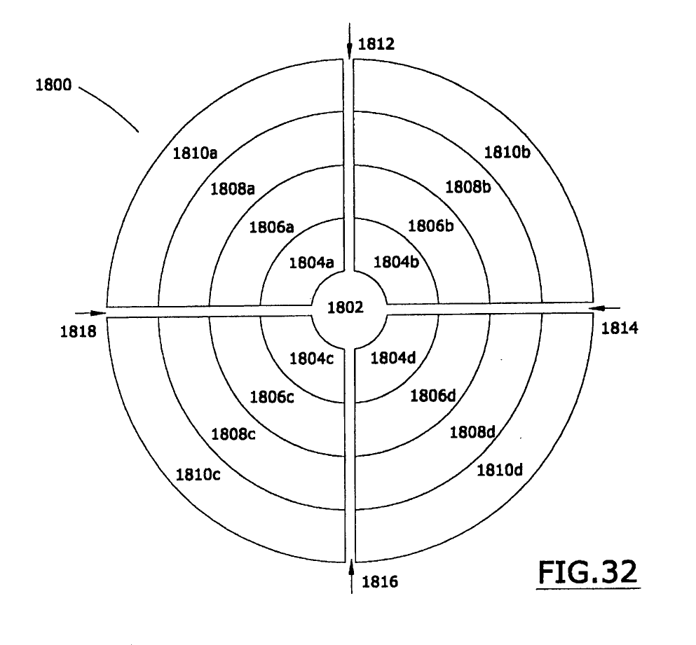

In one embodiment, illustrated in FIG. 32, a coated substrate assembly 1800 is depicted that is comprised of a metallic substrate 1802 and, disposed thereon, discontinuous coatings 1804a, 1804b, 1804c, 1804d, 1806a, 1806b, 1806c, 1806d, 1808a, 1808b, 1808c, 1808d, and 1810a, 1810b, 1810c, and 1810d.

The 1804a/b/c/d coatings are coatings of nanomagnetic material, such as the material in coating 1312 (see FIG. 27). The 1806a/b/c/d coatings are coatings of dielectric material, such as, e.g., material 1316 (see FIG. 27). The 1808a/b/c/d coatings are coatings of conductive material. The 1810a/b/c/d coatings are coatings that may comprise nanomagnetic material (as is present in coatings 1804) and/or may be hydrophilic and/or hydrophobic; as will be apparent, the stacking sequence 1804/1806/1804 may be repeated and/or altered to create many different combinations of equivalent inductors and/or equivalent capacitors and/or equivalent resistors connected in series and/or parallel and/or in series/parallel. This may be done to achieve the desired effects depicted in the equivalent circuit of FIG. 28.

As will be apparent, the various segments of coatings 1804, 1806, 1808 and 1810 are discontinuous. They may be connected, in part or in whole, by either insulating vias 1812 and 1814, and/or in part or in whole by conductive vias 1816 and 1818. In one embodiment, not shown, dielectric vias are also utilized to create many different combinations of equivalent inductors and/or equivalent capacitors and/or equivalent resistors connected in series and/or parallel and/or in series/parallel. This may be done to achieve the desired effects depicted in the equivalent circuit of FIG. 28.

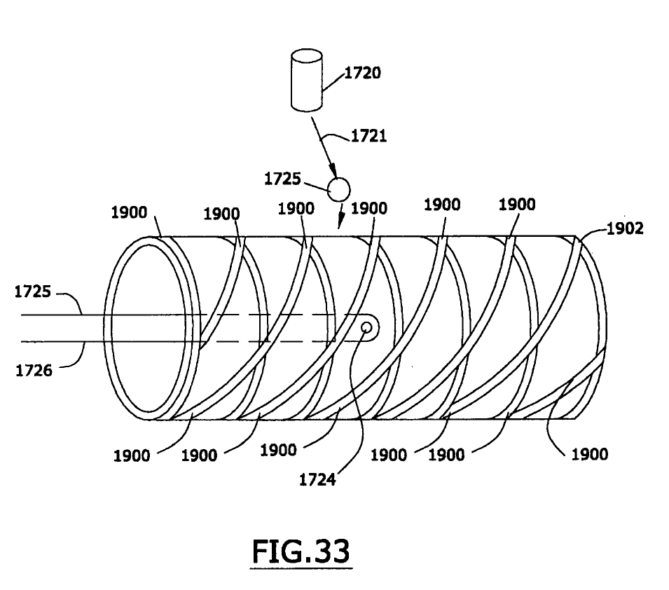

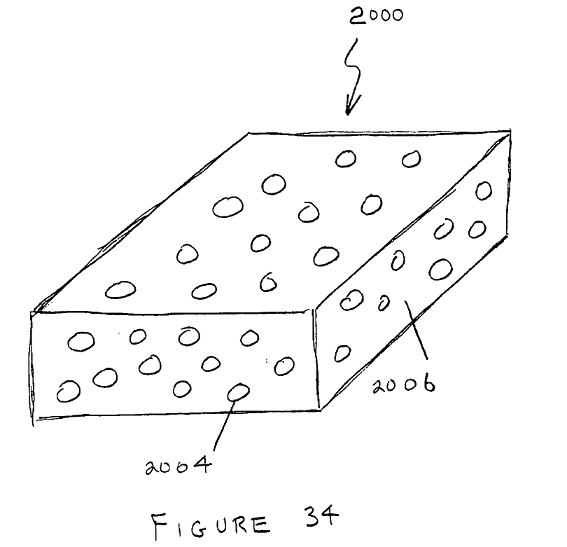

FIG. 33 illustrates the effect of a preferred coating 1900 on a stent 1902 that, in the embodiment depicted, is preferably a metallic stent.

One may use any of the metallic stents known to those skilled in the art. Thus, and referring to Patrick W. Serruys et al.'s “Handbook of Coronary Stents,” (Martin Dunitz Ltd, 2002), the stent may be a stainless steel “ARTHOS” stent with our without an inert surface (see pages 3-4), a 316L stainless steel “ANTARES STARFLEX” stent with a polished surface (see page 11), a 316 LVM stainless steel “SIRIUS” stent (see page 52), a 316L medical grade steel “GENIC” stent (see page 102), a Nitinol “BIFLEX” stent (see page 140), a niobium alloy “LUNAR” stent (see page 143), a stainless steel plated with gold “NIROYAL” stent (see page 219), a 316L stainless steel coated with hypothrombogenic alpha-SiCH:H “RITHRON” stent (see page 253), a 316L stainless steel with diamond-like carbon coating “PHYTIS” stent (see page 328), and the like.

This preferred coating, for reasons discussed elsewhere in this specification, allows the penetration of alternating current fields into the interior of the stent 1902.

Referring to FIG. 33, and in the preferred embodiment depicted therein, an alternating current field coil 1720 (see FIG. 31) is disposed outside of the stent 1902. In the embodiment depicted in FIG. 33, such a.c. field coil 1720 preferably generates an electromagnetic field with a frequency of either 64 megahertz or 128 megahertz, depending upon the strength of the d.c. field produced by coil 1710. Additionally, the alternating current magnetic field produced by coil 1720 preferably has a magnitude of from about 10 to about 60 microTesla. In one embodiment, the magnitude of this a.c. magnetic field is from about 15 to about 25 microTesla.

Referring again to FIG. 33, another source (not shown) generates a direct current field 1904 that either is at 1.5 Tesla or 3.0 Tesla and corresponds to a frequency of either 64 megahertz or 128 megahertz.

Disposed within the stent 1902 is A.C. pickup coil 1724 that comprise pickup coil leads 1725.

With the arrangement depicted in FIG. 33, one can determine the extent to which, if any, the alternating current electromagnetic field 1721 produced by a.c. field generator 17120 penetrates to the inside of stent 1902 and is detected by ac. pickup coil 1724. The difference between the a.c. field generated by coil 1720 and detected by coil 1724 divided by filed detected by coil 1724 is the “blockage;” and the blockage factor, in percent, is the blockage divided by the the a.c. filed generated by coil 1720 times 100.

With the arrangement depicted in FIG. 33, one may determine the blockage factor for an uncoated stent 1902. Thereafter, one can coat the identical stent and determine the blockage factor for this coated stent 1902. When stent 1902 is coated, its blockage factor will always be less than the blockage factor of the uncoated stent.

The ratio of the blockage factor of the uncoated stent/the blockage factor of the coated stent is referred to in this specification as the “transmission factor” of the coating. The preferred coatings of this invention, such as, e.g., coating 1312, have a transmission factor of at least about 1.5 and, preferably, at least about 2. In one preferred embodiment, the transmission factor of the nanomagnetic coatings of this invention are at least 3.

Additional Non-Magnetic Embodiments of the Invention

FIG. 34 is a schematic illustration, not drawn to scale, of a section of a material 2000 that is an embodiment of a marker material of the present invention. When disposed upon medical devices used during interventional medical procedures such as X-ray fluoroscopy and Magnetic Resonance Imaging, material 2000 will render such medical devices visible. In this embodiment material 2000 is comprised of particulate material 2004 disposed in a matrix material 2006.

Particulate material 2004 may be depicted by the formula AAₓC_{y} where A is an atom of an element having an atomic weight from about 30 to 300, preferably from about 50 to 260. Typically atoms A may be any of the elements Chromium, Manganese, Iron, Cobalt, Nickel, Copper, Zinc, Gallium, Germanium, Selenium, Strontium, Yttrium, Zirconium, Molybdenum, Neodymium, Samarium, Gadolinium, and the like. C may be an atom of any other element capable of combining with A and having an atomic weight less than 30, preferably less than 20. Subscripts x and y are binary numbers, 0 or 1, serving to indicate the presence or absence of the subscripted atom in particulate material 2004. Thus if x=0 and y=0, particulate material 2004 is comprised of only one type of A atoms. If x=1 and y=0, particulate material 2004 is comprised of only two types of A atoms. If x=0 and y=1, particulate material 2004 is comprised of one type of A atoms and C atoms. If x=1 and y=1, particulated material 2004 is comprised of two types of A atoms and C atoms.

Matrix material 2006 may be depicted by the formula B_{z}C_{w}, where B is an atom, different from C, but also of any element having an atomic weight less than 30, preferably less than 20. Subscripts z and w are again binary numbers, 0 or 1, serving to indicate the presence or absence of the subscripted atom in matrix material 2006. Typically atoms B and C may be any of the elements Boron, Carbon, Nitrogen, Oxygen, Magnesium, Aluminum, Silicon, and the like, as well as combinations thereof. In one embodiment the atomic weight of A is greater than the atomic weight of B or C. Preferably the atomic weight of A is at least two times tha atomic weight of B or C.

In one embodiment the particle size of particulate material 2004 is less than 100 nanometers, preferably less than 60 nanometers. In one embodiment the electrical conductivity of particulate material 2004 will generally be at least three times as great as the electrical conductivity of matrix material 2006. In one embodiment the dielectric constant of particulate material 2004 will be less than the dielectric constant of matrix material 2006, generally being from about 0.3 to 0.5 of the dielectric constant of matrix material 2006.

Particulate material 2004 may be a magnetic material such as, for example, one of the transition series metals that include chromium, manganese, iron, cobalt, nickel or combinations or alloys thereof. Embodiments of the invention in which particulate material 2004 is a magnetic material are disclosed in detail above.

Particulate material 2004 may also be non-magnetic material. In a non-magnetic embodiment, particulate material 2004 will have a low surface resistivity of about 1000×10⁻⁸ Ω-m or less. In one embodiment the surface resistivity of a non-magnetic particulate material 2004 is 10×10⁻⁸ Ω-m. Without wishing to be bound to any particular theory, applicants believe that the low surface resistivity of particulate material 2004, in a non-magnetic embodiment, will result in induced surface currents when exposed to the RF electromagnetic radiation during magnetic resonance imaging, thereby resulting in high visibility in that imaging modality.

Without wishing to be bound to any particular theory, applicants believe that the unique ability of material 2000 to function well as a visibility marker in both X-ray fluoroscopy and MR imaging modalities is due to particulate material 2004 being near the imaging resolution limit of both modalities. Thus, e.g., under MRI the visibility of the medical device upon which material 2000 is disposed is enhanced by particulate material 2004 creating a local inhomogeniety, but not a global inhomogeniety. Under X-ray imaging the particulate material 2004 particles are good X-ray absorbers, due to the relationship of their size to the wavelength of the X-radiation. In one embodiment the absorption of X-rays by particulate material 2004 is at least twice as great as that of matrix material 2006.

In one embodiment marker material 2000 is disposed upon a medical device in the form of a coating with a thickness greater that 400 nanometers, preferably greater than 600 nanometers. In such embodiment marker material 2000 may be disposed over essentially the whole medical device, or alternatively marker material 2000 may be disposed only over partial sections if the medical device. For example, if the medical device is a catheter for insertion into a patient's artery, only the distal end of the catheter might be coated with marking material 2000 if it is only necessary for that section of the catheter to be visible during the interventional procedure. In another embodiment marker material 2000 is coated onto the medical device in different patterns at different sections of the medical device.

While preferred embodiments of this invention have been shown and described, modifications thereof can be made by one skilled in the art without departing from the spirit or teaching of this invention. The embodiments described herein are exemplary only and are not limiting. Many variations of the method are possible and were within the scope of the invention.

1. A medical device assembly that is insertable into the body of a patient, said medical device assembly comprising: a surface and a marking material disposed on at least a portion of said surface, wherein said marking material is comprised of a particulate material disposed in a matrix material, wherein said particulate material is comprised of a plurality of first atoms and a plurality of second atoms, wherein said matrix material is comprised of a plurality of said second atoms and a plurality of third atoms, wherein said first atoms have an atomic weight at least twice the atomic weight of said second atoms and twice the atomic weight of said third atoms, and wherein the average particle size of said particulate material is less than 100 nanometers.

2. The medical device assembly recited in claim 1, wherein the atomic weight of said first atoms is in the range from about 30 to about 300.

3. The medical device assembly recited in claim 2, wherein the atomic weight of said first atoms is in the range from about 50 to about 260.

4. The medical device assembly recited in claim 3, wherein said first atoms are chosen from the group consisting of Chromium, Manganese, Iron, Cobalt, Nickel, Copper, Zinc, Gallium, Germanium, Selenium, Strontium, Yttrium, Zirconium, Molybdenum, Neodymium, Samarium, Gadolinium, and combinations thereof.

5. The medical device assembly recited in claim 3, wherein the atomic weight of said second atoms and the atomic weight of said third atoms is less than 30.

6. The medical device assembly recited in claim 5, wherein said second atoms and said third atoms are chosen from the group consisting of Boron, Carbon, Nitrogen, Oxygen, Magnesium, Aluminum, Silicon, and combinations thereof.

7. The medical device assembly recited in claim 5, wherein the atomic weight of said second atoms and the atomic weight of said third atoms is less than 20.

8. The medical device assembly recited in claim 5, wherein the electrical conductivity of said particulate material is at least three times the electrical conductivity of said matrix material.

9. The medical device assembly recited in claim 5, wherein the dielectric constant of said particulate material is less than the dielectric constant of said matrix material.

10. The medical device assembly recited in claim 9, wherein the dielectric constant of said particulate material is about 0.3 to about 0.5 of the dielectric constant of said matrix material.

11. The medical device assembly recited in claim 5, wherein said particulate material is non-magnetic.

12. The medical device assembly recited in claim 11, wherein said particulate material has an electrical surface resistivity of 1000×10⁻⁸ Ω-m or less.

13. The medical device assembly recited in claim 12, wherein said particulate material has an average particle size of less than 60 nanometers.

14. The medical device assembly recited in claim 12, wherein said particulate material has an electrical surface resistivity of 10×10⁻⁸ Ω-m or less.

15. The medical device assembly recited in claim 14, wherein said particulate material has an average particle size of less than 60 nanometers.

16. The medical device assembly recited in claim 5, wherein said particulate material is a magnetic material.

17. The medical device assembly recited in claim 16, wherein said first atoms are chosen from the group consisting of Cr, Mn, Fe, Co, Ni, and combinations thereof.

18. The medical device assembly recited in claim 5, wherein said particulate material is comprised of nanomagnetic particles.

19. The medical device assembly recited in claim 18, wherein said nanomagnetic particles have a direct current permeability at a static magnetic field of 1.5 Tesla of from about 1.1 to about 2.0.

20. The medical device assembly recited in claim 18, wherein the average particle size of said nanomagnetic particles is from about 3 to about 10 nanometers.

21. The medical device assembly recited in claim 18, wherein said nanomagnetic particles have a coherence length of less than 100 nanometers.

22. The medical device assembly as recited in claim 18, wherein said nanomagnetic particles have an average particle size of less than about 20 nanometers and a phase transition temperature of less than about 200 degrees Celsius.

23. The medical device assembly as recited in claim 18, wherein the average particle size of said nanomagnetic particles is less than about 15 nanometers.

24. The medical device assembly as recited in claim 18, wherein said nanomagnetic particles have a squareness of from about 0.05 to about 1.0.

25. The medical device assembly as recited in claim 18, wherein said nanomagnetic particles have a squareness of from about 0.1 to about 0.9.

26. The medical device assembly as recited in claim 18, wherein said nanomagnetic particles have a squareness of from about 0.2 to about 0.8.

27. The medical device assembly as recited in claim 18, wherein said nanomagnetic particles have an average particle size of less than about 3 nanometers.

28. The medical device assembly as recited in claim 18, wherein said nanomagnetic particles have a phase transition temperature of less than about 46 degrees Celsius.

29. The medical device assembly as recited in claim 18, wherein said nanomagnetic particles have a phase transition temperature of less than about 50 degrees Celsius.

30. The medical device assembly as recited in claim 18, wherein said nanomagnetic particles have a coercive force of from about 0.01 to about 5,000 Oersteds.

31. The medical device assembly as recited in claim 18, wherein said first atoms are comprised of iron and said second atoms are comprised of aluminum.

32. The medical device assembly as recited in claim 31, wherein said nanomagnetic particles are comprised of less than 50 weight percent of iron, by total weight of iron and aluminum.

33. The medical device assembly as recited in claim 31, wherein said nanomagnetic particles are comprised of from about 5 to about 40 weight percent of iron, by total weight of iron and aluminum.

34. The medical device assembly as recited in claim 31, wherein said nanomagnetic particles are comprised of from about 5 to about 30 weight percent of iron, by total weight of iron and aluminum.

35. The medical device assembly as recited in claim 31, wherein said nanomagnetic particles are comprised of from about 5 to about 20 weight percent of iron, by total weight of Iron and aluminum.

36. The medical device assembly as recited in claim 5, wherein said medical device is a catheter.

37. The medical device assembly as recited in claim 5, wherein said medical device is a stent.

38. A medical device assembly that is insertable into the body of a patient, said medical device assembly comprising: a surface and a marking material disposed in the form of a film on at least a portion of said surface, wherein said film has a thickness of from about 400 to about 2000 nanometers, wherein said marking material is comprised of a particulate material disposed in a matrix material, wherein said particulate material is comprised of a plurality of first atoms and a plurality of second atoms, wherein said matrix material is comprised of a plurality of said second atoms and a plurality of third atoms, wherein said first atoms have an atomic weight at least twice the atomic weight of said second atoms and twice the atomic weight of said third atoms, and wherein the average particle size of said particulate material is less than 100 nanometers.

39. The medical device assembly as recited in claim 38, wherein said film has a thickness of from about 600 to about 1200 nanometers.

40. The medical device assembly recited in claim 38, wherein the atomic weight of said first atoms is in the range from about 30 to about 300.

41. The medical device assembly recited in claim 40, wherein the atomic weight of said first atoms is in the range from about 50 to about 260.

42. The medical device assembly recited in claim 41, wherein said first atoms are chosen from the group consisting of Chromium, Manganese, Iron, Cobalt, Nickel, Copper, Zinc, Gallium, Germanium, Selenium, Strontium, Yttrium, Zirconium, Molybdenum, Neodyium, Samarium, Gadolinium, and combinations thereof.

43. The medical device assembly recited in claim 41, wherein the atomic weight of said second atoms and the atomic weight of said third atoms is less than 30.

44. The medical device assembly recited in claim 43, wherein said second atoms and said third atoms are chosen from the group consisting of Boron, Carbon, Nitrogen, Oxygen, Magnesium, Aluminum, Silicon, and combinations thereof.

45. The medical device assembly recited in claim 43, wherein the atomic weight of said second atoms and the atomic weight of said third atoms is less than 20.

46. The medical device assembly recited in claim 43, wherein the electrical conductivity of said particulate material is at least three times the electrical conductivity of said matrix material.

47. The medical device assembly recited in claim 43, wherein the dielectric constant of said particulate material is less than the dielectric constant of said matrix material.

48. The medical device assembly recited in claim 47, wherein the dielectric constant of said particulate material is about 0.3 to about 0.5 of the dielectric constant of said matrix material.

49. The medical device assembly recited in claim 43, wherein said particulate material is non-magnetic.

50. The medical device assembly recited in claim 49, wherein said particulate material has an electrical surface resistivity of 1000×10⁻⁸ Ω-m or less.

51. The medical device assembly recited in claim 50, wherein said particulate material has an average particle size of less than 60 nanometers.

52. The medical device assembly recited in claim 50, wherein said particulate material has an electrical surface resistivity of 10×10⁻⁸ Ω-m or less.

53. The medical device assembly recited in claim 52, wherein said particulate material has an average particle size of less than 60 nanometers.

54. The medical device assembly recited in claim 43, wherein said particulate material is a magnetic material.

55. The medical device assembly recited in claim 54, wherein said first atoms are chosen from the group consisting of Cr, Mn, Fe, Co, Ni, and combinations thereof.

56. The medical device assembly recited in claim 43, wherein said particulate material is comprised of nanomagnetic particles.

57. The medical device assembly recited in claim 56, wherein said nanomagnetic particles have a direct current permeability at a static magnetic field of 1.5 Tesla of from about 1.1 to about 2.0.

58. The medical device assembly recited in claim 56, wherein the average particle size of said nanomagnetic particles is from about 3 to about 10 nanometers.

59. The medical device assembly recited in claim 56, wherein said nanomagnetic particles have a coherence length of less than 100 nanometers.

60. The medical device assembly as recited in claim 56, wherein said nanomagnetic particles have an average particle size of less than about 20 nanometers and a phase transition temperature of less than about 200 degrees Celsius.

61. The medical device assembly as recited in claim 56, wherein the average particle size of said nanomagnetic particles is less than about 15 nanometers.

62. The medical device assembly as recited in claim 56, wherein said nanomagnetic particles have a squareness of from about 0.05 to about 1.0.

63. The medical device assembly as recited in claim 56, wherein said nanomagnetic particles have a squareness of from about 0.1 to about 0.9.

64. The medical device assembly as recited in claim 56, wherein said nanomagnetic particles have a squareness of from about 0.2 to about 0.8.

65. The medical device assembly as recited in claim 56, wherein said nanomagnetic particles have an average particle size of less than about 3 nanometers.

66. The medical device assembly as recited in claim 56, wherein said nanomagnetic particles have a phase transition temperature of less than about 46 degrees Celsius.

67. The medical device assembly as recited in claim 56, wherein said nanomagnetic particles have a phase transition temperature of less than about 60 degrees Celsius.

68. The medical device assembly as recited in claim 56, wherein said nanomagnetic particles have a coercive force of from about 0.01 to about 5,000 Oersteds.

69. The medical device assembly as recited in claim 56, wherein said first atoms are comprised of iron and said second atoms are comprised of aluminum.

70. The medical device assembly as recited in claim 69, wherein said nanomagnetic particles are comprised of less than 50 weight percent of iron, by total weight of iron and aluminum.

71. The medical device assembly as recited in claim 69, wherein said nanomagnetic particles are comprised of from about 5 to about 40 weight percent of iron, by total weight of iron and aluminum.

72. The medical device assembly as recited in claim 69, wherein said nanomagnetic particles are comprised of from about 5 to about 30 weight percent of iron, by total weight of iron and aluminum.

73. The medical device assembly as recited in claim 69, wherein said nanomagnetic particles are comprised of from about 5 to about 20 weight percent of iron, by total weight of iron and aluminum.

74. The medical device assembly as recited in claim 43, wherein said medical device is a catheter.

75. The medical device assembly as recited in claim 43, wherein said medical device is a stent.
